SEQUENCE LISTING (21 sequences)

SEQ ID NO: 1 - protein, 321 aa, Escherichia coli
  source 1..321: note = EPEC EspB Accession number: WP_001091991.1
MNTIDNNNAAIAVNSVLSSTTDSTSSTTTSTSSISSSLLTDGRVDISKLLLEVQKLLREMVTTLQDYLQKQLAQSYDIQKAVFESQNKAIDEKKAGATAALIGGAISSVLGILGSFAAINSATKGASDVAQQAASTSAKSIGTVSEASTKALAKASEGIADAADDAAGAMQQTIATAAKAASRTSGITDDVATSAQKASQVAEEAADAAQELAQKAGLLSRFTAAAGRISGSTPFIVVTSLAEGTKTLPTTISESVKSNHDINEQRAKSVENLQASNLDTYKQDVRRAQDDISSRLRDMTTTARDLTDLINRMGQAARLAG

SEQ ID NO: 2 - DNA, 945 bp, Escherichia coli
  source 1..945: mol_type = genomic DNA; note = EspB Accession number: AAB69980.1
atgaatactattgattatactaatcaagtaatgacggttaattctgtttcggagaatactaccggctctaatgcaattaccgcatctgctattaattcatctttgcttaccgatggtaaggtcgatgtttctaaactgatgctggaaattcaaaaactcctgggcaagatggtgcgtatattgcaggattaccaacagcaacagttgtcgcagagctatcagatccaactggccgtttttgagagccagaataaagccattgatgaaaaaaaggccgctgcaacagccgctctggttggtggggctatttcatcagtattggggatcttaggctcttttgcagcaattaacagtgctacgaaaggcgcgagtgatattgctcaaaaaaccgcctctacatcttctaaggctattgatgcggcttctgatactgcgactaaaacgttgactaaggcaacggaaagcgttgctgatgctgttgaagatgcatccagcgtgatgcagcaagcgatgactacagcaacgagagcggccagccgtacatccgacgttgctgatgacattgccgattctgctcagagagcttctcagctggctgaaaacgctgcagatgccgctcagaaggcaagtcgggcaagccgctttatggctgcagtagataagattactggctctacaccatttattgccgttaccagtcttgccgaaggcacgaagacattgccaacaacggtatctgaatcagtcaaatctaaccatgagattagcgaacagcgttataagtctgtggagaacttccagcagggtaatttggatctgtataagcaagaagttcgcagagcgcaggatgatatcgctagccgtctgcgtgatatgacaacagccgctcgcgatctcactgatcttcagaatcgtatgggtcaatcggttcgcttagctgggtaa

SEQ ID NO: 3 - protein, 380 aa, Escherichia coli
  source 1..380: note = EspD Accession number: WP_000935767.1
MLNVNSDIQSMRSGASAATATSGINQPEVTSALDLQLVKSTAPSASWTESTALPTPPAGHSLVTPSAAEDVLSKLFGGISGEVRSRTEGTEPQRSTQNASSGYPYLSQVNNVDPQAMMMMVTLLSLDASAQKVASMKNSNEIYTDGQNKALDNKTLEFKKQLEEQQKAEEKAQKSKIVGQVFGWLGVAVTAIAAIFNPALWAVVAVSATAMALQTAVDVMGDKAPQALKTAAQVFGGVSIAASILTAGIGGVSSLMSKVGDVANKVGSNIVKTVTKLADVFVENVVSKVAATANGFTTSARSIGTTVLNNDSAQYSILAQLSAYAVQNLTRQSENLGESAKVELDKAAAELRNQASYLQNASQLISDSARVNSRIVSGRV

SEQ ID NO: 4 - DNA, 1000 bp, Escherichia coli
  source 1..1000: mol_type = genomic DNA; note = EspD Accession number: CAI43861.1
atgctcaatgtaaatagcgatatccagtcgatgaggtctggggccagcgctgctacggctacttcaggtattaatcagcctgaagtgacttccgcgctggatttacaactggttaaatccacggccccatcagcaagttggacagaaagtaccgctttaccaacgccaccggcaggccactcattagtgacgccctctgctgctgaggatgtcctgagtaaactgtttggtggtattagtggtgaagttaggagtcgaacagagggaaccgaaccacagcgcagcactcagaatgcaagctctggctatccttatctttctcaggtgaataacgttgaccctcaggccatgatgatgatggtcaccctgttatccctggatgcttcagcgcagaaagttgcgagtatgaaaaactccaacgagatctacacggatgggcaaaataaggcgctggataataaaacgctggagtttaaaaagcagctcgaagaacaacagaaagccgaagagaaagcacaaaaaagtaaaattgttggtcaggtctttggttggttgggcgtcgcggtaactgctattgcagctatttttaacccggcactctgggctgttgttgccgttagcgcaacagcaatggcactgcaaactgcagttgatgtaatgggcgataaggccccgcaggcgttaaagacagcagcgcaagtctttggcggagtatctattgctgcaagtattctgacagccggcattggcggggtgtcttcacttatgtctaaagttggcgatgtcgctaacaaagttggttcaaacatcgtgaaaacagttactaaactggccgatgtatttgttgagaacgttgtttcaaaagttgcggcaacggcaaatggatttacgacctctgcacgttctattgggacaactgtgctgaataatgattctgcgcaatacagcatattggctcagttatctgcttacgcggtgcaaaacttaactcgacagagtg

SEQ ID NO: 5 - protein, 11 aa, synthetic construct
  source 1..11: note = EPITOPE OF B7
KASQVAEEAAD

SEQ ID NO: 6 - protein, 8 aa, synthetic construct
  source 1..8: note = B7-VH CDRH1
GFTFSHYA

SEQ ID NO: 7 - protein, 8 aa, synthetic construct
  source 1..8: note = B7-VH CDRH2
INSNGDST

SEQ ID NO: 8 - protein, 12 aa, synthetic construct
  source 1..12: note = B7- VL CDRL1
ARDRRAGYFDYW

SEQ ID NO: 9 - protein, 8 aa, synthetic construct
  source 1..8: note = B7- VL CDRL1
RDNIGKNY

SEQ ID NO: 10 - protein, 192 aa, Escherichia coli
  source 1..192: note = UniProtKB - Q47184 (Q47184_ECOLX) >tr|Q47184|Q47184_ECOLX EspA OS=Escherichia coli OX=562; GN = espA PE=1 SV=1
MDTSTTASVASANASTSTSMAYDLGSMSKDDVIDLFNKLGVFQAAILMFAYMYQAQSDLSIAKFADMNEASKESTTAQKMANLVDAKIADVQSSSDKNAKAQLPDEVISYINDPRNDITISGIDNINAQLGAGDLQTVKAAISAKANNLTTTVNNSQLEIQQMSNTLNLLTSARSDMQSLQYRTISGISLGK

SEQ ID NO: 11 - protein, 9 aa, synthetic construct
  source 1..9: note = B7- VL CDRL3
SAWDTSLNA

SEQ ID NO: 12 - DNA, 354 bp, synthetic construct
  source 1..354: mol_type = other DNA; note = B7-VH Full Sequence
gaggtgcagctgttggagtctgggggaggcttggtacagcctggggggtccctgagactctcctgtgcagcctctggattcacctttagccactatgccatgaactgggtccgccaggctccagggaaggggctggagtgggtttcagccattaatagtaatggtgatagcacctactatccagacactgtgaaggaccgattcaccatctccagagacaattccaagaacacgctgtatctgcaaatgaacagcctgagagccgaggacacggccgtgtattactgtgcgagagatcggcgagcagggtactttgactactggggccagggcaccctggtcacagtctcctca

SEQ ID NO: 13 - protein, 118 aa, synthetic construct
  source 1..118: note = B7-VH Full Sequence
EVQLLESGGGLVQPGGSLRLSCAASGFTFSHYAMNWVRQAPGKGLEWVSAINSNGDSTYYPDTVKDRFTISRDNSKNTLYLQMNSLRAEDTAVYYCARDRRAGYFDYWGQGTLVTVSS

SEQ ID NO: 14 - DNA, 330 bp, synthetic construct
  source 1..330: mol_type = other DNA; note = B7- VL (lambda) Full Sequence
gatatcgtgctgactcagccaccctcagcgtctgggacccccgggcagagggtcaccatctcctgctctggaagcagggacaacatcgggaagaattatgtgtcctggtaccagcagcttccaggaacagcccccaaactcctcatttataggaataatcagcggccctcaggggtccctgaccggttctctggctccaagtctggcacctcagcctccctggccatcagtgggctgcggtccgaggatgaggctgattattactgctcagcgtgggacaccagcctcaatgcttatgtcttcgggcctggcacccagctcaccgtcctc

SEQ ID NO: 15 - protein, 110 aa, synthetic construct
  source 1..110: note = B7- VL (lambda) Full Sequence
DIVLTQPPSASGTPGQRVTISCSGSRDNIGKNYVSWYQQLPGTAPKLLIYRNNQRPSGVPDRFSGSKSGTSASLAISGLRSEDEADYYCSAWDTSLNAYVFGPGTQLTVL

SEQ ID NO: 16 - protein, 25 aa, synthetic construct
  source 1..25: note = B7-VH FR1
EVQLLESGGGLVQPGGSLRLSCAAS

SEQ ID NO: 17 - protein, 17 aa, synthetic construct
  source 1..17: note = B7-VH FR2
MNWVRQAPGKGLEWVSA

SEQ ID NO: 18 - protein, 38 aa, synthetic construct
  source 1..38: note = B7-VH FR3
YYPDTVKDRFTISRDNSKNTLYLQMNSLRAEDTAVYYC

SEQ ID NO: 19 - protein, 25 aa, synthetic construct
  source 1..25: note = B7- VL FR1
DIVLTQPPSASGTPGQRVTISCSGS

SEQ ID NO: 20 - protein, 17 aa, synthetic construct
  source 1..17: note = B7- VL FR2
VSWYQQLPGTAPKLLIY

SEQ ID NO: 21 - protein, 36 aa, synthetic construct
  source 1..36: note = B7- VL FR3
QRPSGVPDRFSGSKSGTSASLAISGLRSEDEADYYC

(19) United States
(12) Patent Application Publication
(10) Pub. No.: US 2025/0321227 A1
(43) Pub. Date: Oct. 16, 2025

(54) DUAL-MODE ELECTROCHEMICAL POINT-OF-CARE DETECTION, QUANTIFICATION AND PROFILING OF PATHOGENS

(71) Applicants: THE STATE OF ISRAEL, MINISTRY OF AGRICULTURE & RURAL DEVELOPMENT, AGRICULTURAL RESEARCH ORGANIZATION, Rishon Lezion (IL); B.G. NEGEV TECHNOLOGIES AND APPLICATIONS LTD., AT BEN-GURION UNIVERSITY, Beer Sheva (IL)

(72) Inventors: Neta SAL-MAN, Beer Shava (IL); Sefi VERNICK, Rishon Lezion (IL); Yariv WINE, Tel Aviv (IL)

(73) Assignees: THE STATE OF ISRAEL, MINISTRY OF AGRICULTURE & RURAL DEVELOPMENT, AGRICULTURAL RESEARCH ORGANIZATION, Rishon Lezion (IL); B.G. NEGEV TECHNOLOGIES AND APPLICATIONS LTD., AT BEN-GURION UNIVERSITY, Beer Sheva (IL)

(21) Appl. No.: 18/866,658

(22) PCT Filed: May 18, 2023
(86) PCT No.: PCT/IL2023/050516

§ 371 Date: Nov. 18, 2024

Related U.S. Application Data
(60) Provisional application No. 63/343,272, filed on May 18, 2022.

(51) Int. Cl. G01N 33/543; C12Q 1/00; G01N 27/02; G01N 27/327; G01N 27/48; G01N 33/569

(52) U.S. Cl. CPC G01N 33/5438; C12Q 1/005; G01N 27/026; G01N 27/3276; G01N 27/48; G01N 33/56916; G01N 2333/245; G01N 2469/10

(57) ABSTRACT

The present invention relates to diagnostic platform. More specifically, the invention relates to a dual platform composed of a detection and quantification unit and a characterization unit, systems, kits, methods and uses thereof in detection, quantification and characterization of pathogens, the system comprising monoclonal-antibody-based biosensor chips, for detection and/or quantification of pathogens in a sample, and substrate-based biosensor chips for enzymatically profiling the pathogen in the sample.

### FIELD OF THE INVENTION

The present invention generally relates to diagnostic platform. More specifically, the present disclosure relates to a dual platform composed of a detection and quantification unit and a characterization unit. The present disclosure further provides systems, kits, methods and uses of the disclosed diagnostic platform in detection, quantification and characterization of pathogens.

### BACKGROUND ART

References considered to be relevant as background to the presently disclosed subject matter are listed below:
1. Croxen, M. A.; Law, R. J.; Scholz, R.; Keeney, K. M.; Wlodarska, M.; Finlay, B. B., Recent advances in understanding enteric pathogenic Escherichia coli. Clin Microbiol Rev 2013, 26 (4), 822-80.
2. Guttikonda, S.; Tang, X. L.; Yang, B. M.; Armstrong, G. D.; Suresh, M. R., Monospecific and bispecific antibodies against E. coli O157 for diagnostics. J Immunol Methods 2007, 327 (1-2), 1-9.
3. Jampasa, S.; Lae-Ngee, P.; Patarakul, K.; Ngamrojanavanich, N.; Chailapakul, O.; Rodthongkum, N., Electrochemical immunosensor based on gold-labeled monoclonal anti-LipL32 for leptospirosis diagnosis. Biosens Bioelectron 2019, 142, 111539.
4. Kaper, J. B.; Nataro, J. P.; Mobley, H. L., Pathogenic Escherichia coli. Nature reviews. Microbiology 2004, 2 (2), 123-40.
5. Cornelis, G. R., The type III secretion injectisome. Nature reviews. Microbiology 2006, 4 (11), 811-25.
6. Hillman Y., et al., Monoclonal Antibody-Based Biosensor for Point-of-Care Detection of Type III Secretion System Expressing Pathogens. Anal. Chem. 2021, 93, 2, 928-935.
7. Randviir, E. P. and Banks, C. E. (2013) Electrochemical impedance spectroscopy: an overview of bioanalytical applications. Analytical methods 5(5), 1098-1115.
8. Lasia, A. (2014) Electrochemical Impedance Spectroscopy and its Applications, pp. 85-125, Springer
9. Ogungbile, A. O.; Ashur, I.; Icin, I.; Shapiro, O. H.; Vernick, S. 2021. Rapid detection and quantification of microcystins in surface water by an impedimetric immunosensor. Sensors and Actuators B: Chemical, 348, 130687
10. Ashur, I.; Alter, J.; Werbner, M.; Ogungbile, A.; Dessau, M.; Gal-Tanamy, M.; Vernick, S. 2022. Rapid electrochemical immunodetection of SARS-CoV-2 using a pseudo-typed vesicular stomatitis virus model. Talanta, 239, 123147
11. Gunasekaran, D.; Gerchman, Y.; Vernick, S. 2022. Electrochemical Detection of Waterborne Bacteria Using Bi-Functional Magnetic Nanoparticle Conjugates. Biosensors, 12 (1).

### BACKGROUND OF THE INVENTION

The emergence of multiple-drug resistant (MDR) bacterial strains stems largely from the extensive, and sometimes inappropriate, usage of antibiotics in the community and in agriculture, as this misuse has exerted a strong selective pressure on bacteria to develop resistance mechanisms against various antibiotics. In turn, the implications of the increasing numbers of MDR bacterial infections in the clinic, in the community, and in agriculture are constituting a growing global public health concern. MDR bacterial infections are harder to treat and are associated with higher medical costs than antibiotic-sensitive infections, and, perhaps more importantly, there is a significant risk that MDR mechanisms will be spread to other bacterial strains. A parallel public health concern is that the development and approval of new antibiotics has not kept pace with the rising rates of morbidity and mortality due to bacterial infections, giving rise to a predicted annual death rate of 10 million people by 2050 due to resistance to antimicrobials. The lack of progress in the development of antibiotics may be attributed not only to the limited discovery of suitable molecular targets, but also to the absence of significant investment of large pharmaceutical companies.

Pivotal to the efficiency of controlling antibiotic resistance is the ability to provide rapid and accurate surveillance and diagnosis, as is embodied in the WHO One Health concept for addressing the MDR crisis. In this regard, the major disadvantages of currently available laboratory-based diagnostics for the detection of bacterial infections are long processing times, low sensitivity and specificity, and/or the need for specialized equipment that is expensive and requires highly trained personnel. Among the laboratory-based methods currently in use for bacterial diagnosis, bacterial culturing is probably the most frequently used method, but it is relatively slow, and it is limited to bacteria that can be cultured in the laboratory. Other methods are based on immunoassays [including enzyme-linked immunosorbent assays (ELISA) and agglutination assays] that detect surface bacterial antigens and on genetic analyses that allow rapid identification of bacterial strains by employing a polymerase chain reaction (PCR). The latter methods are the most sensitive, but even they may yield false-positive results and they may overlook genetically mutated strains. A possible solution was thought to lie in rapid real-time PCR or mass-spectroscopy techniques, but these, too, require specialized equipment and reagents and trained personnel [1]. The above-described obstacles may culminate in misdiagnosed or belatedly diagnosed bacterial infections and the misuse of antibiotics, and hence, ultimately, in the exacerbation of the antibiotic resistance crisis.

Electrochemical (EC) biosensors facilitate direct electronic transduction of specific molecular binding into electrons, thus avoiding the use of optics, enabling low-form-factor devices, and delivering high signal levels. Additionally, EC biosensors have a virtually unlimited multiplexing potential since they are amenable to miniaturization and compatible with CMOS-integrating technologies, making them ideal for rapid on-site applications [7, 8].

A particularly promising means for providing such diagnosis lies in monoclonal antibodies (mAbs) targeted against pathogen-specific antigens. mAbs were previously demonstrated as diagnostic agents for the detection of harmful bacteria [2, 3]. In keeping with this line of thought, recent advances in the discovery, engineering, production, and clinical development of mAbs indicate their potential in the design of rapid and accurate diagnostics.

A major need for rapid diagnosis includes, but not limited to, strains of Gram-negative bacterial pathogens, such as Escherichia coli, and species of Salmonella, Shigella, Yersinia, and Pseudomonas, which cause serious diseases, ranging from lethal diarrhea to sepsis, leading to millions of deaths annually [1]. An essential component common to these bacterial pathogens is termed the type 3 secretion system (T3SS). The T3SS is a syringe-like protein complex, which is responsible for injecting virulence factors from the bacterial cytoplasm directly into the human host cell [4]. This T3SS complex is essential for bacterial virulence, as the injected proteins (effectors) manipulate key intracellular host pathways (e.g., cell cycle, immune response, cytoskeletal organization, metabolic processes and intracellular trafficking) that ultimately promote bacterial replication and transmission [5].

The present inventors previously described [6] the development of a T3SS-specific mAb and its use in a bioelectronic diagnostic device for the detection of enteropathogenic E. coli (EPEC). EPEC contains a T3SS, which is absent from the non-pathogenic strains of E. coli. The EPEC T3SS comprises more than 20 proteins, three of which, EspA, EspB, and EspD, are highly exposed to the extracellular environment. EspA forms a long filamentous structure that bridges the bacterial and host cells, and EspB and EspD together form a translocator pore complex that facilitates the passage of effectors across the host plasma membrane. Moreover, the inventors have recently developed EC biosensor based on a micro-fabricated chip equipped with a multi-electrode array, functionalized with antibodies that recognize pathogenic bacterial biomarkers within <30 min [6, 9]. The specific binding of biomarkers is translated into a measurable dose-response electronic signal. Furthermore, the inventors have now shown in the present disclosure the ability to detect enzymes that confer antibiotic resistance by measuring the current signal obtained by microelectrodes modified with antibiotics.

In addition to the detection, identification and quantification of pathogen/s in a sample, further characterization of the pathogen's abilities with regard to their enzymatic profile or their toxin production is highly valuable for clinical purposes.

Thus, sensitive systems that on one hand provides identification, quantification and capturing of the pathogens in a sample, using antibodies against T3SS components (such as antibodies directed at EspB), and on the other hand, substrate-based biosensors that provide enzymatic profiling of the pathogens of interest, specifically, antibiotic resistance profiling or toxin production, results in a powerful and rapid point-of-care combined diagnostic and therapeutic solution for monitoring bacterial infections in the community. There is, thus, an imperative need for more rapid, cost-effective, and sensitive assays that can identify infective agents at the point of care (POC), and characterize the pathogen, without the requirement for multistep processing.

### SUMMARY OF THE INVENTION

The inventors herein propose to develop a dual-mode biosensor device that integrates both abilities on a single microchip (FIG. 4). This chip enables rapid detection of both pathogenic biomarkers and enzymatic activity (with emphasis on these related to antibiotic resistance or virulence factors such as toxin production), providing a quantitative determination of drug-resistant pathogenic bacteria in a variety of samples including clinical, water, food, and environmental samples. The inventors effectively combine the inherent sensitivity of EC transduction with the selectivity and robustness of immuno-detection. By leveraging the advantages of microelectronics, a synergistic approach is offered with great potential for high-throughput diagnostic applications. The device of the present disclosure overcomes many of the limitations of established detection methods and promises to significantly expand the diagnostics capacity. Importantly, it greatly simplifies sample preparation, is capable of selectively detecting low concentrations of antibiotic-resistant pathogens and allows for a straightforward measurement of highly complex matrices.

A first aspect of the present disclosure relates to a system comprising:
(a) a pathogen identification and/or quantification unit comprising a first electrode arrangement comprising at least one first working electrode located within at least one first chamber; and
(b) a profiling unit comprising a second electrode arrangement comprising at least one second working electrode located within at least one second chamber. The second chamber is connected to the at least one first chamber, associated with the pathogen identification unit, to allow selective fluid transmission from the at least one first chamber to the at least one second chamber.

It should be noted that at least one first working electrode can be contacted directly or indirectly to at least one target binding site and/or moiety specific for binding one or more target pathogens. Still further, at least one second working electrode may be in the vicinity of, or may be connected directly or indirectly to at least one substrate molecule/s. It should be thus understood that in some embodiments, the at least one second electrode is attached, and/or connected directly, or indirectly via a connector or linker to the substrate. However, in some alternative or additional embodiments, at least some of the at least one second working electrodes are placed in the vicinity of a substrate, or in close proximity to at least one substrate, or in some embodiments may be placed together unconnected in at least one second chamber.

Still further, it should be understood that the suitable substrate used in the system of the present disclosure is characterized in that the interaction of such at least one substrate molecule/s with at least one catalytic macromolecule, catalyzes the production of at least one electroactive product.

A further aspect of the present disclosure related to an array comprising plurality of systems. Each of the systems comprise:
(a) a pathogen identification and/or quantification unit comprising a first electrode arrangement comprising at least one first working electrode located within at least one first chamber; and (b) a profiling unit comprising a second electrode arrangement comprising at least one second working electrode located within at least one second chamber. The second chamber is connected to the at least one first chamber to allow selective fluid transmission from the at least one first chamber to the at least one second chamber. It should be further noted that the at least one first working electrode of the identification/quantification unit of the system of the disclosed array is contacted directly or indirectly to at least one target binding site and/or moiety specific for binding one or more target pathogens. Still further, the at least one second working electrode of the profiling unit of the system of the disclosed array is in the vicinity of, or is connected directly or indirectly to at least one substrate molecule/s. The interaction of the at least one substrate molecule/s with at least one catalytic macromolecule, catalyzes the production of at least electroactive product.

A further aspect of the present disclosure relates to a method for identifying, quantifying and/or catalytically profiling one or more target pathogens in at least one sample. The disclosed method comprises the following steps:

First in step (a), contacting the sample or any preparation thereof, with at least one first electrode arrangement comprising at least one first working electrode, or with any unit or system thereof. The at least one first working electrode is connected directly or indirectly to at least one target binding site and/or moiety for binding and/or capturing one or more target pathogens from the sample. The next step (b), involves performing an electrochemical impedance spectroscopy (EIS) analysis of said sample; wherein impedance variations indicate the presence and/or quantity of the target pathogen in the sample. Step (c), involves applying disruption conditions to a target pathogen bound to, or captured by, the target binding site and/or moiety thereby generating target pathogen lysates. The next step (d), concerns contacting the target pathogen lysates with one or more substrate molecules, that may be in some embodiments connected directly or indirectly to the working electrodes, or alternatively or additionally, are in the vicinity of one or more second working electrodes, of the at least one second electrode arrangement, or with any unit or system thereof. According to such embodiments, the substrate molecules may be contained in the second chamber, and once the pathogen lysates are introduced to the sample, catalytic macromolecule/s that may exist in these lysates may act on the substrate to produce detectable electroactive products that are detected by the at least one second electrode/s. The at least one substrate molecule suitable for the disclosed methods is a substrate of at least one catalytic macromolecule. The catalytic macromolecule catalyzes the formation of at least one electroactive product using the substrate. In some embodiments, the catalytic macromolecule (e.g., enzyme) in the sample may catalyze the conversion of the substrate molecule to form at least one electroactive product. The next step (e) involves performing an electrochemical voltammetry or amperometry analysis of the sample to detect the production of at least one electroactive product. It should be understood that the detection of the product indicates the presence and/or activity of the catalytic macromolecule in the target pathogen lysates, thereby identifying and/or profiling the catalytic activity of the target pathogen in the sample.

A further aspect of the present disclosure relates to a diagnostic method. More specifically, provided herein is a method for diagnosing an infectious disease caused by at least one pathogen in a subject. The method comprises the step of detecting, identifying, quantifying and/or catalytically profiling one or more target pathogens in at least one sample of the subject. More specifically, the method comprising the following steps: In step (a), contacting the sample with at least one first electrode arrangement comprising at least one first working electrode, or with any unit or system thereof. The at least one first working electrode is connected directly or indirectly to at least one target binding site and/or moiety for binding and/or capturing one or more target pathogens present in the sample. Step (b) of the disclosed methods involves performing an electrochemical impedance spectroscopy (EIS) analysis of the sample. It should be understood that impedance variations, indicate the presence and/or quantity of the target pathogen in said sample. Step (c) involves applying disruption conditions to a target pathogen bound to, or captured by, the target binding site and/or moiety thereby generating target pathogen lysates. Next, in step (d), contacting the target pathogen lysates with one or more substrate molecules connected directly or indirectly to, or in the vicinity of, one or more second working electrodes of at least one second electrode arrangement, or with any unit or system thereof. It should be noted that the at least one substrate molecule is a substrate of at least one catalytic macromolecule that may be found in the sample. More specifically, the catalytic macromolecule catalyzes the formation of at least one electroactive product using the substrate. In some embodiments, the catalytic macromolecule may catalyze the conversion of the substrate to form at least one electroactive product. Step (e), involves performing an electrochemical voltammetry or amperometry analysis of the sample to detect the production of at least one electroactive product. It should be understood that the detection of the product indicates the presence and/or activity of the catalytic macromolecule in the target pathogen lysates.

The disclosed method thereby provides diagnosis of an infectious disease caused by at least one pathogen in the subject, identification and/or quantification of the pathogen and/or profiling the catalytic activity of the pathogen in the subject.

A further aspect of the present disclosure relates to a method of treating, preventing, ameliorating, reducing, or delaying the onset of an infection by at least one bacterial strain expressing at least one T3SS in a subject in need thereof. The disclosed therapeutic methods involve the diagnostic step as discussed above, together with profiling any pathogen that may exist in the sample, by providing valuable information with respect to the presence and/or activity of catalytic macromolecules that may be expressed by or associated with the pathogen in the sample. This information enables the evaluation of the pathogenicity of the pathogen and provides effective treatment regimen. Thus, in some embodiments, the therapeutic methods provided herein comprise:

First (a), classifying a subject as a subject infected by a bacterial pathogen if the presence of at least one T3SS component is determined in at least one sample of the subject. The second step (b), involves determining the antibiotic resistance profile of the bacterial pathogen in a sample of the subject. The determination of the presence of the at least one T3SS component in the sample, and profiling the antibiotic resistance of the bacteria is performed by the steps of: First (i), contacting at least one sample of the subject with at least one first electrode arrangement comprising at least one first working electrode, or with any unit or system thereof. The at least one first working electrode is connected directly or indirectly to at least one target binding site and/or moiety for binding and/or capturing one or more target pathogens from the sample. Next (ii), performing an EIS analysis of the sample. It should be noted that impedance variations indicate the presence and/or quantity of the target pathogen in the sample. The next step (iii), involves applying disruption conditions to a target pathogen bound to, or captured by, the target binding site and/or moiety thereby generating target pathogen lysates. The resulting target pathogen lysates in the next step (iv), are contacted with one or more substrate molecules connected directly or indirectly to one or more second working electrodes of at least one second electrode arrangement, or with any unit or system thereof. The at least one substrate molecule is a substrate of at least one enzyme providing antibiotic resistance to the pathogen. Still further, in some embodiments, the enzyme catalyzes the formation of at least one electroactive product using the substrate. In some specific embodiments, the enzyme catalyzes the conversion of the substrate molecule to form at least one electroactive product. The next step (v) involves performing an electrochemical voltammetry or amperometry analysis of the sample to detect the production of at least one electroactive product. The detection of the product indicates the presence and/or activity of the enzyme in the target pathogen lysates, thereby profiling the antibiotic resistance of the target pathogen in the sample. The next step (c), of the therapeutic methods involves administering to a subject classified as an infected subject in step (a), a therapeutically effective amount of at least one anti-bacterial agent, in accordance with the antibiotic resistance profile determined in step (b).

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B. The dual identification, quantification and characterization system

FIG. 1A. illustrates the dual system operating for capturing a selected pathogen within an identification unit.

FIG. 1B. illustrates the dual system operating for profiling and characterization of the selected pathogen.

FIG. 2. is a flow diagram showing sample analysis according to some embodiments of the present disclosure.

FIG. 3A-3B. Flow diagram for sample analysis according to some embodiments of the present disclosure

FIG. 3A. exemplifies operations associated with the identification unit.

FIG. 3B. exemplifies operations associated with the profiling unit.

FIG. 4. Dual-mode electrochemical detection and characterization system

The figure illustrates a system composed of two units or channels. The first unit, indicated as Channel 1, provides detection, quantification and capturing of a target pathogen in a sample. The unit is composed of electrochemical biochips, each configured as an individual electrochemical cell array equipped with a multiplicity of microelectrodes and is based on the use of a target-recognition moiety (e.g., antibody) attached to the working electrodes. The microelectrodes are individually addressable and multiple channels can be interrogated simultaneously. The second unit, indicated as Channel 2, provides characterization of the captured pathogen, specifically, enzymatic profiling and is based on a substrate of an enzyme attached to the working electrodes. The platform also contains a miniaturized ultrasonic transducer, a filtering unit, a waste chamber, and a potentiostat circuit. The system may contain contact pads to interface with a USB-stick potentiostat (left). The potentiostat circuitry and biochip microelectrodes are amenable for miniaturization and compatible with CMOS-integrating technologies potentially allowing for a massively multiplexed detection, low power consumption and cloud analysis via smartphone-enabled data acquisition.

FIG. 5A-5C. Schematic representation of construction of a miniaturized electrochemical dual detection biosensor

FIG. 5A. Impedimetric biochip channel for EspB detection,

FIG. 5B-5C. DPV channel for β-lactam resistance detection using nitrocefin assay (FIG. 5C).

FIG. 6A-6B. Characterization of the reference electrode

FIG. 6A. Following fabrication (and surface characterization of the deposited electrodes), the reference electrodes are electroplated. The electroplating of silver yields a typical white luster deposit that appears, in a SEM analysis, as a homogenous crystalline deposit with dense Ag nuclei of ˜1 μm (Bar: 5 μm).

FIG. 6B. Verification of a newly formed Ag/AgCl reference electrode is carried out by measuring its potential versus a commercial reference electrode in varying electrolyte (KCl) concentrations. The response of the electrode is plotted against the log[KCl] such that any log change in KCl concentration is expected to yield a 59 mV potential difference, according to the Nernst equation. In practice, deviations from this value are expected to evolve from the nature of the measured electrode (an ‘open’ reference electrode), the quality differences, and experimental conditions (mainly varying distances between the measuring electrodes that affect solution resistance). The reference electrodes demonstrate a ‘Nernstian behavior’, close to the theoretical value. measurements were performed in triplicates. Error bars denote SD from the mean.

FIG. 7A-7C. Characterization of the EC cell

Verification of the whole cell is obtained cyclic voltammetry with the well-known redox couple ferricyanide.

FIG. 7A. shows CV at different scan rates with a solution of 20 mM ferricyanide/ferrocyanide. Four different scan rates were used consecutively.

FIG. 7B. Corresponding analysis obtained from the biochip. The peak height increased as scan rate increased and was linearly proportional to the square root of the scan rate, showing the anodic peaks (top) and cathodic (bottom).

FIG. 7C. peak separation is relatively independent of scan rate. Error bars are the SD from the mean for triplicates.

FIG. 8A-8B. Biofunctionalization of EC chips

FIG. 8A. Immobilization of antibodies is based on covalent attachment using well-established gold-thiol chemistry. Antibodies were thiolated using the thiolating reagent 2-imminothiolane hydrochloride (Traut's reagent), which reacts with primary amines (—NH₂) to introduce sulfhydryl (—SH) groups while maintaining charge properties similar to the original amino group. The reaction was optimized to obtain an average of ˜6 —SH group per antibody. FIG. 8B. Ellman assay using DTNB (left) was used to assess the thiolation efficiency. The reaction is monitored by a spectrophotometer.

FIG. 9A-9F. Surface characterization of functionalized electrodes

FIG. 9A-9D. Assessment of thiolated antibodies immobilization to the gold working electrode is carried out by fluorescence microscopy analysis. Thiolated Cy3-labeled antibody is incubated on the gold WE. As a control, a non-thiolated Cy3 antibody was used. Incubation is followed by rigorous rinsing of the electrodes.

FIG. 9E-9F. AFM image of gold working electrode surface before and after the covalent immobilization of thiol-modified antibodies.

FIG. 10A-10E. mAb-EspB-B7-based impedimetric biosensor Figure shows schematically illustrates a biosensor (e.g., mAb-EspB-B7-based impedimetric biosensor), and cell suspension measurement conducted therewith, according to some possible embodiments.

FIG. 10A. demonstrates EIS-based detection of whole bacterial EPEC cells. In this non-limiting example electrochemical chips (with a working electrode e_{w} radius of about 0.3 mm, counter electrode e_{c} having radius of about 0.6 mm, and a square reference electrode eᵣ having surface area of about 0.25 mm², and respective contact pads 13w,13c,13r electrically connecting thereto) fabricated in/on a substrate (13) using microelectronic fabrication technologies and are subsequently modified with a thiolated mAb-EspB-B7 using thiol-gold chemistry. The electrodes e_{w},eᵣ,e_{c} are sealably enclosed inside an electrochemical cell structure, configured to receive a sample. The immobilization of mAb-EspB-B7 and capture of antigen affect the impedance measured between the underlying electrodes. An EIS measurement thus allow for the interrogation of the electrochemical system and separation of the individual components that affect the electrochemical cell circuit established by introducing the sample into the electrochemical cell (ci). The generated Nyquist plot is fitted to an equivalent circuit from which the different resistance values are extracted (inset).

FIG. 10B. Shows the Nyquist plots obtained from measurements of a bare gold working electrode (bare GE), from the working electrode after the immobilization of mAb-EspB-B7 (GE+mAb) thereon, and the mAb-EspB-B7-coated working electrode after incubation with 250 μg/mL purified EspB protein (GE+mAb+EspB).

FIG. 10C. Shows relative R_{ct} (charge transfer resistance) values of purified EspB protein (1, 4, 10 and 250 μg/ml) demonstrating a dose-dependent increase in the detected R_{ct} values. Relative R_{ct} values are the means of the R_{ct} ratios (before and after antigen capture) calculated from 3-6 measurements. Error bars represent the ±SD.

FIG. 10D. Shows that the change in the detected R_{ct} values is exponentially dependent on EspB concentration.

In FIG. 10E. specific binding of WT EPEC cells is indicated, resulting in a larger contribution to R_{ct} compared with the ΔespB null strain. The percent change in R_{ct} ratios measured for EPEC WT and ΔespB was calculated and averaged from 20 repeats (five measurements each containing four samples) for each strain. The mean of the averaged ratios and the standard error of the mean were calculated.

FIG. 11A-11B. Immuno-impedimetric detection of EspB antigen

FIG. 11A. IES-Nyquist plots. The Nyquist plots obtained from measurements of a bare gold electrode (bare GE), electrode modified with SAM (GE-SAM), electrode immobilized with mAb-EspB-B7 (GE-SAM-mAb), and after specific binding of purified EspB protein (GE-SAM-mAb-EspB).

FIG. 11B. EspB antigen detection calibration curve prepared by incubating GE-SAM-mAb with different concentrations of EspB antigen (0, 0.001, 0.01, 0.1, 1.0, 10 and 100 μg/mL). Error bar indicates SEM, n=9.

FIG. 12A-12B. Immuno-impedimetric detection of EspB-presenting whole bacteria FIG. 12A. Detection of EspB producing bacteria. The Nyquist plots from measurements of a bare gold electrode (bare GE), electrode modified with SAM (GE-SAM), electrode immobilized with mAb-EspB7 (GE-SAM-mAb), and after specific binding of EspB-presenting bacteria strains (WT-Sm^{r}, ΔespB-NA^{r}, WT-Tet^{r} and ΔespB-Tet^{r}) with electrochemical biochips.

FIG. 12B. Detection of EspB producing bacteria. The bar graphs represent the relative (%) change in charge transfer resistance (R_{ct}) obtained by the EspB-presenting bacteria strains (WT-Sm^{r} and WT-Tet^{r}, marked in the figure as WT-SM, WT-Tet, respectively) and control strains (ΔespB-NA and ΔespB− Tet^{r}, marked in the figure as EPEC-NA and EPEC-Tet) that do not express EspB. The recorded background signals are likely a result of non-specific adsorption. The significant change in R_{ct} values, obtained by the WT-SM strain, indicates efficient binding of EspB-presenting bacteria cells captured on the biochip. In comparison, a lower number of WT-Tet cells were seemingly captured by the biochip, suggesting a lower EspB expression by WT-Tet. Error bar indicates SEM, n=9.

FIG. 13A-13B. Optimization of colorimetric detection of β-lactamase

FIG. 13A. UV-Visible spectra of nitrocefin assay with different concentrations of β-lactamase (0-100 ng/mL).

FIG. 13B. Standard calibration curve for nitrocefin assay. Error bar represent standard error of mean (n=9 repeats).

FIG. 14A-14B. Colorimetric nitrocefin assay for the detection of β-lactamase from cultured bacterial strains cell lysates FIG. 14A. UV-Visible spectra of nitrocefin assay with different bacteria cell lysate and their absorbance intensities at 490 nm summarized in a bar graph.

FIG. 14B. Error bar represent standard error of mean (n=9 repeats).

FIG. 15A-15B. Electrochemical analysis of Nitrocefin assay with purified β-lactamase

FIG. 15A. shows CV analysis for nitrocefin substrate before hydrolysis (control) and after hydrolysis by β-lactamase.

FIG. 15B. shows SWV for nitrocefin substrate before hydrolysis (control) and after hydrolysis by β-lactamase.

FIG. 16A-16B. Optimization of DPV-based biosensor for the detection of β-lactamase activity

FIG. 16A. Nitrocefin assay with different concentrations of beta-lactamase. voltammograms of hydrolyzed nitrocefin at different concentration of β-lactamase (0-100 ng/mL).

FIG. 16B. Peak current vs conc. Of beta-lactamase. Standard electrochemical calibration curve showing the dependence of hydrolyzed nitrocefin oxidation on β-lactamase concentrations. Error bar represent standard error of mean (n=9 repeats).

FIG. 17A-17B. Electrochemical detection of nitrocefin hydrolysis by β-lactamase enzyme in bacterial samples

The figure shows electrochemical detection of nitrocefin hydrolysis by bacterial samples with β-lactamase enzyme [WT EPEC pCX341 NleD-β-lactamase (Tet^{R}) (marked in the figure as WT-Tet); EPEC ΔespB pCX341 NleD− β-lactamase (Tet^{R}) (marked in the figure as EPEC-Tet) or without; WT EPEC (Sm^{R}) (marked in the figure as WT-SM); and EPEC ΔespB(NA^{R}) (marked in the figure as EPEC −NA)], as indicated in the figure.

FIG. 17A. shows an SWV analysis of nitrocefin assay with bacteria cell lysates from antibiotic resistant and wild type bacteria.

FIG. 17B. Shows a bar graph for measuring current signal potential at 1.02V for all the bacteria cell lysates with nitrocefin. Error bars represent the standard deviation of means for triplicate.

FIG. 18A-18B. DPV-based biosensor for the detection of β-lactamase in different bacteria strains cell lysates

FIG. 18A. Voltammograms of hydrolyzed nitrocefin with different cell lysates.

FIG. 18B. The average measured peak current at a potential of +0.92V recorded from the different strains. The error bar represents standard error of mean (n=9 repeats).

FIG. 19A-19D. DPV biosensor for the detection of β-lactamase producing bacterial strains

FIG. 19A. Optical detection of β-lactamase producing bacteria obtained from EIS chips.

FIG. 19B. Bar graph represents the measured optical absorbance intensity at 490 nm from nitrocefin assay with bacterial cell lysates.

FIG. 19C. DPV-based Electrochemical detection of β-lactamase producing bacterial strain WT EPEC pCX341 NleD-β-lactamase (Tet^{R}) (marked as WT-Tet) with the controls EPEC ΔespB pCX341 NleD− β-lactamase (Tet^{R}), WT EPEC (Sm^{R}), and EPEC ΔespB(NA^{R}) (marked as EPEC-Tet, WT-SM, EPEC-NA, respectively).

FIG. 19D. Bar graph indicates the measured peak current at +0.92V of nitrocefin assay with β-Lactamase producing bacterial strains WT EPEC pCX341 NleD-β-lactamase (Tet^{R}) (marked as WT-Tet) with the controls EPEC ΔespB pCX341 NleD− β-lactamase (Tet^{R}), WT EPEC (Sm^{R}), and EPEC ΔespB(NA^{R}). Error bar represents SEM, n=9 (marked as EPEC-Tet, WT-SM, EPEC-NA, respectively).

### DETAILED DESCRIPTION OF THE INVENTION

Before specific aspects and embodiments of the invention are described in detail, it is to be understood that the present disclosure is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

The present disclosure describes a selected application of a dual-mode electrochemical platform that contains two electrochemical biochips, each configured as an individual electrochemical cell array equipped with a multiplicity of microelectrodes. In some embodiments, the electrochemical chips, which may be in some embodiments, enclosed in separate chambers, are connected by a microfluidic channel enabling a sequential measurement of a sample. The platform also contains a miniaturized ultrasonic transducer, a filtering unit, a waste chamber, and a potentiostat circuit. This platform was applied in the detection of at least one pathogen, specifically, EspB-presenting EPEC that also possesses antibiotic resistance by expressing ESBL enzyme (extended spectrum β-lactamase). Thus, the systems and platform of the present disclosure provide quantitative determination of two biomarkers, the identity and quantity of a pathogen in the sample, as well as profile of the enzymatic activity of the pathogen, and specifically, the antibiotic resistance of such pathogen.

For identifying and quantifying the pathogen in a sample, specifically, T3SS pathogen, the present disclosure uses a specific mAb raised against EspB, an essential component within the T3SS that is crucial for the infectivity of numerous Gram-negative bacteria, including EPEC. The present disclosure further combines the use of this antibody for pathogen identification, with functional measurements to further characterize the pathogen present in the sample. For example, by profiling the antibiotic resistance of the pathogen.

The results presented by the present disclosure show that the combination of T3SS detection with determination of drug resistance improves sensitivity and provides not only the diagnostic information with respect to the pathogen identity and/or quantity, but also therapeutic information with respect to drug sensitivity and resistance. The platform, systems and methods disclosed herein provide therefore a personalized approach for combined diagnostic and determination of treatment regimen for a tested subject.

In a first aspect, the present disclosure provides a system. In some embodiments, the disclosed system is configured to capture one or more selected pathogen and to analyze parameters of the captured pathogen. The system comprises an identification unit configured for identifying and capturing one or more pathogens from a biological sample, and a profiling unit configured for profiling and characterizing the one or more pathogens to provide data indicative thereof. The identification unit is connectable to the profiling unit to selectively enable selective transmission of fluids from the identification unit to the profiling unit.

The identification unit comprises at least a first chamber and at least a first electrode arrangement positioned such that an active portion of the electrode arrangement is located within the chamber. The first electrode arrangement comprises at least one working electrode connected directly or indirectly to target binding moiety for binding one or more pathogens. Additionally, the identification unit comprises or associated with a disruption unit (disruptor) configured to apply a selected disruption conditions on materials in the first chamber. The disruption conditions may be physical, mechanical and/or chemical disruption conditions that cause lysis to biomaterial in the chamber, while preserving activity of macromolecules or enzymes of the biomaterial or contained in the biomaterial or associated with the biomaterial. In some embodiments, the biomaterial is, or derived from the pathogen captured in the first pathogen identification unit. In some examples, the disruptor may be an ultrasound generator, e.g., transducer, directing ultra-sonic vibrations on materials in the first chamber.

The profiling unit comprises at least a second chamber, and a respective second electrode arrangement positioned such that active portions of the electrode arrangement are located within the second chamber. The second electrode arrangement comprises one or more working electrodes connected directly or indirectly to one or more substrate materials. The substrate materials are selected to interact with one or more macromolecules or enzymes in pathogen lysates to form at least one product. In some embodiments, the resulting product is formed by one or more macromolecules or enzymes in the lysates of the captured pathogen, using the substrate. In some embodiments, the product produced is a modified substrate material, and/or a substrate that is converted by an enzymatic activity of a macromolecule in the pathogen lysates to form a detectable product, for example, at least one electroactive product.

Generally, in some embodiments, the first electrode arrangement is connectable to a detection circuit adapted for electrical detection of pathogen attached to the target binding moiety. The detection circuit may be adapted to perform electrochemical impedance spectrometry measurements to thereby detect and quantify one or more pathogens captured by the target binding moiety.

Further, in some embodiments, the second electrode arrangement is connectable to a voltametric circuit adapted to perform voltametric measurement between electrodes of the second electrode arrangement. To this end, in some embodiments, the one or more substrate materials may be selected in accordance with electrical difference between modified and unmodified substrate material. Still further, in some embodiments, an appropriate substrate may be a substrate of a macromolecule that may be present in the pathogen, for example, a specific substrate for an enzyme that may be present in the detected pathogen. Still further, such macromolecule/s or specifically, enzyme/s may be associated with or connected directly or indirectly to the pathogenicity of the pathogen. Thus, in some embodiments, a suitable substrate may be a material that facilitate, and/or is a direct substrate, and/or is used by an enzyme (or any other macromolecules) in the pathogen lysate to form a detectable product, or a substate that can be specifically converted by the enzyme in the pathogen lysate into a detectable product. In some embodiments, an electroactive product. Particular substrates useful in the systems disclosed herein are described in more detail in connection with other aspects of the preset disclosure.

In some embodiments, the system may further comprise a controller connectable to one or more valves and said first and second electrode arrangement. The controller is configured and operable to indicate sample input to said identification to, operate said first electrode arrangement for detecting one or more pathogens attached to said target binding moiety and quantity of attached pathogens, operate a waste valve for rinsing out remains of said sample, inserting selected amount of water to said first identification chamber, operate said disruptor to apply disruption field to thereby generate pathogen lysates of said one or more pathogens, operate a transmission valve to transmit said pathogen lysates to said profiling unit, operate said second electrode arrangement for detecting level of modification to said one or more substrate materials, and provide output data indicative of activity profile of said one or more pathogens. Accordingly, the above-described detection circuit and/or profiling circuit may be formed as hardware or software modules of the controller.

It should be understood that in some embodiments the target binding moiety is attached, connected, comprised within, deposited, integrated into, printed onto the at least one working electrode. Thus, the working electrode in some embodiments, is connected to, attached to and/or carries at least one target binding moiety. Still further, in some embodiments, the target binding site and/or moiety may be connected directly to the working electrode, or alternatively, via at least one linker or any other linking moiety that may be any chemical entity or modification, or alternatively, any peptide linker or any other chemical linker, for example, a small molecule linker. In some embodiments, where the target binding moiety is an antibody, attaching the antibody (binding moiety), to the working electrode involves gold-thiol chemistry, specifically, attaching the thiolated antibody to the electrode. Still further, in some specific and non-limiting embodiments, antibodies were thiolated by using the thiolating reagent 2-imminothiolane hydrochloride (Traut's reagent), which reacts with primary amines (—NH₂) to introduce sulfhydryl (—SH) groups while maintaining charge properties similar to the original amino group. The reaction was optimized to obtain an average of ˜6 —SH group per antibody. Thus, in some embodiments, the working electrode is covalently attached to immobilized thiol-modified antibodies, that serves as a target binding moiety. In some embodiments, working electrodes surfaces may be first covered with for example 11-amino-undecanothiol to form a self-assembled monolayer (SAM) with free amine functional groups and then incubate with antibodies to form mAb-immobilized electrochemical chips.

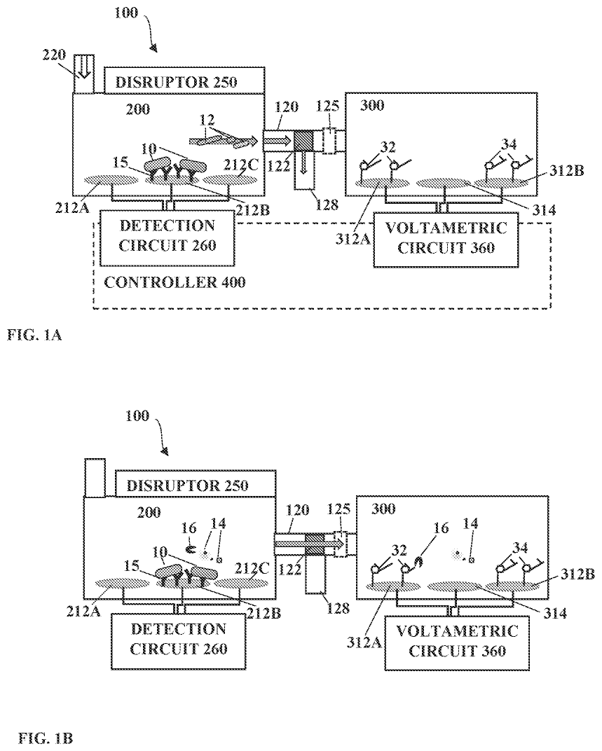

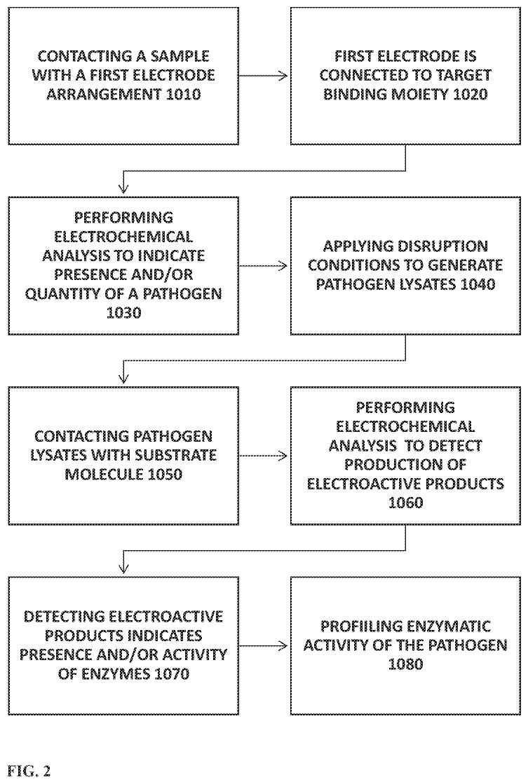

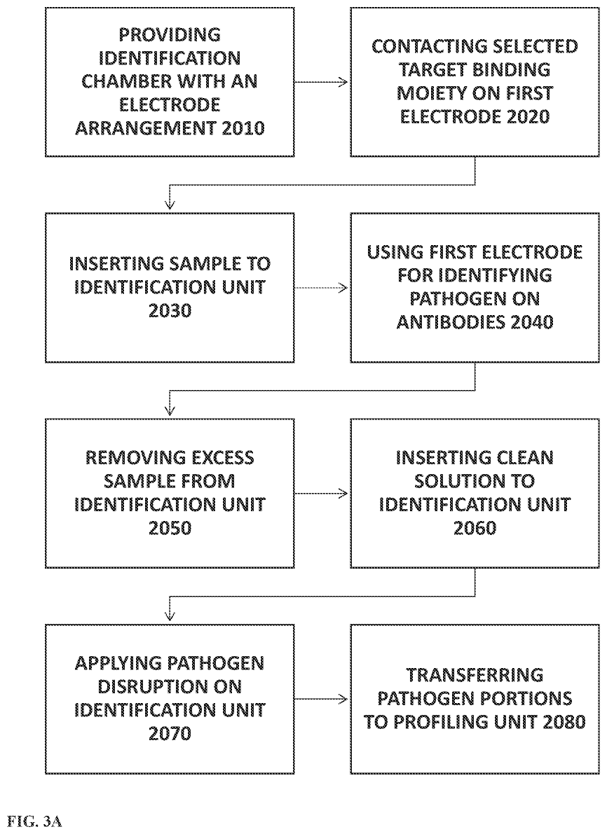

The present disclosure provides a system and methods for identifying and/or quantifying, as well as analyzing, characterizing and profiling pathogens present in the sample. For example, the present methods provide for detecting the presence, identifying and/or quantifying a specific pathogen in a sample, and for analyzing and characterizing the pathogen present in the sample by providing an enzymatic profile, specifically of one or more enzymatic properties of the identified pathogen. Enzymatic profiling of the pathogen in the sample, as provided by the disclosed methods and systems, comprises for example the provision of antibiotic resistance/sensitivity profiling of the pathogen. FIGS. 1A and 1B schematically illustrate a system 100 according to some embodiments of the present disclosure. FIG. 1A illustrates system 100 at a stage of identifying and/or quantifying and/or capturing one or more specific pathogen/s present in the tested sample, and FIG. 1B illustrates the system 100 at a stage of analyzing, characterizing and profiling the specific identified and captured pathogen present in the sample.

As illustrated in FIG. 1A, system 100 includes a first identification/quantification and/or capturing unit 200 and a second characterizing/profiling unit 300. System 100 may also include a controller 400 configured to operate certain functions of the system, as described in more detail below. The identification and profiling unit 200 and 300 include respective chambers for holding fluid sample, while selectively allowing fluid flow from a first chamber of the identification unit 200 to a second chamber of the profiling unit 300. More specifically, at least one channel 120 allows selective transfer of fluids from the first chamber of the identification unit 200 to the second chamber of the profiling unit 300. Generally, control valve 122 may be positioned along the at least one channel 120 to selectively prevent fluid transfer, direct fluid to waste port 128, or allow fluid transfer to profiling chamber 300. The channel 120 may also include one or more filters 125 positioned along fluid path to the profiling/characterization unit 300. Filter 125 may be selected to allow only materials smaller than a selected size (e.g., a range of about 100 KDalton or more to about 30 KDalton or less) to enable passage of macromolecules, for example proteins, and specifically, enzymes, while preventing transmission of the entire pathogen or large portion thereof, for example cell/s or other microorganisms (as specified by the preset disclosure), cell debris, or organelles of the specific identified pathogen. It should be noted that waste port 128 is illustrated herein as being a part of channel 120 as a non-limiting example. The waste port may be located at any selected region of the first chamber. Further, input port 220 described below may also be used for removing waste from the chamber.

Further, in some embodiments, the identification chamber may be associated with a disruptor 250 configured to apply selected disruption field/conditions (e.g., physical, mechanical, chemical) on materials in the chamber, the disruption field may be selected to cause lysis to biomaterial (e.g., the pathogen and/or any components or preparations thereof) in the chamber, while preserving the activity of the macromolecule/s therein e.g., preserve enzymatic activity. In this connection, the disruptor 250 may be a part of the identification unit 200 or a separate unit. The disruptor is configured and operable to apply disruption condition onto materials placed within the first chamber. For example, the disruptor 250 may be an ultrasonic transducer adapted for generating signals of ultrasonic frequency ranges and directing the signals toward the first chamber of the identification unit 200.

The identification unit 200 includes an electrode arrangement positioned with active ends of the electrodes located within the first chamber, being in contact with a sample when placed in the chamber and having electrical contacts for connecting to a respective detection circuit. The electrode arrangement includes one or more electrodes, including e.g., electrodes 212A, 212B and 212C generally configured as a working electrode, a reference electrode and a counter electrode respectively. The electrode arrangement is connectable or connected to a detection circuit 260 for performing selected detection measurements through the electrodes. The detection measurements may generally include impedance measurements enabling the detection of at least one pathogen 10 bound to the working electrode via a target binding moieties 15. To this end, at least one electrode, typically working electrode 212B is connected directly or indirectly to at least one specific target binding moiety 15 that specifically binds one or more selected pathogens.

The identification/quantification/capturing unit 200 may typically include an input sample port 220 configured to receive a sample, specifically, a liquid sample typically containing biological material. When such biological material is input to the first chamber of the identification unit 200, various pathogens that may be present in the sample may interact with the target binding moiety 15. In case that a pathogen 10 for which the target binding moiety 15 is specific is present in the sample, the pathogen 10 binds to the target binding moiety 15, while other biological material 12 can be removed from the chamber, optionally by rinsing toward a waste output port 128. As indicated above waste port 128 may be positioned at various locations of the chamber, for example waste port 128 may be a part of channel 120 as exemplified in FIG. 1A. In some configurations waste port may be common with input port 220 or located at other parts of the chamber.

FIG. 1B illustrates system operation following removal of undesired biological material through the optional waste port 128. At this stage, upon identification, quantification and capturing of the specific pathogen, the identification chamber 200 may be in some embodiments rinsed, and the disruptor 250 is operated to lyse the pathogen 10 left captured in the chamber. The disruption operation produces pathogen lysates that comprise macromolecules (e.g., proteins such as enzymes) present in, or associated with, the identified and captured pathogen 10. The pathogen lysates comprise various macromolecules, for example enzymes 14, and any other proteins or co factors 16. The pathogen lysates are transferred to the profiling unit 300 for characterization and profiling of the identified pathogen 10, based on lysates, and specifically macromolecules thereof.

To this end, profiling/characterization unit 300 includes at least one second chamber and an arrangement of one or more electrodes, illustrated by electrodes 312A, 312B and 314, connected to a voltametric circuit 360. The electrode arrangement is positioned with active ends located within the second chamber and may be connectable to the voltametric circuit 360. Generally, one or more of the electrodes may be configured as reference electrode 314, and one or more other electrode 312A and 312B may be configured as working electrodes. The working electrodes are connected directly or indirectly to selected one or more substrate molecules 32 and 34, for example, such that each electrode is connected to a respective substrate molecule/s 32 or 34. It should be understood that the profiling/characterization unit 300 of the disclosed system may comprise in some embodiments one or more, specifically, plurality of profiling (second) chambers, each containing a specific second electrode arrangement for a single substrate, or alternatively or additionally, a plurality of electrodes (each attached to, or positioned in the vicinity of, a single substrate molecule) in a single second chamber. The substrate molecules, for example 32 and 34, specifically interact with specific macromolecules respectively, for example with an enzyme that may be present, expressed by or associated with a pathogen identified in the sample. The specific interaction of the substrate in the profiling unit with certain enzyme/s or other protein/s, and/or other macromolecules in case present in the pathogen lysates (e.g., 14 and 16), and/or lead to the production of a detectable product by the enzyme and/or macromolecule in the lysates, using the substrate molecule. In yet some further embodiments, the macromolecule (e.g., enzyme) present in the pathogen lysates may modify, and/or convert the substrate molecule to form a detectable product, specifically, electro reactive product, or an electroactive modified substrate that is identifiable by voltametric measurement from the unmodified substrate. Suitable substrate molecules are those that produce or lead to the formation of, upon interaction thereof with at least one specific macromolecule (e.g., enzyme) in the sample, at least one electroactive product. The voltametric circuit 360 operates to apply selected one or more voltametric measurements (such as cyclic voltammetry, square wave voltammetry, differential pulse voltammetry etc.) on the electrodes 312A and 312B, typically/optionally with respect to reference electrode 314, to determine existence and quantity of modified substrate over the substrate molecules 32 and 34.

Generally, system 100 may also include a controller 400. The controller 400 may be connectable to the identification unit 200 and to the profiling unit 300 to operate the system as disclosed herein. The controller 400 may be an analog controller, or it may include one or more processors and memory circuitry carrying computer readable instructions for operating the system 100. To this end the controller may be connectable to one or more valves located along channel 120, input port 220 etc., and to said first and second electrode arrangement or the respective detection circuit 260 and voltametric circuit 360. The controller 400 may be configured and operable to be responsive to input signal indicating sample input to first chamber of the identification unit 200 to thereby initiate system operation. Accordingly, the controller may utilize a detection circuit 260 to operate the first electrode arrangement for detecting and/or quantifying one or more pathogens attached to target binding moiety. In response to data indicating capture of such pathogens, the controller may optionally operate a waste valve for rinsing remains of the sample out of the first chamber, leaving the captured pathogens in the first chamber. Rinsing the first chamber may optionally also include providing a selected (small) amount of water into the first chamber to provide aqueous conditions in the chamber. Further, the controller may operate disruptor 250 to apply disruption conditions in the first chamber, to thereby lyse the pathogen 10 left captured in the chamber, releasing pathogen lysates int first chamber. The controller may thus operate valve 122 to transfer the pathogen lysates 14 and 16 to the second chamber of the profiling unit 300. When the pathogen lysates are in the second chamber, and optionally following a selected interaction period, the controller may operate the second electrode arrangement to determine data on interaction of the pathogen lysates with the one or more substrate molecules 32 and 34. To this end the controller may apply selected voltage profiles between electrodes of the second electrode arrangement electrodes, e.g., electrodes 312A or 312B and 314, and collect data on current flowing therebetween. Generally, the controller may operate the electrode arrangement to apply voltametric measurement such as cyclic voltammetry, square wave voltammetry, differential pulse voltammetry and/or other suitable measurement, to determine presence and to quantify modification to the substrate molecules indicative of presence and quantity of respective macromolecules (enzymes proteins) in the pathogen lysates. The controller may thus generate output data indicative of detection and/or quantification of the specific pathogen/s and respective macromolecules detected in the pathogen lysates.

The controller may further include a user interface, e.g., including a display unit, keyboard, or any other input/output modules, and may also be connectable to a communication network. The one or more processors of the controller can be configured to execute several functional modules in accordance with computer readable instructions implemented on a computer readable medium. Operations associated with the controller may be implemented by respective hardware or software modules comprises in the one or more processor and memory thereof. Such functional modules, being implemented by hardware or software module are referred to herein as comprises in the controller.

The target binding moiety 15 placed in the identification unit 200, and the one or more substrate molecules 32 and 34 pleased in the profiling unit 300 provide identification, quantification and capturing, as well as characterization by profiling specific parameters of pathogens identified in the sample. For example, target binding moiety 15 may be an antibody specific for a pathogen, e.g., bacteria, enabling identification, quantification and capturing of a specific pathogen present in or associated with a biological sample. Alternatively, the target binding site or moiety may comprise any affinity molecule or any antigen binding protein, that specifically recognizes and binds the target pathogen. Further, the one or more substrate molecules 32 and 34 may include in some embodiments, substrate molecule/s specific for certain enzymes, for example, enzymes that render antibiotic resistance or any other virulent properties, that may be found or associated with the lysates of the identified and captured pathogen. For example, substrate molecule/s may include any substrate to an enzyme that provides enhanced virulence to the pathogen, for example, antibiotic resistance to the pathogen, or any enzyme that is associated with the formation of any toxic or virulent product of the pathogen. In some non-limiting embodiments, the substrate molecule is a substrate of an enzyme that provides antibiotic resistance to the pathogen. In some embodiments, the substrate is a substrate of at least one enzyme that provides directly or indirectly resistance to beta-lactam antibiotics, specifically, beta-lactamase. Specific and non-limiting embodiments for a suitable substrate for at least one beta-lactamase, may include Nitrocefin, that upon hydrolysis thereof by beta-lactamase, produces an electroactive product, thereby enabling detection of beta-lactamase presence in lysates of the identified pathogen. Such identification of beta-lactamase, or any antibiotic resistance enzyme or protein provides antibiotic-resistance profiling of the identified pathogen/s in the sample, and as such, therapeutic information that enabling accurate diagnosis and treatment. Thus, system 100, and the methods of the present disclosure enable analysis of a biological sample, specifically, detection, identification, quantification of specific pathogen/s in the sample, together with characterization, for example, enzymatic profiling (e.g., one or more enzymatic properties) of the identified pathogen present in the sample. This allows for detection and/or quantification of pathogens in a sample and further provides profiling of antibiotic resistance of each specific identified pathogen/s in the sample. The present methods and systems of the present disclosure therefore provide diagnostic and therapeutic information in a single step continuous platform.

FIG. 2 shows a flow diagram exemplifying a method for identifying and/or quantifying, as well as analyzing, characterizing, and profiling pathogens present in the sample according to some embodiments of the present disclosure. As shown, the method relates on contacting a sample with a first electrode arrangement 1010. The first electrode arrangement may be in a first chamber, allowing the sample to be in continuous contact therewith. The first electrode arrangement may include at least one first working electrode, being in contact directly or indirectly with a target binding moiety 1020 specific for binding one or more target pathogens. Generally, the target binding moiety may act to capture pathogen of a type, for which the target binding moiety is specific.

To determine capture of the pathogen, the method includes performing electrochemical analysis using the electrode arrangement 1030. The electrochemical analysis may include electrochemical impedance spectroscopy EIS, enabling to provide data on presence and/or quantity of captured pathogen. Optionally, in some embodiments, remains of the sample may be rinsed out of the chamber leaving the captured pathogen in the chamber. The method further includes applying disruption conditions on the captured pathogen 1040 to lyse the pathogen, thereby forming pathogen lysates. For analysis, the pathogen lysates may be transferred to a second chamber, to be contacted with substrate molecules 1050. The substrate molecules specifically interact with specific macromolecules that may be found in the pathogen lysates producing electroactive product. For example, the substrate molecules may interact with enzymes or proteins of the pathogen. Detection of the interaction may be provided by performing electrochemical analysis to detect the electroactive products 1060. To this end, the substrate molecules may be in contact with a second electrode arrangement, enabling voltametric and/or amperometry measurements to identify presence and/or quantity of the electroactive product. By detecting the electroactive products 1070, the method of the present disclosure can provide data on enzymatic activity of the pathogen 1080.

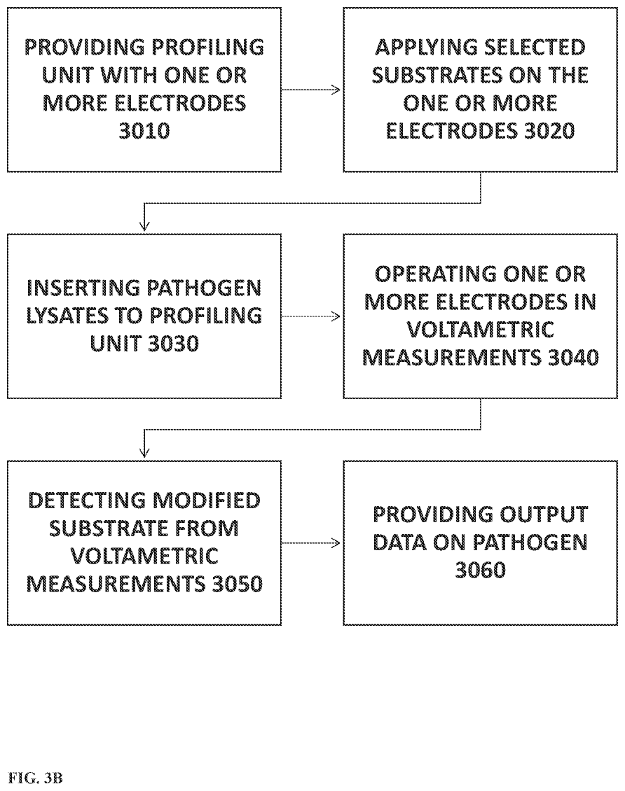

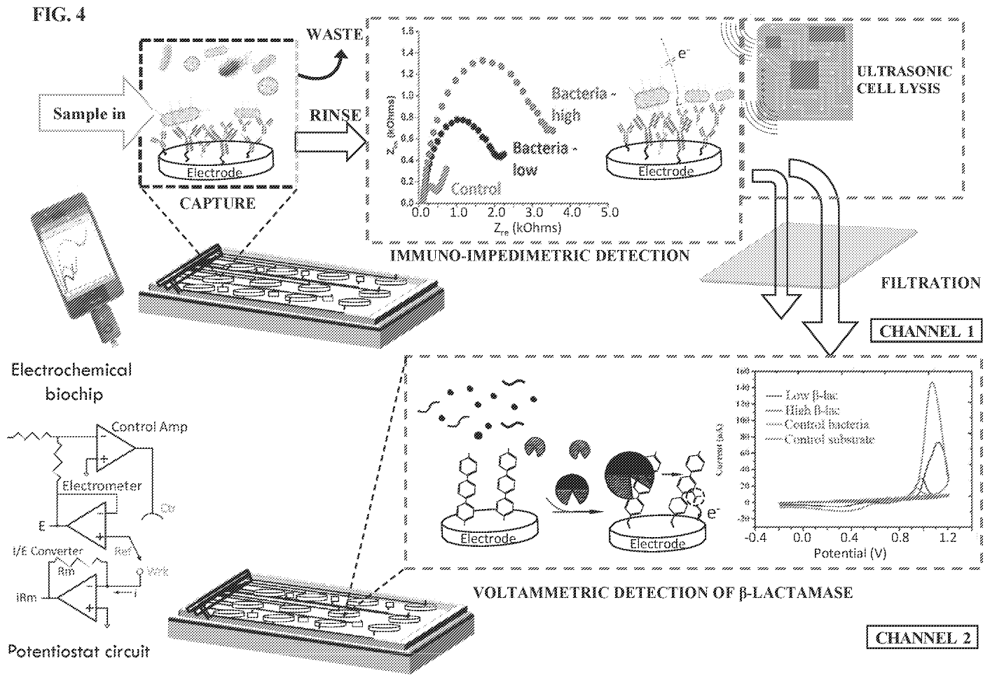

FIGS. 3A and 3B disclose flow diagrams exemplifying additional details for sample analysis according to some embodiments of the present disclosure. FIG. 3A exemplifies operation of the procedure within the identification/quantification/capturing unit, and FIG. 3B exemplifies operation of the procedure in the profiling/characterization unit. As shown, the present procedure includes the use of identification/quantification/capturing unit including at least a first chamber provided with an electrode arrangement 2010. To provide pathogen selectivity, at least a first electrode is contacted with specific target binding moiety 2020. The target binding moiety may be a specific antibody having specificity to one or more pathogens to be identified/quantified/captured. For processing a sample, the sample is inserted into the identification unit 2030. At this stage, pathogens for which the target binding moiety is specific bind thereto, while other biological material may remain within the fluid of the sample. To determine that the specific pathogen is bound to the target binding moiety, the electrode arrangement may be used for detecting selected electrical properties between the electrodes 2040. For example, the use of electrochemical impedance spectroscopy (EIS) can provide data on impedance between different electrodes of the electrode arrangement, and thus indicate existence, and quantity of pathogen/s bound to the target binding moiety of the working electrode. The EIS measurement/analysis of the sample may utilize at least one of faradic or non-faradic EIS analysis in accordance with amount of electroactive molecules/substrate within the first chamber. More specifically, in samples that contain high concentration of electroactive substrate, the EIS may utilize faradic EIS. Alternatively, in samples that do not contain sufficient concentration of electroactive substrate the measured impedimetric signal may reflect the charge transfer resistance of redox reactions of substrate in the sample or otherwise the changes in non-faradic currents. Given that presence of a sufficient amount of pathogen is detected on the working electrode, the excess sample fluid, including biological material other than the specific pathogen, is removed from the first chamber 2050 for the identification unit, and the chamber may be optionally rinsed with clean solution 2060. This provides that the specific pathogen is generally the only biological material present in the first chamber. The procedure further includes operating a disruption unit to apply disruption conditions (e.g., mechanical, chemical and/or other physical conditions) on the first chamber of the identification unit and the pathogen therein 2070. In some embodiments, the disruption conditions are selected to cause lysis of the pathogen, e.g., by mechanical disruption of membranes, organelles thereof, while maintaining the activity of macromolecules thereof, specifically, maintaining an intact enzymatic activity of pathogen enzymes, leaving the first chamber with pathogen lysates. The lysates or any macromolecules thereof are generally not captured/bound to the target binding moiety at this stage and can be transferred to the profiling unit 2080 by allowing fluid flow between the first and second chambers. Generally, the sample may be filtered when transferred between the chambers.

FIG. 3B exemplifies operations taking place in the profiling chamber. As indicated above, the profiling unit is prepared with one or more electrodes 3010, and selected electrodes are contacted directly or indirectly with specific substrate molecule/s 3020. Generally, at least one electrode is left clear to provide a reference electrode, while one or more other electrodes are contacted with specific one or more substrate molecules, such that each electrode is associated with a single substrate molecule. The substrate molecules are selected as substrate molecules that allow voltametric or amperometric detection between the substrate and a product thereof or a modified substrate produced due to interaction with one or more enzymes or other active agents (macromolecules) that may be present in pathogen lysates. Following the process of FIG. 3A, the sample containing pathogen lysates is inserted/transferred into the profiling/characterization unit 3030. The lysates may include various enzymes or any other molecules or macromolecules that may interact with the one or more substrates, generating a respective electroactive product or electroactive modified substrate. The technique utilizes operation of one or more voltametric or amperometry measurements 3040 between the one or more electrodes carrying respective substrates and the reference electrode. The measurements provide output voltametric data that enables detection of modified substrate therefrom 3050. Thus, detection of electroactive product and/or electroactive modified substrate is indicative of presence of respective one or more macromolecules, specifically, enzymes, or enzymatic activity, in the pathogen identified and captured in the identification chamber.

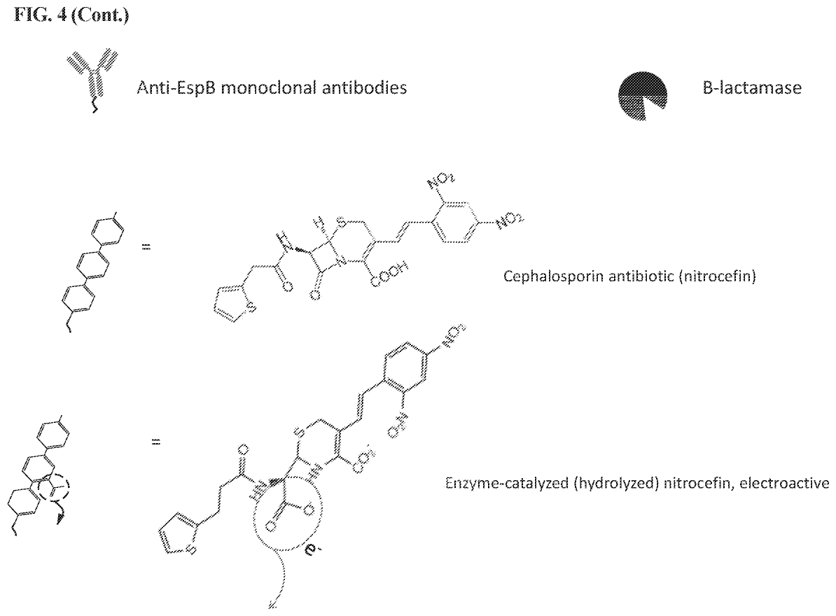

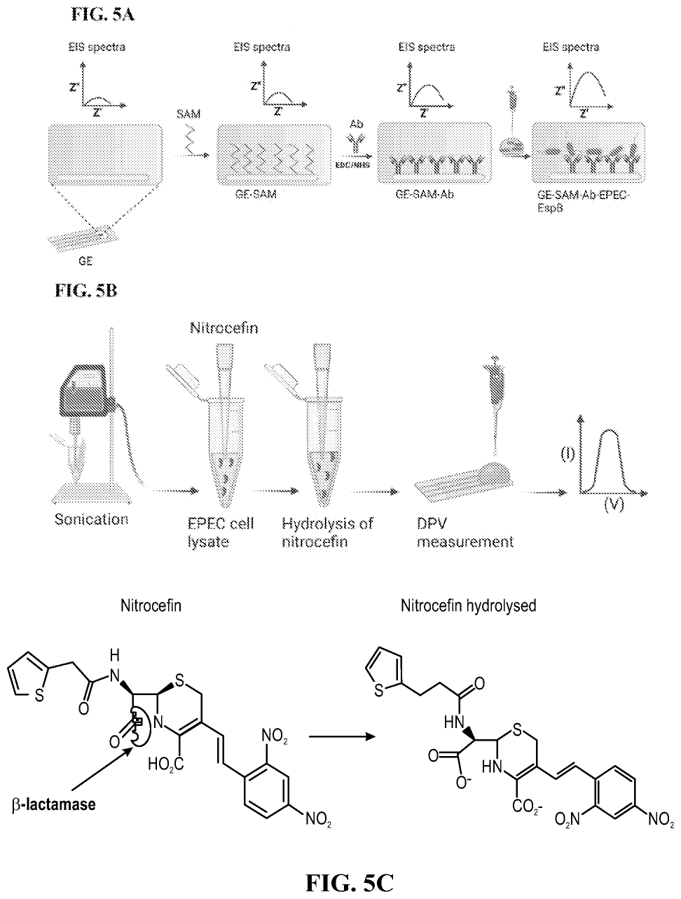

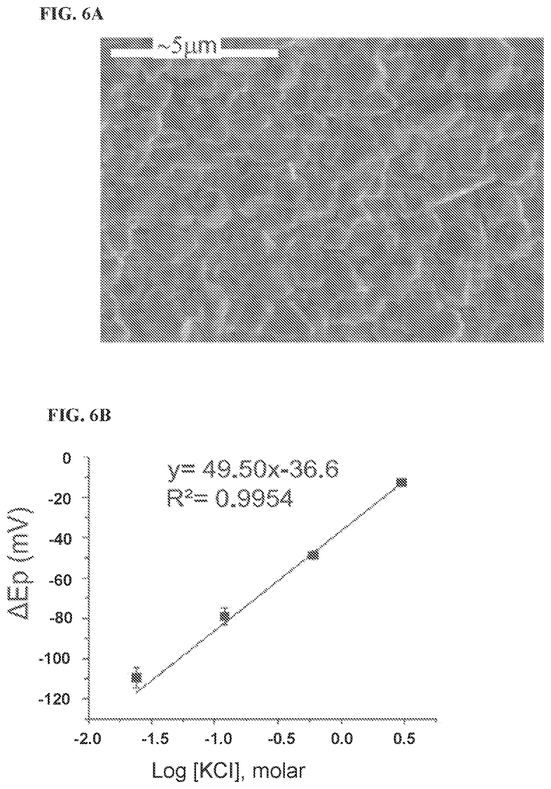

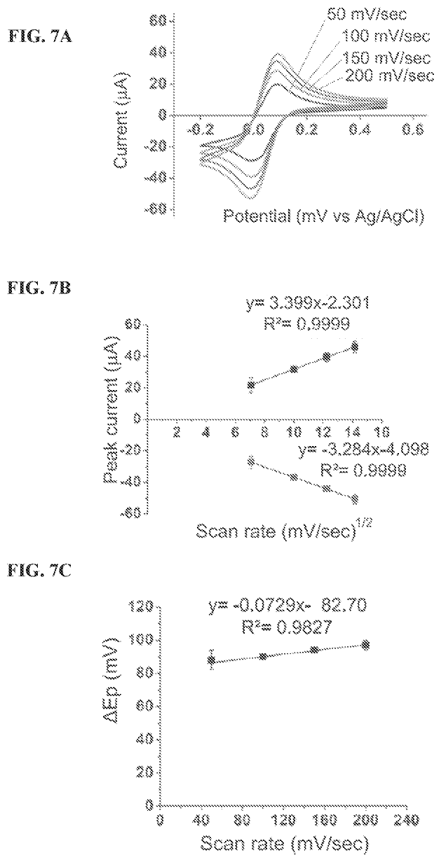

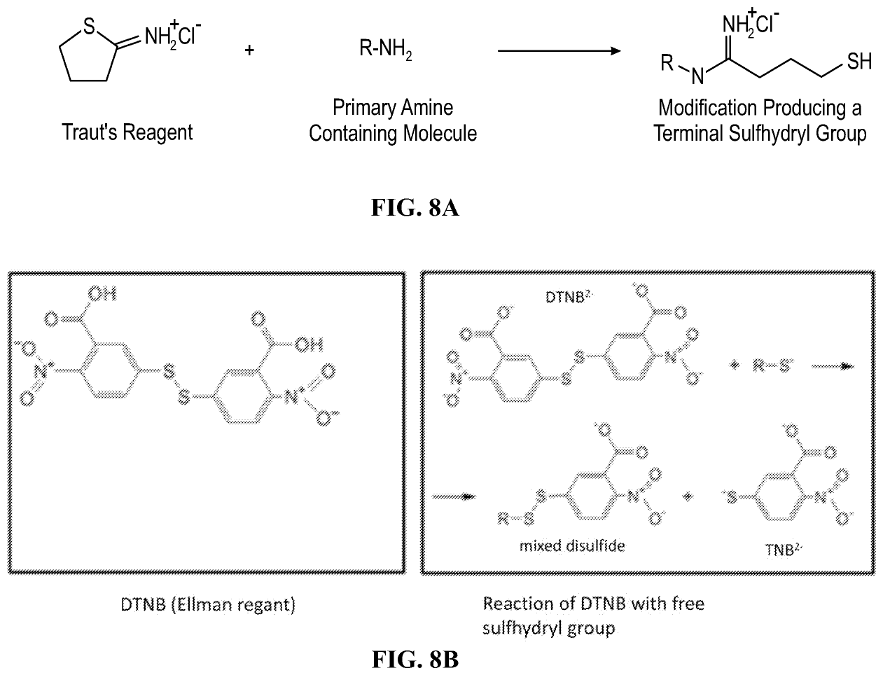

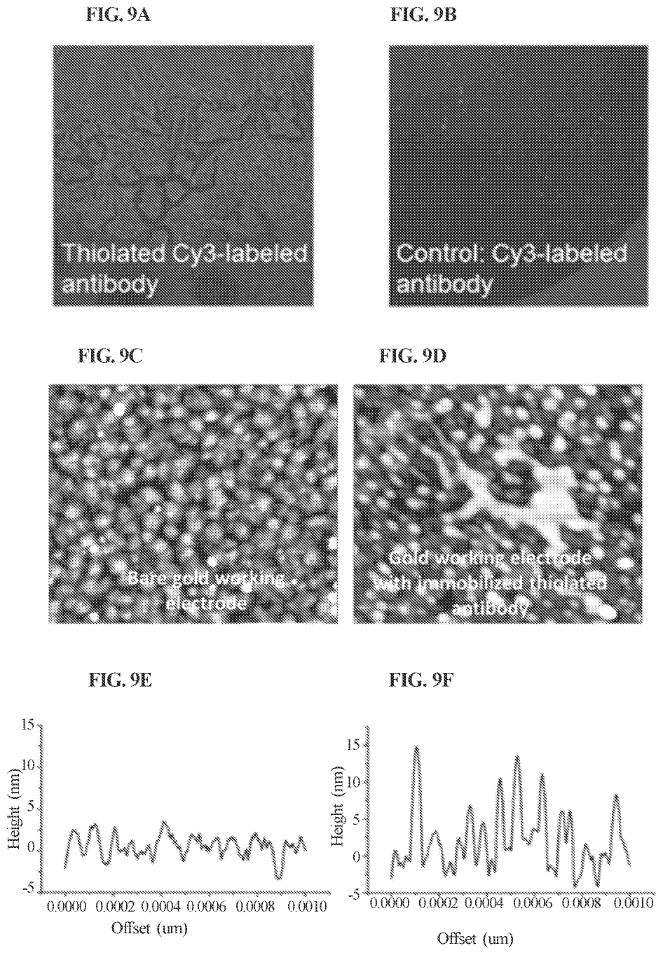

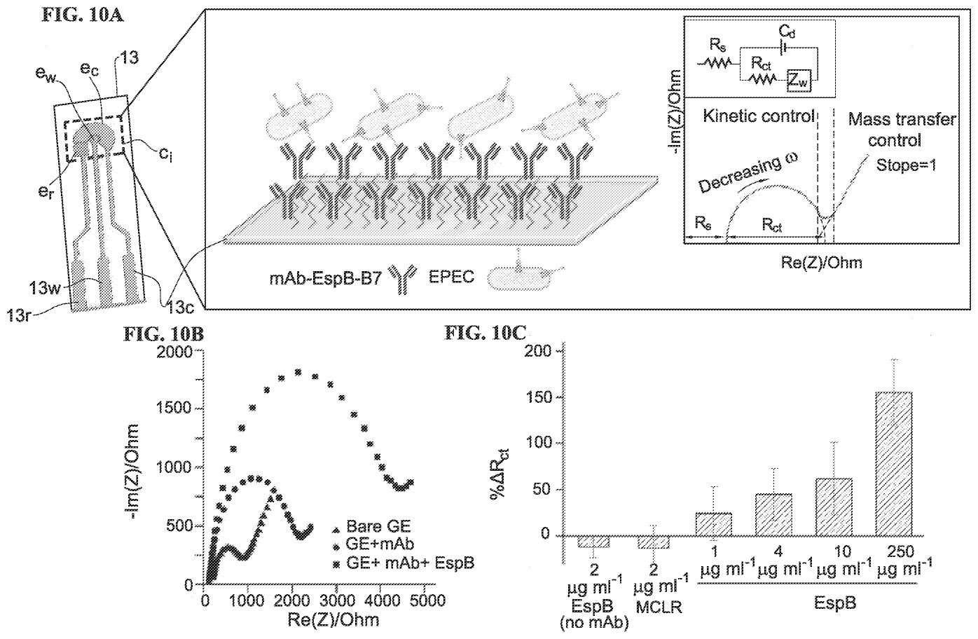

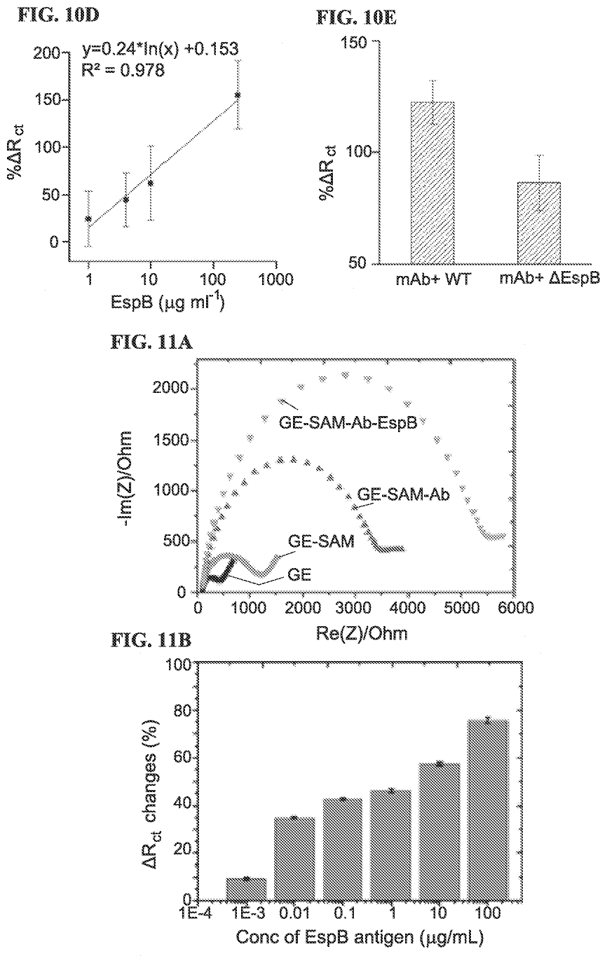

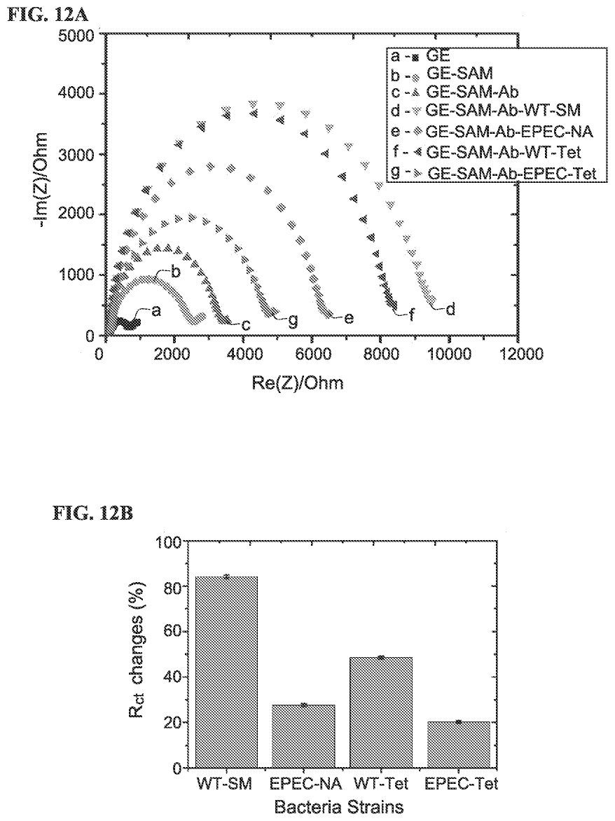

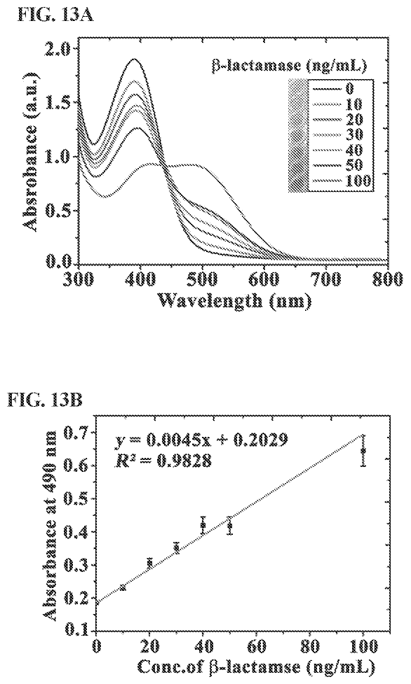

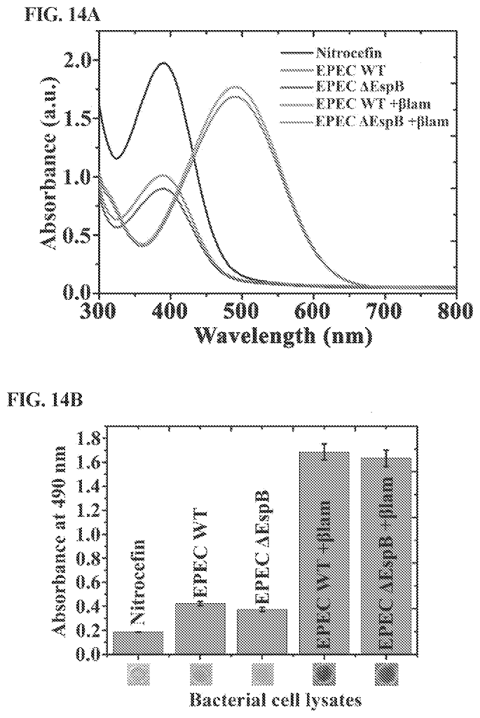

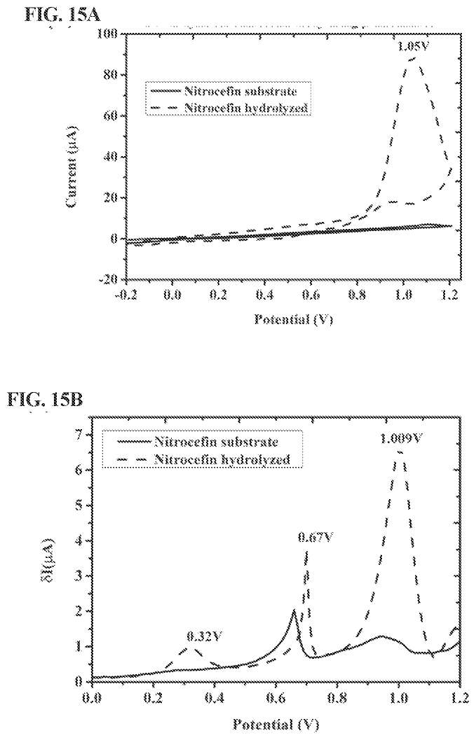

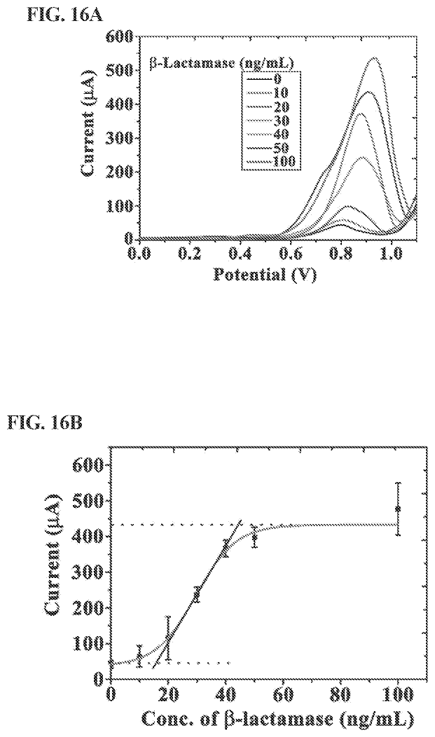

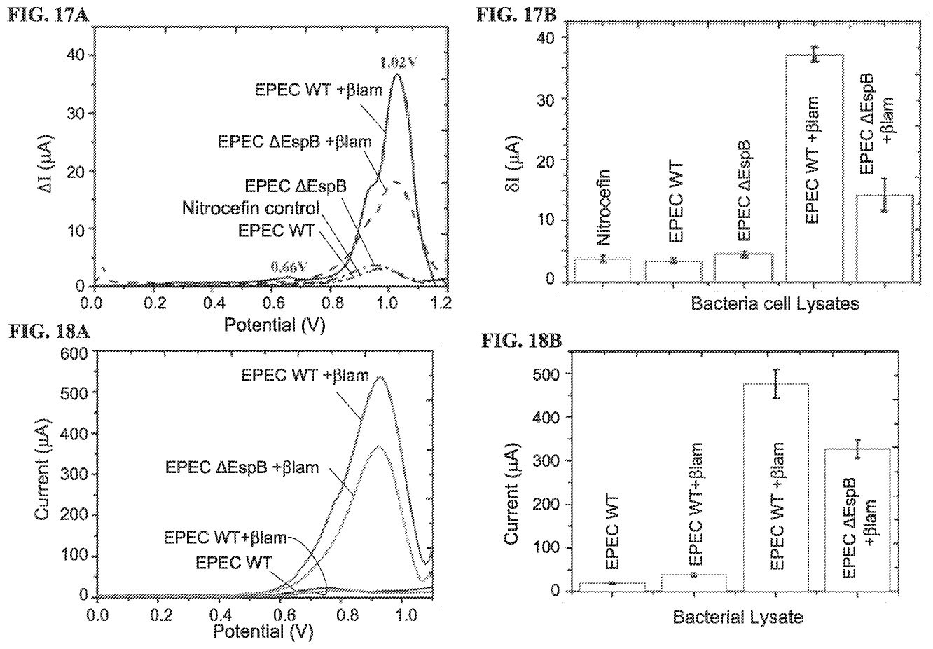

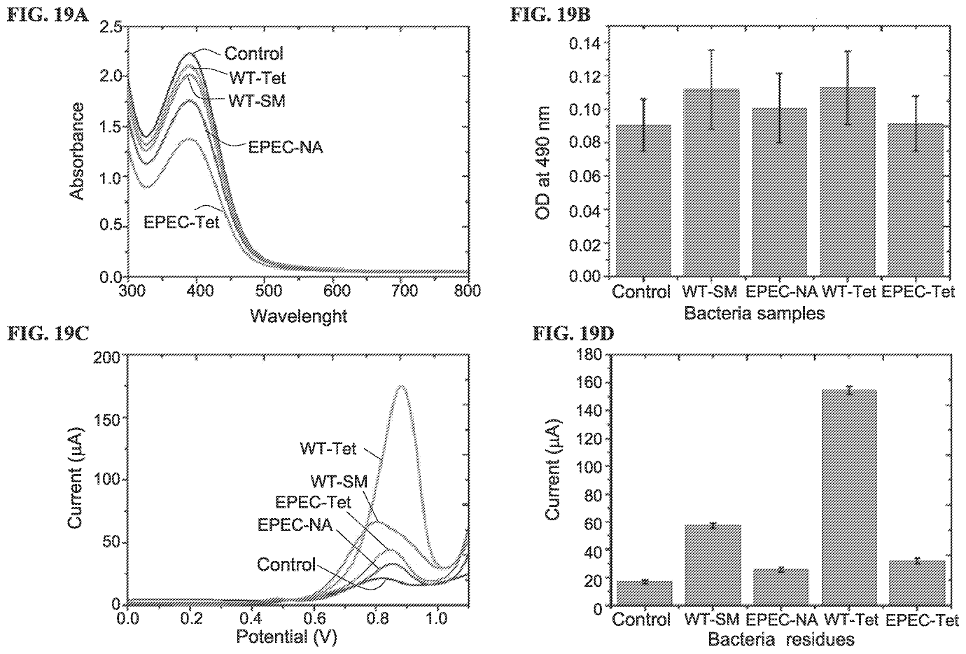

In this connection, reference is made to FIG. 4, exemplifying a specific configuration for the present methods and platform, enabling detection and profiling of antibiotic resistance of pathogens in the sample, specifically T3SS expressing bacteria. As shown, working electrode in the identification unit (channel 1) is connected either directly or indirectly to anti-EspB monoclonal antibodies selected to bind to T3SS expressing EPEC (Enteropathogenic Escherichia coli) bacteria. Presence of bound EPEC is detected by EIS providing data on presence and/or quantity of bound bacteria. The identification/quantification/capturing unit is optionally rinsed to remove excess biological material, and the targeted captured bacteria is disrupted by ultrasonic waves to cause lysis of the bound bacteria. The resulting bacteria lysates are transferred to the profiling unit (Channel 2).

The profiling unit (Channel 2) is prepared with Cephalosporin antibiotic (Nitrocefin), that is a beta-lactamase substrate connected directly or indirectly to the one or more electrodes. When interacts with β-lactamase enzymes that may exist in the bacterial lysates, the nitrocefin is hydrolyzed by β-lactamase forming an electroactive product. Detection of the hydrolyzed nitrocefin indicates that the β-lactamase enzyme/s are present in the analyzed sample, and thus provides not only diagnostic information concerning the identify and quantity of the pathogen in the sample, but also provides therapeutic useful information with respect to antibiotic resistance profile of the pathogens that exist in the sample.

In this connection, custom fabricated microelectrode chips were used for the electrochemical analysis in the profiling chamber using nitrocefin. The electrode chip contains a gold working electrode, gold counter electrode and Ag/AgCl reference electrode. In some embodiments, the electrode chip connected to voltametric circuit, Palm sense multi-channel potentiostat (MultiEmStat4) (PlamsensBV, Netherland) and accessed through PS Multi-Trace 4.4 software. Nitrocefin substrate 50 μL was loaded on to the electrode surface and cyclic voltammogram (CV) recorded with applied potential from −0.2V to +1.2V with scan rate 0.1V/s. Similarly, nitrocefin hydrolyzed by purified β-Lactamase or by bacterial cell lysates CV were also recorded, and peak currents were analyzed shown in FIG. 15A. Square wave voltammetry (SWV) measurements were carried out for all the samples with the following parameters: applied potential window: −0.2V to +1.2V; Estep: 0.01V; Amplitude: 0.01V and Frequency: 20 Hz. The nitrocefin specific currents (ΔI) were measured and analyzed as shown in FIG. 10B.

FIGS. 15A and 15B and FIGS. 17A and 17B exemplify detection of hydrolyzed nitrocefin by voltametric measurements. FIG. 15A shows cyclic voltametric measurement (CV) on nitrocefin substrate and hydrolyzed nitrocefin. FIG. 17B shows square wave voltametric (SWV) measurement on nitrocefin substrate and hydrolyzed nitrocefin. FIG. 17A shows SWV measurement of nitrocefin substrate with bacteria cell lysates from antibiotic resistant and wild type bacteria. FIG. 17B shows bar graph for measured current signal potential at 1.02V for all the bacteria cell lysates measured with nitrocefin.

The CV analysis of FIG. 15A shows no specific oxidation peak for Nitrocefin. However, following the addition of β-lactamase enzyme to nitrocefin substrate, the β-lactam ring was hydrolyzed by breaking amide bond thus increasing electronegativity. The hydrolyzed nitrocefin was filtered (using 3 kDa centrifuge filter). The filtered hydrolyzed nitrocefin product was then analyzed by CV and SWV methods. FIG. 15A CV of hydrolyzed nitrocefin shows strong irreversible oxidation peak potential at +1.05V with high current intensity (88.15 μA)—almost 15 times higher than nitrocefin substrate. This result confirms that nitrocefin has gained high electroactive property after the hydrolysis of β-lactam ring. This electroactivity is further revealed by a SWV analysis as seen in FIG. 15B. The SWV of hydrolyzed nitrocefin shows three distinct current peaks and high intensity peak (ΔI=6.5 μA) was observed at +1.009V, which was 5 times higher than the nitrocefin control. Another peak potential at +0.67 with current intensity (ΔI=4.0 μA), which was two times higher than control. A third peak was observed at lower potential at +0.32V with current intensity (ΔI=1.0 μA), which was not found in nitrocefin control. This peak originates from adsorbed specifies of hydrolyzed nitrocefin.

Similarly, as shown in FIGS. 17A to 17B, electrochemical analysis of nitrocefin assay was performed using cell lysates of β-lactamase-producing bacteria (antibiotic resistant) or antibiotic sensitive bacteria (wild-type and ΔespB).

The SWV analysis in FIG. 17A, shows two current peaks in the cell lysate of WT EPEC pCX341 NleD− βlam (Tet) with nitrocefin, the first at +1.02V demonstrating high current signal (ΔI=38 μA) and the second at +0.66V (ΔI=2 μA). Furthermore, it was found to be double than the current signal measured from EPEC ΔespB pCX341 NleD-βlam (Tet) cell lysate with nitrocefin. On the other hand, very low current signals were obtained by both wild type bacterial cell lysates with nitrocefin and nitrocefin control (ΔI=2.9-4.3 μA at +1.02V). FIG. 17B Bar graph compares the measured current signals (ΔI) at +1.02V for all bacteria cell lysates with nitrocefin and nitrocefin control. The peak currents difference in the CV and SWV results of both antibiotic resistant bacteria cell lysates is due the interference of other cellular molecules in diffusion coefficient of nitrocefin.

Generally, it should be noted that speed of voltametric measurements may affect the results. The SWV measurement were performed faster than CV analysis, possibly leading to inversion in peak heights between WT EPEC pCX341 NleD-β-lam (Tet) and EPEC ΔespB pCX341 NleD-β-lam (Tet) samples.

As shown in FIGS. 15A-15B and 17A to 17B, modified nitrocefin can be detected effectively using voltametric measurements. The use of pathogen lysates associated with a selected pathogen out of a biological sample, enables identifying that the specific pathogen in antibiotic resistant. While other, benign, bacteria in the sample may be antibiotic resistant, it may lead to over treatment and confusion. Thus, the present technique enables profiling of selected pathogen over a complete biological sample.

Still further, FIGS. 6A and 6B show characterization of the generated chips conducted using scanning electron microscopy. The characterization indicates quality of the electroplated Ag/AgCl reference electrode (RE), and of the entire cell and electrode array. The electron microscopy shows typical white luster deposit that appears, in a SEM analysis, as a homogenous crystalline deposit with dense Ag nuclei of ˜1 μm (Bar: 5 μm). Verification of a reference electrode was carried out by measuring its potential versus a commercial reference electrode in varying electrolyte (NaCl or KCl) concentrations. The RE potential demonstrated a linear dependence on the log of the electrolyte concentration, as expected, following the Nernst equation. Briefly, Verification of the newly formed Ag/AgCl reference electrode may be carried out by constructing a simple EC cell with the new RE used as indicator electrode having its potential checked versus a commercial Ag/AgCl (saturated) electrode with fixed potential. Varying concentrations of KCl solution are used in order to plot the RE response to a change in KCl concentration, according to Nernst equation. The measured potentials are plotted against log of KCl molar concentration. In theory, one should expect to obtain a potential difference of 59 mV for each log of KCl concentration. In practice, deviations from this value are expected to evolve from the nature of the measured electrode (an open reference electrode), the quality differences and experimental conditions (temperature, varying distances between the measured electrodes, which affect solution resistance, etc.).

FIGS. 7A to 7C exemplify cyclic voltammetry (CV) for a chip device as described above. FIG. 7A shows cyclic voltammetry in the presence of the electroactive redox couple ferrocyanide/ferricyanide using four different scan rates consecutively. Two measured parameters of interest on these i-E curves (cyclic voltammograms) are the ratio of peak currents, iₚₐ/i_{pc}, and the separation of peak potentials, Eₚₐ−E_{pc}. For a voltametric Nernstian wave with stable product, iₚₐ/i_{pc}=1 regardless of scan rate and diffusion coefficients. Deviation of the ratio iₚₐ/i_{pc} from unity is indicative of homogeneous kinetic or other complications in the electrode process. The peak current for a reversible process is given by the Randles-Sevick equation.

FIG. 7B shows linear peak heights increase proportional to square root of the scan rates. This agrees with the Randles-Sevick equation. FIG. 7C shows a peak separation for different scan rates, indicating that the peak separation was not significantly affected by the scan rate.

Following manufacturing of the chip device, the working electrode (e_{w}) thereof undergoes biofunctionalization to provide suitable binding sites for selected bacteria cells or other biological materials. FIGS. 8A-8B and FIGS. 9A to 9D, which are described in more details further below exemplify biofunctionalization and characterization of the working electrode according to some embodiments of the present disclosure.

FIGS. 10A to 10E illustrate the use of mAb-EspB-B7 as binding site in electrochemical chip device as described herein. FIG. 10A illustrates binding of bacterial EPEC cells to mAb-EspB-B7 and respective Nyquist plot; FIG. 10B shows Nyquist plot measurements using bare electrode, electrode carrying mAb-EspB-B7 binding sites and detection in a sample containing purified EspB protein; FIG. 10C shows relative charge transfer resistances (R_{ct}) for samples containing different amounts of charge transfer resistance compared to reference electrodes and samples; FIG. 10D show an exponential fit (using log scale) between detected R_{ct} values and EspB concentration; and FIG. 10E illustrates changes in R_{ct} for specific binding of WT EPEC cells is indicated, resulting in a larger contribution to R_{ct} compared between EPEC WT and ΔespB samples. FIG. 10A illustrates the details of EIS-based detection of whole bacterial EPEC cells. In this non-limiting example, electrochemical chips as described herein interact with bacterial EPEC cells, thereby varying impedance response along the electrode array. In this example, the electrode array includes a working electrode e_{w} radius of about 0.3 mm, counter electrode e_{c} having radius of about 0.6 mm, and a square reference electrode eᵣ having surface area of about 0.25 mm², and respective contact pads 13w,13c,13r electrically connecting thereto. The working electrode is modified with a thiolated mAb-EspB-B7 using thiol-gold chemistry. The electrodes e_{w},eᵣ,e_{c} are enclosed inside an electrochemical cell structure, configured to receive a sample. The immobilization of mAb-EspB-B7 and capture of antigen affect the impedance measured between the underlying electrodes as shown in FIGS. 10B to 10E. As shown, an EIS measurement allows for the interrogation of the electrochemical system and separation of the individual components that affect the electrochemical cell circuit established by introducing the sample into the electrochemical cell (ci). The generated Nyquist plot may be fitted to an equivalent circuit from which the different resistance values are extracted (illustrated in an inset in FIG. 10A). The Nyquist plots shown in Figured 10B were obtained by EIS measurements of a bare gold working electrode (bare GE), working electrode after the immobilization of mAb-EspB-B7 (GE+mAb) thereon, the mAb-EspB-B7-coated working electrode after incubation with 250 μg/mL purified EspB protein (GE+mAb+EspB). Variation between the Nyquist plots indicates the electrochemical effects of the binding sites and interaction thereof of materials in the sample, thus enabling characterization of the sample. A suitable one-dimensional parameter that can be extracted from the Nyquist plots, using equivalent circuit fitting, in the relative charge transfer resistance (R_{ct}) values. FIG. 10C shows measured R_{ct} values for different concentrations of purified EspB protein (1, 4, 10 and 250 μg/ml), reference sample using modified working electrode. This variation demonstrates a dose-dependent increase in the detected R_{ct} values. Relative R_{ct} values are the means of the R_{ct} ratios (before and after antigen capture) calculated from 3-6 measurements. Error bars represent the ±SD. The variation in R_{ct} was fitted to exponential formula as a function of EspB protein concentration as shown in FIG. 10D. This model provides a fit R² of 0.978 indicating good agreement with the results. FIG. 10E shows measurement of specific binding of WT EPEC cells. The specific binding is indicated by larger contribution to R_{ct} compared with the ΔespB null strain. The percent change in R_{ct} ratios measured for EPEC WT and ΔespB was calculated and averaged from 20 repeating measurements (five measurements each containing four samples) for each strain.

Thus, a first aspect of the present disclosure relates to a system comprising:
(a) a pathogen identification and/or quantification unit comprising a first electrode arrangement comprising at least one first working electrode located within at least one first chamber; and
(b) a profiling unit comprising a second electrode arrangement comprising at least one second working electrode located within at least one second chamber. The second chamber is connected to the pathogen identification unit to allow selective fluid transmission from the at least one first chamber to the at least one second chamber.

It should be noted that that the at least one first working electrode (e.g., of the pathogen identification unit of (a)) is contacted directly or indirectly to at least one target binding site and/or moiety specific for binding one or more target pathogens. Still further, the at least one second working electrode (e.g., of the profiling unit of (b)), may be in the vicinity of, in close proximity to, or may be connected directly or indirectly to at least one substrate molecule/s. It should be thus understood that in some embodiments, the at least one second electrode is attached, immobilized and/or connected directly, or indirectly via a connector or linker to the substrate. However, in some alternative or additional embodiments, at least some of the at least one second working electrodes are placed in the vicinity of a substrate, or in close proximity to at least one substrate, or in some embodiments may be placed together unconnected and/or not immobilized in at least one second chamber. Still further, it should be understood that the suitable substrate used in the present system is characterized in that the interaction of such at least one substrate molecule/s with at least one catalytic macromolecule, catalyzes the production of at least electroactive product.

In some embodiments, the substrate may be placed in the chamber, in the vicinity of the second electrode arrangement of the one or more profiling unit of the disclosed systems. “In the vicinity of” is used herein to describe physical proximity of the substrate molecule to the second electrode. In accordance with some embodiments, the term reflects the presence of the substrate in the chamber, positioned or located in close proximity to a substrate or surface, without necessarily being in direct contact with it. It should be understood that the distance between the substrate and the electrode is sufficient to allow the formation of volumetric measurements of the electroactive formed using the substrate. In other words, the substrate is placed or positioned near enough to the electrode but not necessarily in direct contact, in a manner that product formed by the catalyzed rection with the substrate will be detected by the electrode. Still further, as used herein, electroactive product is any molecule or compound that may undergo a chemical reaction and produce an electrical current or potential. More specifically, electroactive product generates an electrical signal that can be detected using an electrochemical sensor as used in the disclosed systems.

In some embodiments, the system of the present disclosure may further comprise a disruptor associated with the pathogen identification unit. Such disruptor is adapted to selectively apply disruption conditions onto one or more target pathogens bound to, or captured by, the target binding site and/or moiety in the first chamber, thereby generating pathogen lysates.

In some embodiments, the disruptor provides physical, mechanical and/or chemical conditions leading to destruction of the pathogen, while maintaining the activity of macromolecules of the pathogen.

In some embodiments, the first electrode arrangement of the disclosed system is connectable to a detection circuit adapted for electrical detection of at least one target pathogen bound to, or captured by, the at least one target binding site and/or moiety of the at least one first working electrode.

In yet some further embodiments, the detection circuit is adapted for electrochemical impedance spectroscopy (EIS), for detecting the presence and/or quantity of the at least one target pathogen bound to, or captured by the at least one target binding site and/or moiety of the at least one first working electrode of the disclosed system, specifically, of the pathogen identification unit (a).

Still further, in some embodiments, the target pathogen targeted by the disclosed system may be at least one pathogen expressing, or associated with, at least one component of the Type III Secretion System (T3SS).

In some specific embodiments, the at least one target binding site and/or moiety of the at least one first working electrode of the disclosed system, is comprised within, or may be part of, at least one antibody, or any antigen-binding fragment thereof (e.g. single-chain fragment variable¿scFv), or any immunoglobulin-like molecule that recognizes and binds at least one component of the T3SS, or any combination or complex thereof. Still further, the antibody or any functional fragments thereof (e.g., scFv) may be immobilized to, and/or connected either directly or indirectly via a linker or spacer to the at least one first working electrode of the disclosed system. It should be understood that in some embodiments, the antibody may recognize and bind at least one component of the T3SS in the context of the whole intact pathogen. Thus, a pathogen that express or is associated to at least one component of the T3SS present in the sample may be recognized and captured by the specific antibody be immobilized to the identification/quantification unit of the disclosed system. In some particular and non-limiting embodiments, the antibody applicable in the present disclosure may be a monoclonal antibody. Still further, it should be understood that various types and forms of antibodies and antigen-binding proteins applicable in the present aspect are disclosed herein after.

Still further, in some embodiments, the component of said T3SS is at least one of the EPEC secreted protein B (EspB), Enteropathogenic Escherichia coli (EPEC) secreted protein A (EspA), and EPEC secreted protein D (EspD), or any fragments or peptides thereof, and any combination or complex thereof.

In yet some further specific embodiments, the at least one antibody of the at least one first working electrode of the disclosed system recognizes and binds, and is specific for the EspB protein, even in the context of the whole intact pathogen.

In yet some further embodiments, the at least one antibody of the at least one first working electrode of the disclosed system recognizes and binds the EspB protein. More specifically, the antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 7, CDRH3 comprising the amino acid sequence ARDRRAGYFDYW, as denoted by SEQ ID NO. 8, and a light chain complementarity determining region (CDRL) 1 comprising the amino acid sequence RDNIGKNY as denoted by SEQ ID NO. 9, a CDRL2 comprising the amino acid sequence RNN as denoted by SEQ ID NO. 10, and a CDRL3 comprising the amino acid sequence SAWDTSLNA as denoted by SEQ ID NO. 11, or any derivative, variant and biosimilar thereof. It should be understood that the term antibody or derivatives thereof applicable in the disclosed systems, encompasses any type, or fragment of antibody, for example, scFv, Fab, F(ab′)2 formats, as defined in the present disclosure in connection of other aspects.

In some embodiments, the second electrode arrangement of the profiling unit of the disclosed system is connected to, or is in the vicinity of, the one or more substrate molecules such that each of the one or more second working electrode/s is in the vicinity of or is connected directly or indirectly to a single type of substrate molecule, as discussed above. The electrode arrangement is comprised within one or more second chambers.

In some embodiments, the profiling unit of the disclosed system comprises one or more second chambers. Each of the second chamber/s comprising a respective second electrode arrangement comprising one or more second working electrodes in the vicinity of, or connected directly or indirectly to one or more substrate molecules respectively. In some embodiments, each chamber may contain one type of substrate molecule. In yet some further embodiments, the profiling unit of the disclosed system may comprise multiple chambers, each containing the second electrode arrangement, and each specific for a single substrate. In some embodiments, each of the at least one second working electrodes may be either connected to the substrate molecule, or alternatively, or additionally, is in the vicinity, or is in close proximity, or comprised within the same second chamber, with the substrate molecule. Still further, in some embodiments, each of the one or more second chambers comprise a single type of substrate molecules (either connected or not connected to the second working electrode/s). According to some embodiments, the substrate molecules differ between the one or more second chambers.

In some embodiments, the interaction of the substrate molecule/s of the at least one second electrodes of the profiling unit of the disclosed system, with at least one catalytic macromolecule, leads to conversion of the substrate into at least one electroactive product. Thus, substrates useful for the profiling unit of the disclosed system are those that upon interaction thereof with catalytic macromolecules, are converted into, or form at least one electroactive product. Still further, the second electrode arrangement is connectable to a voltametric circuit adapted to provide voltametric measurement data indicative of production of the electroactive product, thereby indicating the presence of the at least one catalytic macromolecule in the pathogen lysates.

In yet some further embodiments, the catalytic macromolecule as discussed herein may be at least one enzyme. According to these embodiments, the suitable substrate molecule/s useful in the systems of the present disclosure are specific substrate/s of such enzyme. Such enzyme catalyzes the conversion of the specific substrate molecule to at least one electroactive product. In yet some further embodiments, the presence and/or association of the enzyme in a target pathogen is indicative of the pathogenicity of the target pathogen. Thus, in some embodiments, a substrate molecule provided with the profiling unit of the disclosed system allows the detection of an enzyme that causes or increases the pathogenicity of the target pathogen in a sample. This provides profiling functionally the pathogens in the sample, specifically, determining and/or evaluating the pathogenicity of the pathogens that reside in the sample.

In some specific embodiments, pathogenicity comprises antibiotic resistance. Thus, the target enzyme that may be present in the captured pathogen, may provide directly or indirectly antibiotic resistance to the target pathogen. The system, therefore, and specifically the profiling unit thereof, provide detection of enzymes that provide antibiotic resistance to the target pathogen.

In more specific embodiments, the enzyme may be at least one of: at least one hydrolase, at least one transferase, and at least one oxidoreductase.

In yet some further specific embodiments, the enzyme/s may be Lipid A phosphoethanolamine (PEtN) transferases (resistance to last resort antibiotics colistin, Aminoglycoside phosphotransferase (inactivates antibiotic amicoumacin by phosphorylation) and the like.

In some specific embodiments, the target enzyme may be at least one hydrolase. In more specific embodiments, such hydrolase may comprise at least one β-Lactamase, at least one macrolide esterase and at least one epoxide hydrolase.

Still further, in some embodiments, the enzyme is β-Lactamase. Accordingly, a suitable substrate for such β-Lactamase may be any β-lactam antibiotics or any substrate hydrolyzed by β-Lactamase to produce at least one electroactive product.

In some specific and non-limiting embodiments, a suitable substrate may be Nitorcefin. Thus, the hydrolysis of Nitorcefin, by any β-Lactamase that may be present in the sample analyzed by the disclosed systems, produces an electroactive product, thereby indicating the presence of β-Lactarnase in the target pathogen or pathogen lysates of the sample.

Additional substrates useful for the at least one second working electrode of the profiling unit of the disclosed system may include, but not limited to cephalexin, cefquinome, cefadroxil, or any other cephalosprin or otherwise a key intermediate in the synthesis of cephalosporin: 7-aminodeacetoxycephalosporanic acid (7-ADCA), CC-F2, that is a Fluorescence Resonance Energy Transfer (FRET) substrate comprising a cephalosporin core linking 7-hydroxycoumarin to fluorescein, CC-F4, CENTA β-Lactamase Substrate (a Chromogenic β-lactamase substrate) and CMPD1.

It should be understood that the platform of the present disclosure may be utilized in some embodiments to profile the catalytic activity of the identified pathogen and/or microorganism, which is not necessarily connected to its pathogenicity. The catalytic activity may be used to further characterize and monitor the identified microorganism and thus can be useful for monitoring microbial activity in industrial processes. Thus, additional embodiments for substrates that is used for or participate in, the formation of at least one electroactive product, may include nitrate that can be reduced by bacterial nitrate reductase to form nitrite that is an electroactive product that can be detected using electrochemical sensor. In yet some further embodiments, a substrate may be lactate that can be oxidized to produce pyruvate and hydrogen peroxide by the bacterial enzyme lactate oxidase. The hydrogen peroxide produced is an electroactive product. In yet some further embodiments, phenol may be converted into catechol using the enzyme phenol hydroxylase. Catechol is an electroactive product. In yet some further embodiments, cholesterol can be oxidized to cholest-4-en-3-one and hydrogen peroxide by the bacterial enzyme cholesterol oxidase. The hydrogen peroxide produced is an electroactive product.

Still further, in some embodiments, the disruptor of the disclosed system is an ultrasonic transducer configured for generating and directing an ultrasonic signal onto the at least one first chamber, thereby lysing one or more target pathogens bound to, or captured by, the target binding site and/or moiety of the detecting unit of the disclosed system.

Still further, in some embodiments, the pathogen identification unit of the disclosed system comprises at least one of:
an input port for inserting input sample; a waste port for rinsing out remaining of the input sample, thereby enabling separation of a pathogen bound to said target binding site and/or moiety; an optional filtering unit; and an output port for selectively allowing fluid transmission to the profiling unit. In some embodiments a filtering unit is used to filter out debris following bacteria disruption while filtering in any molecule with size <150 kD into the second working electrode.

Still further, in some embodiments, the system of the present disclosure further comprises a controller connectable to one or more valves and said first and second electrode arrangement; the controller is configured and operable to perform at least one of the following actions:
(a) be responsive to data indicating input of a sample to the pathogen identification unit; in response to the data, the controller operates the first electrode arrangement for detecting and/or quantifying one or more target pathogens bound to, or captured by, said target binding site and/or moiety;
(b) operation of a waste valve for rinsing out remains of said sample;
(c) operation of the disruptor to apply disruption conditions thereby generating lysates of the target pathogen;
(d) operation of a transmission valve to transmit the pathogen lysates to the profiling unit;
(e) operation of the second electrode arrangement for detecting at least one electroactive product of the one or more substrate molecules, and generation of output data indicative of catalytic activity profile of at least one catalytic macromolecule of the one or more target pathogens, in accordance with the appearance of at least one electroactive product of the substrate molecules.

Still further, in some embodiments, the disclosed systems further comprise an arrangement of one or more identification units, each comprising a first chamber and first electrode arrangement. The working electrodes of the first electrode arrangement are connected directly or indirectly to respective one or more target binding moieties, different for each of the one or more identification units. Still further, the respective one or more profiling units, each configured to receive pathogen lysates from a respective identification unit, to thereby determine catalytic activity profile of one or more target pathogens, in accordance with respective interactions of catalytic macromolecules in the pathogen lysates with one or more substrate molecules of the at least one profiling unit.

A further aspect of the present disclosure related to an array comprising plurality of systems. Each of the systems of the disclosed arrays comprise:
(a) a pathogen identification and/or quantification unit comprising a first electrode arrangement comprising at least one first working electrode located within at least one first chamber; and (b) a profiling unit comprising a second electrode arrangement comprising at least one second working electrode located within at least one second chamber. The second chamber is connected to the pathogen identification unit to allow selective fluid transmission from the at least one first chamber to the at least one second chamber. It should be further noted that the at least one first working electrode of the identification/quantification unit of the system of the disclosed array is contacted directly or indirectly to at least one target binding site and/or moiety specific for binding one or more target pathogens. Still further, the at least one second working electrode of the profiling unit of the system of the disclosed array is in the vicinity of, or is connected directly or indirectly to at least one substrate molecule/s. The interaction of the at least one substrate molecule/s with at least one catalytic macromolecule, catalyzes the production of at least electroactive product.

In some embodiments, the array of the present disclosure may comprise any of the systems disclosed by the present invention.

A further aspect of the present disclosure relates to a method for identifying and/or quantifying and/or catalytically profiling one or more target pathogens in at least one sample. The disclosed method comprises the following steps:

First in step (a), contacting the sample or any preparation thereof, with at least one first electrode arrangement comprising at least one first working electrode, or with any unit or system thereof. The at least one first working electrode may be connected directly or indirectly to at least one target binding site and/or moiety for binding and/or capturing one or more target pathogens from the sample. The next step (b), involves performing an electrochemical impedance spectroscopy (EIS) analysis of said sample; wherein impedance variations indicate the presence and/or quantity of the target pathogen in the sample. Step (c), involves applying disruption conditions to a target pathogen bound to, or captured by, the target binding site and/or moiety thereby generating target pathogen lysates. The next step (d), concerns contacting the target pathogen lysates with one or more substrate molecules, that may be in some embodiments connected directly or indirectly to the working electrodes. Alternatively or additionally, the substrate molecule/s are in the vicinity of one or more second working electrodes, of the at least one second electrode arrangement, or with any unit or system thereof. According to such embodiments, the substrate molecules may be contained in the second chamber, and once the pathogen lysates are introduced to the chamber, any catalytic macromolecule that exists in these lysates may act on the substrate present therein to produce detectable electroactive products that are detected by the at least one second electrode/s. The at least one substrate molecule suitable for the disclosed methods may be a substrate of at least one catalytic macromolecule. In case present in the lysates, the catalytic macromolecule may use the substrate for the formation of a detectable product, specifically, an electroactive product. In some embodiments, the catalytic macromolecule may catalyze the conversion of the substrate molecule to form at least one electroactive product. The next step (e), involves performing an electrochemical voltammetry or amperometry analysis of the sample to detect the production of at least one electroactive product. It should be understood that the detection of the product indicates the presence and/or activity of the catalytic macromolecule in the target pathogen lysates, thereby profiling the catalytic activity of the target pathogen in the sample.

In some alternative embodiments, the disclosed methods may further comprise rinsing remains of the biological sample away from the electrode arrangement, specifically the at least one first electrode arrangement, while keeping the pathogen attached to the target binding site and/or moiety.

In some embodiments, the methods disclosed herein may involve performing an EIS analysis of the sample, that may comprise at least one of faradic EIS and non-faradic EIS. More specifically, in some embodiments the measurement chamber may or may not contain an electroactive substrate and the measured impedimetric signal may reflect the charge transfer resistance of redox reactions of said substrate or otherwise the changes in non-faradic currents. In some embodiments, the performing an EIS analysis of the sample by the disclosed methods may comprise: (i) contacting the sample with at least one first working electrode, at least one first reference electrode, and at least one first counter electrode. The at least one first working electrode is connected directly or indirectly to at least one target binding site and/or moiety. Next (ii), measuring electrical currents between the at least one first working electrode and the at least one first reference electrode in response to alternating electric voltages at different frequencies applied between the at least one first working electrode and the at least one first counter electrode. In the next step (iii), determining electrical impedances based on the measured electrical current and the electric voltages applied at the different frequencies. Next (iv), determining a charge transfer electrical resistance based on the determined impedances; and determining presence and/or quantity of the at least one target pathogen captured to the at least one target binding site and/or moiety in accordance with the charge transfer electrical resistance.

In some embodiments, performing an electrochemical voltammetry or amperometry analysis of the sample may comprise: first (i), applying voltage signal between the at least one second working electrode and at least one reference electrode and determining electrical current through the at least one second working electrode in response to varying voltage signal. Next (ii), determining peak current value, the peak current value is inversely indicative of presence and/or quantity of the at least one electroactive product.

In some embodiments, the target pathogen identified, quantified and profiled by the methods of the present disclosure may be at least one pathogen expressing, or associated with, at least one component of the Type III Secretion System (T3SS).

In yet some further embodiments, the at least one target binding site and/or moiety of the at least one first electrode used by the disclosed methods is comprised within at least one antibody that recognizes and binds at least one component of the T3SS, or any combination or complex thereof. It should be understood that at least one component of the T3SS is recognized in some embodiments by the disclosed antibodies, in the context of the intact whole pathogen. Still further, the antibody or any functional fragments thereof is immobilized to the at least one first working electrode.

In yet some further embodiments, the component of the T3SS recognized by the antibodies used by the disclosed methods is at least one of the EspB, EspA, and EspD, or any fragments or peptides thereof, and any combination or complex thereof.

In yet some further embodiments, the at least one antibody used by the disclosed methods recognizes and binds the EspB protein.

Still further, the disclosed methods may use according to some embodiments at least one antibody recognizes and binds the EspB protein. In some specific and non-limiting embodiments, the antibody comprises a CDRH1 comprising the amino acid sequence GFTFSHYA, as denoted by SEQ ID NO. 6, CDRH2 comprising the amino acid sequence INSNGDST, as denoted by SEQ ID NO. 7, CDRH3 comprising the amino acid sequence ARDRRAGYFDYW, as denoted by SEQ ID NO. 8, and a CDRL1 comprising the amino acid sequence RDNIGKNY as denoted by SEQ ID NO. 9, a CDRL2 comprising the amino acid sequence RNN as denoted by SEQ ID NO. 10, and a CDRL3 comprising the amino acid sequence SAWDTSLNA as denoted by SEQ ID NO. 11, or any derivative, variant and biosimilar thereof.

Still further, in some embodiments, the target pathogen identified, quantified and profiled by the disclosed methods is a bacterial pathogen. In yet some further embodiments, the bacteria are Multiple Drug Resistant (MDR) bacteria.

Still further, in some embodiments, the MDR bacterium is at least one of Enteropathogenic Escherichia coli (EPEC) or Enterohemorrhagic Escherichia coli (EHEC).

In some embodiments, the disclosed methods may use any sample, specifically, the sample may be any biological sample or any environmental sample.

Still further, in some embodiments, the catalytic macromolecule that may be detected and profiled by the disclosed methods may/s be at least one enzyme. The substrate molecule used by the disclosed methods may be thus a specific substrate of the target enzyme. The enzyme catalyzes the conversion of the substrate molecule to at least one electroactive product.

A catalytic macromolecule or catalyst is a substance that causes or accelerates a chemical reaction without itself being affected. Catalysis may be classified as either homogeneous, whose components are dispersed in the same phase (usually gaseous or liquid) as the reactant, or heterogeneous, whose components are not in the same phase. Enzymes and other biocatalysts are often considered as a third category. In biocatalytic processes, natural catalysts, such as enzymes, perform chemical transformations on organic compounds.

In some embodiments, the presence and/or association of the enzyme in the target pathogen identified, quantified and profiled by the methods of the present disclosure is indicative of the pathogenicity of the target pathogen.

Still further, in some embodiments, the pathogenicity comprises antibiotic resistance. The enzyme is an enzyme that provides directly or indirectly antibiotic resistance to the target pathogen.

In some embodiments, the enzyme is at least one of: at least one hydrolase, at least one transferase, and at least one oxidoreductase.

Hydrolase, as used herein, is a class of enzyme that commonly perform as biochemical catalysts that use water to break a chemical bond, which typically results in dividing a larger molecule into smaller molecules. Some common examples of hydrolase enzymes are esterases including lipases, phosphatases, glycosidases, peptidases, and nucleosidases. Specifically, β-lactamases (beta-lactamase) are hydrolases produced by bacteria that provide multi-resistance to beta-lactam antibiotics such as penicillins, cephalosporins, cephamycins, monobactams and carbapenems (ertapenem), although carbapenems are relatively resistant to beta-lactamase. Beta-lactamase provides antibiotic resistance by breaking the antibiotics' structure. These antibiotics all have a common element in their molecular structure: a four-atom ring known as a beta-lactam (β-lactam) ring. Through hydrolysis, the enzyme lactamase breaks the β-lactam ring open, deactivating the molecule's antibacterial properties. Beta-lactam antibiotics are typically used to target a broad spectrum of gram-positive and gram-negative bacteria. Macrolide esterases are an additional example of hydrolases which provide resistance against macrolides by enzymatic cleavage of the macrolides' macrolactone ring. Macrolides belong to the polyketide class of natural products that consist of a large macrocyclic lactone ring to which one or more deoxy sugars, usually cladinose and desosamine, may be attached. The lactone rings are usually 14-, 15-, or 16-membered. A third example is epoxide hydrolases (EH's), also known as epoxide hydratases, which are enzymes that metabolize compounds that contain an epoxide residue; they convert this residue to two hydroxyl residues through an epoxide hydrolysis reaction to form diol products. Hydrolases are distinguished from each other by their substrate preferences and directly related to this, their functions.

A transferase, as used herein, may be any one of a class of enzymes that catalyze the transfer of specific functional groups (e.g. a methyl or glycosyl group) from one molecule (called the donor) to another (called the acceptor). They are involved in hundreds of different biochemical pathways throughout biology, and are integral to some of life's most important processes. An oxidoreductase, in accordance with the present disclosure, is an enzyme that catalyzes the transfer of electrons from one molecule, the reductant, also called the electron donor, to another, the oxidant, also called the electron acceptor. This group of enzymes usually utilizes NADP+ or NAD+ as cofactors. Transmembrane oxidoreductases create electron transport chains in bacteria, chloroplasts and mitochondria, including respiratory complexes I, II and III. Some others can associate with biological membranes as peripheral membrane proteins or be anchored to the membranes through a single transmembrane helix.

In yet some further embodiments, the enzyme profiled by the disclosed method may be at least one hydrolase, for example, at least one β-Lactarnase, at least one macrolide esterase and at least one epoxide hydrolase.

Still further, in some embodiments, the enzyme is β-Lactamase. In such case, the substrate that may be used by the disclosed methods may be β-lactam antibiotics or any substrate hydrolyzed by β-Lactamase to produce at least one detectable product. In some specific embodiments, at least one electroactive product.

In some embodiments, an appropriate substrate may comprise at least one beta-lactam ring. In more specific embodiments, an appropriate substrate useful in the present disclosure may be Nitorcefin. The hydrolysis of Nitorcefin produces an electroactive product, thereby indicating the presence of β-Lactamase in the target pathogen or pathogen lysates. It should be however appreciated that various substrates may be used by the disclosed method, specifically those disclosed by the present disclosure herein above.

Nitrocefin is a chromogenic cephalosporin substrate routinely used to detect the presence of beta-lactamase enzymes produced by various microbes. As a cephalosporin, nitrocefin contains a beta-lactam ring which is susceptible to beta-lactamase mediated hydrolysis. Once hydrolyzed, the degraded nitrocefin compound rapidly changes color from yellow to red. Although nitrocefin is considered a cephalosporin, it does not appear to have antimicrobial properties.

In some embodiments, the disclosed methods may be useful for the diagnosis of an infectious condition caused by or associated with at least one T3SS expressing pathogen, in a subject, and for profiling antibiotic resistance of the pathogen in the subject.

Still further, a target pathogen as used herein refers to any pathogenic agents include any pathogens, such as viruses, prokaryotic microorganisms, lower eukaryotic microorganisms, complex eukaryotic organisms, fungi, prions, parasites, yeasts, as well as toxins and venoms.

Of particular relevance are bacterial pathogens. A prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Pseudomonas sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

A lower eukaryotic organism includes yeast or fungus such as but not limited to Pneumocystis carinii, Candida albicans, Aspergillus, Histoplasma capsulatum, Blastomyces dermatitidis, Cryptococcus neoformans, Trichophyton and Microsporum.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

In some further embodiments, viral pathogen/s may be detected and/or quantified by the biosensor chip of the present disclosure. The term “viruses” is used in its broadest sense to include viruses of the families adenoviruses, papovaviruses, herpesviruses: simplex, varicella-zoster, Epstein-Barr, CMV, pox viruses: smallpox, vaccinia, hepatitis B, rhinoviruses, coronaviruses, retroviruses, zika virus, Ebola virus, hepatitis A, poliovirus, rubella virus, hepatitis C, arboviruses, rabies virus, influenza viruses A and B, measles virus, mumps virus, HIV, HTLV I and II. The term “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis. The term “parasite” includes, but not limited to, infections caused by somatic tapeworms, blood flukes, tissue roundworms, ameba, and Plasmodium, Trypanosoma, Leishmania, and Toxoplasma species.

In some embodiments, the target detected and/or quantified by the systems, arrays, methods, as well as the kits and devices derived therefrom, of the present disclosure is at least one pathogen expressing at least one component of the Type III Secretion System (T3SS). In some embodiments, the pathogen may be a bacterial pathogen expressing at least one component of the T3SS.

Still further, in some embodiments, the systems, arrays, methods, as well as the kits and devices derived therefrom of the present disclosure comprises at least one target binding site and/or moiety (in the pathogen identification unit) that may be comprised within or comprises at least one antibody that recognizes and binds at least one proteineous component of any of the disclosed pathogens. In some embodiments, the systems, arrays, methods, as well as the kits and devices derived therefrom of the present disclosure comprises at least one target binding site and/or moiety that may be comprised within at least one antibody that recognizes and binds at least one component of the T3SS, or any combination or complex thereof. In certain specific embodiments, at least one antibody is used as a target binding site, such antibody or any functional fragments thereof is directly or indirectly immobilized in some embodiments to the at least one working electrode. A wide range of Ab immobilization chemistries are applicable in the present disclosure, provided that they all must assure that the immobilized antibody strongly retained to the surface (the working electrode) in a functionally oriented fashion such that its antigen-binding sites are free to bind the antigen, that is the target discussed herein. Some include simply adsorption of the antibody onto the substrate after a prolonged incubation by passive adsorption. In yet some further embodiments, various functionalization and crosslinking strategies may be used, for example, those described by the present methods that include the direct covalent attachment of thiolated antibodies to a gold electrode surface. More specifically, the thiolation reaction is optimized to obtain an average of ˜6 —SH group per antibody by tuning the ratio of reagent to antibody. This fine-tuning enables control of the level of thiolation and ensures that antibody molecules are introduced with a sufficient number of thiols allowing their immobilization. Antibodies are thiolated in order to obtain a firm immobilization via gold-sulfur covalent bond, as discussed in the experimental procedures.

Still further, in some embodiments, the target binding site or moiety in the biosensor systems, arrays, methods, as well as the kits and devices of the present disclosure, is according to certain embodiments, at least one antibody that specifically recognizes and binds at least one component of the Type III Secretion System (T3SS) of at least one bacterium. Specifically, T3SS of Enteropathogenic Escherichia coli (EPEC).

The “Type III Secretion System or T3SS” is a complex structure composed of several subunits, which in turn are made up of approximately 20 bacterial proteins. The proteins that make up the T3SS apparatus are termed structural proteins. Additional proteins called “translocators” serve the function of translocating another set of proteins into the host cell cytoplasm. The translocated proteins are termed “effectors,” since they are the virulence factors that affect the changes in the host cells, allowing the invading pathogen to colonize, multiply, and in some cases chronically persist in the host. Briefly, the T3SS apparatus consists of two rings that provide a continuous path across the inner and outer membranes, including the peptidoglycan layer. The inner membrane ring is the larger of the two coaxial rings, and protein components that make up the inner ring have been identified for a number of bacteria. The outer membrane ring is composed of the secretin protein family, which is also known to be involved in type 2 secretion and in the assembly of type IV bacterial pili. A needle-like structure associates with the outer membrane ring and projects from the bacterial surface. It varies in length among the different pathogens and, in the case of pathogenic Escherichia coli, is extended by the addition of filaments that are thought to facilitate attachment to the host cells through the thick glycocalyx layer. Effectors are thought to be transported through the hollow tube-like needle into the host cell through the pores formed in the host cell membrane by the translocator proteins. Translocators are usually conserved among the different pathogens possessing a T3SS and show functional complementarity for secretion and translocation, whereas the effectors are most often distinct, having unique functions suited to a particular pathogen's virulence strategy. However, effector homologues also exist among different T3SS-possessing bacteria.

Still further, in some embodiments, the antibody comprised in the systems, arrays, methods, as well as the kits and devices derived therefrom of the present disclosure recognizes at least one component of the T3SS, for example, at least one of the Enteropathogenic Escherichia coli (EPEC) secreted protein A (EspA), EPEC secreted protein B (EspB), and EPEC secreted protein D (EspD), or any fragments or peptides thereof, and any combination or complex thereof.

In some embodiments, the system of the present disclosure comprises at least one antibody that recognizes and binds the EspB protein, or any fragments or peptides thereof, or any complex thereof with EspD protein.

In some embodiments, an antibody useful as a target binding site in the diagnostic biosensor systems, arrays, methods, as well as the kits and devices derived therefrom of the present disclosure, may bind the Escherichia Coli secreted protein B (EspB) expressed by the bacterium, or any fragments or peptides thereof. Thus, in some embodiments, the diagnostic biosensor systems, arrays, methods disclosed herein are used for detecting EspB expressing bacteria.

Among the virulence factors comprising the T3SS of these bacteria are the secreted proteins (Esps). The Esp responsible for the syringe-like structure of T3SS is secreted protein A (EspA), which is the needle-shaped protein of approximately 25 kDa, while secreted proteins B [Escherichia coli-secreted protein B (EspB)] and D [Escherichia coli-secreted protein D (EspD)] are responsible for the pore structure assembled in the eukaryotic membrane. Escherichia coli-secreted protein B is approximately 37 kDa in size and forms the pore assembled “needle tip” in the host cell membrane together with EspD. Also, EspB participates in phagocytosis evasion and binding to eukaryotic cell myosin, inhibition of actin interaction, and damage to the microvilli. There are three variants of EspB, i.e., α, β, and γ, where the α variant is subdivided into 1, 2, and 3. Allele frequency studies have shown a EspB to be the most prevalent, followed by β EspB.

In some embodiment, the EspB protein comprises the amino acid sequence as denoted by SEQ ID NO: 1 (Accession number: WP_001091991.1), or any homologs or derivatives thereof. In some specific embodiments, the EspB protein is encoded by a nucleic sequence as denoted by SEQ ID NO: 2 (Accession number: AAB69980.1), or any homologs or derivatives thereof.

In some further embodiments, the EspD protein comprises the amino acid sequence as denoted by SEQ ID NO: 3 (Accession number: WP_000935767.1), or any homologs or derivatives thereof. In some specific embodiments, the EspD protein is encoded by a nucleic sequence as denoted by SEQ ID NO: 4 (Accession number: CA143861.1). In some further embodiments, the EspA protein comprises the amino acid sequence as denoted by SEQ ID NO: 10 (Accession number: UniProtKB—Q47184 (Q47184_ECOLX)), or any homologs or derivatives thereof.

In some embodiments, the isolated antibody used in the diagnostic biosensor systems, kits and methods of the invention, specifically recognizes and binds an epitope comprising residues 185 to 250, specifically residues 190 to 215, more specifically, residues 193 to 210 of the EspB protein, specifically, the EspB protein that comprises the amino acid sequence as denoted by SEQ ID NO. 1. In yet some further embodiments, the epitope recognized by the antibody used as the target binding site and/or moiety in the pathogen identification unit of the present disclosure may comprise the amino acid sequence of TSAQKASQVAEEAADAAQ, or at least part thereof. In yet some further embodiments, the epitope recognized by the antibody disclosed herein may comprise the amino acid sequence as denoted by SEQ ID NO: 5.

In some specific embodiments, the systems, arrays, methods, as well as the kits and devices derived therefrom of the present disclosure comprises (optionally directly or indirectly immobilized therein) at least one antibody that recognizes and binds the EspB protein. In more specific and non-limiting embodiments, the antibody of the system of the present disclosure comprises a heavy chain complementarity determining region (CDRH) 1 comprising the amino acid sequence GFTFSHYA, as denoted by SEQ ID NO: 6, CDRH2 comprising the amino acid sequence INSNGDST, as denoted by SEQ ID NO: 7, CDRH3 comprising the amino acid sequence ARDRRAGYFDYW, as denoted by SEQ ID NO: 8, and a light chain complementarity determining region (CDRL) 1 comprising the amino acid sequence RDNIGKNY as denoted by SEQ ID NO: 9, a CDRL2 comprising the amino acid sequence RNN (Arg, Asn, Asn), and a CDRL3 comprising the amino acid sequence SAWDTSLNA as denoted by SEQ ID NO: 11, or any derivative, variant and biosimilar thereof. As used herein, the term “biosimilar” relates in some embodiments, to a biological product, for example, proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97 percent or greater to the amino acid sequence of its reference medicinal product, e.g., 97 percent, 98 percent, 99 percent or 100 percent. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product.

In some embodiments, the antibody may comprise a heavy chain complementarity determining region (CDRH) 1 comprising the amino acid sequence GFTFSHYA, as denoted by SEQ ID NO. 6, or any homologs or derivatives thereof, CDRH2 comprising the amino acid sequence INSNGDST, as denoted by SEQ ID NO. 7, or any homologs or derivatives thereof, CDRH3 comprising the amino acid sequence ARDRRAGYFDYW, as denoted by SEQ ID NO. 8, or any homologs or derivatives thereof, and a light chain complementarity determining region (CDRL) 1 comprising the amino acid sequence RDNIGKNY as denoted by SEQ ID NO. 9, or any homologs or derivatives thereof, a CDRL2 comprising the amino acid sequence RNN (Arg, Asn, Asn), or any homologs or derivatives thereof, and a CDRL3 comprising the amino acid sequence SAWDTSLNA as denoted by SEQ ID NO. 11, or any homologs or derivatives thereof, or any derivative, variant and biosimilar of the antibody of the present disclosure.

In some embodiments, the antibody may comprise a heavy chain variable region and a light chain variable region, specifically, comprising CDR sequences as described above. In some specific embodiments, the heavy chain variable region is encoded by a nucleic acid sequence which may be at least 70% identical to the nucleic acid sequence denoted by SEQ ID NO.12, or any homologs or derivatives thereof. In yet some further embodiments, the light chain variable region is encoded by a nucleic acid sequence which is at least 70% identical to SEQ ID NO.14, or any homologs or derivatives thereof. Still further, in some embodiments, about 75%, 80%, 85%, 90%, 95%, 99% or 100% identity.

In some other embodiments, the antibody may comprise a heavy chain variable region comprising the amino acid sequence denoted by SEQ ID NO.13 or any homologs, derivatives or variants thereof and a light chain variable region comprising the amino acid sequence denoted by SEQ ID NO.15 or any homologs, derivatives or variants thereof.

In more specific embodiments, the isolated monoclonal antibody or any antigen-binding fragment thereof may comprise a Heavy chain Framework Region 1 (FR1) comprising the amino acid sequence denoted by SEQ ID NO: 16, or any homologs or derivatives thereof, a heavy chain FR2 comprising the amino acid sequence denoted by SEQ ID NO: 17, or any homologs or derivatives thereof and a heavy chain FR3 comprising the amino acid sequence denoted by SEQ ID NO: 18, or any homologs or derivatives thereof, and a Light chain Framework Region 1 (FR1) comprising the amino acid sequence denoted by SEQ ID NO: 19, or any homologs or derivatives thereof, a Light chain FR2 comprising the amino acid sequence denoted by SEQ ID NO: 20, or any homologs or derivatives thereof, and a Light chain FR3 comprising the amino acid sequence denoted by SEQ ID NO: 21, or any homologs or derivatives thereof.

As indicated above, the target binding site used the systems, arrays, methods, as well as the kits and devices of the present disclosure, may comprise an antibody or any fragments thereof. The term “antibody” as used herein, means any antigen-binding molecule or molecular complex that specifically binds to or interacts with a particular antigen of any fragments thereof. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region (CH). The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Typically, an antibody is composed of two immunoglobulin (Ig) heavy chains and two Ig light chains. In humans, antibodies are encoded by three independent gene loci, namely kappa (κ) chain (Igκ) and lambda (λ) chain (Igλ) genes for the Light chains and IgH genes for the Heavy chains, which are located on chromosome 2, chromosome 22, and chromosome 14, respectively.

The antibody of the invention may be a monoclonal antibody, and in some embodiments a humanized or human antibody or any antigen-binding fragment thereof. In some embodiments, the antibody of the invention is a monoclonal antibody. A monoclonal antibody, as used herein refers to an antibody produced by a single clone of cells or cell line producing identical antibody molecules. Monoclonal antibodies display monovalent affinity in binding the same epitope. It should be further understood that the present invention further encompasses any functional fragments of then antibody of the invention, such fragments are referred to herein as antigen binding fragments. The term “an antigen-binding fragment” refers to any portion of an antibody that retains binding to the antigen.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), camelid antibodies, and shark variable IgNAR domains, are also encompassed within the expression “antigen-binding fragment,” as used herein.

Examples of antibody functional fragments include but are not limited to a single-domain antibody (sdAb) which refers to an antibody fragment consisting of a single monomeric variable antibody domain. The first single-domain antibodies were engineered from heavy-chain antibodies found in camelids; these are called VHH fragments. Cartilaginous fishes also have heavy-chain antibodies (IgNAR, ‘immunoglobulin new antigen receptor’), from which single-domain antibodies called variable new antigen receptor antibody (V-NAR) fragments can be obtained.

An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes. Thus, it should be further appreciated that in some embodiments, the invention further encompasses a polypeptide comprising a variable region of a light chain comprising at least one of the CDR comprising the amino acid sequences as denoted by SEQ ID NO: 9 (CDRL1), RNN (CDRL2) and SEQ ID NO:11 (CDRL3), or any homologs or derivatives thereof. In yet some further embodiments, the polypeptide of the invention may comprise the sequence of a variable region, as denoted by SEQ ID NO: 15, or any homologs thereof. In yet some further embodiments, the present disclosure further provides a polypeptide comprising a variable region of an antibody heavy chain. In some specific embodiments, such polypeptide may comprise the amino acid sequence of at least one of the following CDRs, specifically, CDRs comprising the amino acid sequences as denoted by any one of SEQ ID NO: 6 (CDRH1), SEQ ID NO: 7 (CDRH2) and SEQ ID NO: 8 (CDRH3), or any homologs or derivatives thereof. In yet some further embodiments, the polypeptide of the invention may comprise the variable region of the heavy chain as denoted by SEQ ID NO: 13, or any homologs or derivatives thereof.

As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes multivalent antibodies, specifically, bivalent molecules, diabodies, triabodies, tetrabodies and the like.

References to “V_{H}” or a “VH” refer to the variable region of an immunoglobulin heavy chain, including an Fv, scFv, a disulfilde-stabilized Fv (dsFv) or Fab. References to “V_{L}” or a “VL” refer to the variable region of an immunoglobulin light chain, including of an Fv, scFv, dsFv or Fab.

More specifically, the phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain antibody applicable for the invention, e.g., may bind as a monomer. Other exemplary single chain antibodies may form diabodies, triabodies, and tetrabodies.

It should be appreciated that in some embodiments, any antibody provided by the present disclosure and used by the diagnostic biosensor system, arrays methods, and kits and devices of the present disclosure is not a naturally occurring antibody. Specifically, any of the antibodies used herein cannot be considered as a product of nature. In yet some further embodiments, it should be noted that the epitope recognized by the antibodies of the invention may comprise, at least part of residues 185 to 250, specifically, residues 190 to 215, more specifically, 193 to 210 of the EspB protein, specifically, the EspB as denoted by SEQ ID NO. 1. Still further, in some embodiments, the antibody of the invention comprises at least part of the amino acid sequence TSAQKASQVAEEAADAAQ, as denoted by SEQ ID NO: 5. According to Donnenberg et al. (Donnenberg et a. (2011) Journal of Bacteriology; p 2972-2980), the EspB protein adopts a transmembrane topology with its C-terminus facing the host cytoplasm. Therefore, the epitope should be found inside the host cell following bacterial infection. It should be appreciated that the invention further encompasses in some embodiments thereof any antibody that recognizes and binds an epitope comprising the amino acid sequence as denoted by SEQ ID NO: 5, or any homologs or derivatives thereof.

The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three-dimensional structural characteristics as well as specific charge characteristics.

In yet some further embodiments, the antibody of the present disclosure cannot be considered as naturally occurring antibody. As such, the antibody of the invention is not a product of nature. Still further, it should be understood, that in some embodiments thereof, the invention further encompasses the use of any antibody that competes with any of the antibodies disclosed herein, specifically, any antibody that competes with an antibody comprising at least one of the CDRs as denoted by SEQ ID NO: 6 (CDRH1), SEQ ID NO: 7 (CDRH2) and SEQ ID NO: 8 (CDRH3), SEQ ID NO: 9 (CDRL1), RNN (CDRL2) and SEQ ID NO:11 (CDRL3), or any homologs or derivatives thereof. In yet some further embodiments, the invention further encompasses any antibody that competes with an antibody comprising the variable heavy chain as denoted by SEQ ID NO: 13, or any homologs or derivatives thereof, and/or the variable light chain that comprises the amino acid sequence as denoted by SEQ ID NO: 15, or any homologs or derivatives thereof. In yet some further embodiments, the term “competes” as used herein refers to any competition that results in reduction, attenuation, decrease or inhibition of binding of at least one of, the binding of the antibody of the invention to its epitope.

The invention relates to the use of antibodies that are polypeptides comprising amino acid sequences. “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

It should be appreciated that the invention encompasses the use of any variant or derivative of the antibody of the invention and any antibodies that are substantially identical or homologue to the antibodies encoded by the nucleic acid sequence of the invention. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, specifically antibodies described herein, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention, for example, any of the antibodies that comprise the amino acid sequence as denoted by any one of SEQ ID NO. 13 and 15, or any one of the CDRs of SEQ ID NO: 6 (CDRH1), SEQ ID NO: 7 (CDRH2) and SEQ ID NO: 8 (CDRH3), SEQ ID NO: 9 (CDRL1), RNN (CDRL2) and SEQ ID NO:11 (CDRL3). Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 2 and 18 specifically, the entire sequence as denoted by SEQ ID NO: 13 and 15, or any one of the CDRs of SEQ ID NO: 6 (CDRH1), SEQ ID NO: 7 (CDRH2) and SEQ ID NO: 8 (CDRH3), SEQ ID NO: 9 (CDRL1), RNN (CDRL2) and SEQ ID NO:11 (CDRL3).

In some embodiments, derivatives refer to antibodies, which differ from the antibodies specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the antibodies of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the antibodies of the preset disclosure.

It should be appreciated that any of the disclosed antibody variants must retain the ability of specific recognition and binding of any target pathogen expressing or associates with the EspB protein or any fragments thereof, or any other TSS3 component.

In some embodiments, the disclosure relates to a biosimilar derived from the mAb-B7 antibody described above.

In some embodiments, the systems, arrays, methods, as well as the kits and devices derived therefrom, of the present disclosure is usable for detecting the presence of a pathogen expressing at least one T3SS component in a sample. In some embodiments, such a pathogen is a bacterial pathogen. In yet some further embodiments, the at least one bacteria is at least one Multiple Drug Resistant (MDR) bacteria.

In more specific embodiments, the MDR bacteria is at least one of Enteropathogenic Escherichia coli (EPEC) and Enterohemorrhagic Escherichia coli (EHEC).

In some embodiments, a sample that may be used for the systems, arrays, methods, as well as the kits and devices derived therefrom, of the present disclosure may be a biological sample or an environmental sample, as will be described herein after.

It should be noted that the term “bacterium” or “bacteria” as used herein refers to any of the prokaryotic microorganisms that exist as a single cell or in a cluster or aggregate of single cells. In more specific embodiments, the term “bacteria” specifically refers to Gram negative bacteria, or a Gram-positive bacteria, specifically, a Gram negative bacteria. In some embodiments, the at least one bacterium referred herein may be a gram-negative bacteria.

The present disclosure therefore provides diagnostic biosensor systems, kits and methods for detecting T3SS expressing bacteria in a sample. It should be noted that the term “bacterium” or “bacteria” as used herein refers to any of the prokaryotic microorganisms that exist as a single cell or in a cluster or aggregate of single cells. In more specific embodiments, the term “bacteria” specifically refers to Gram negative bacteria, or a Gram-positive bacteria, specifically, a Gram negative bacteria. In some embodiments, the at least one bacterium referred herein may be a gram-negative bacteria.

While the Gram-positive bacteria are recognized as retaining the crystal violet stain used in the Gram staining method of bacterial differentiation, and appear to be purple-colored under a microscope, the Gram-negative bacteria do not retain the crystal violet, making positive identification possible. In other words, the term bacteria apply herein to bacteria with a thin peptidoglycan layer of their cell wall that is sandwiched between an inner cytoplasmic cell membrane and a bacterial outer membrane (Gram-negative).

In yet some other embodiments, the bacteria relevant to the antibody of the invention may be at least one Multiple Drug Resistant (MDR) bacteria.

As used herein, the term “resistance” is not meant to imply that the bacterial cell population is 100% resistant to a specific antibiotic compound, but includes bacteria that are tolerant of the antibiotics or any derivative thereof. More specifically, the term “bacterial resistance gene/s” refers to gene/s conferring about 100%, 90%, 80%, 70%, 60%, 50%, 40%, 30%, 20%, or 10% protection from an antibiotic compound, thereby reversing susceptibility and sensitivity thereof to said antibiotic compound.

Bacteria of particular interest may be any bacteria involved in nosocomial infections or any mixture of such bacteria. The term “Nosocomial Infections” refers to Hospital-acquired infections, namely, an infection whose development is favored by a hospital environment, such as surfaces and/or medical personnel, and is acquired by a patient during hospitalization. Nosocomial infections are infections that are potentially caused by organisms resistant to antibiotics. Nosocomial infections have an impact on morbidity and mortality, and pose a significant economic burden. In view of the rising levels of antibiotic resistance and the increasing severity of illness of hospital in-patients, this problem needs an urgent solution. Common nosocomial organisms include Clostridium difficile, methicillin-resistant Staphylococcus aureus, coagulase-negative Staphylococci, vancomycin-resistant Enteroccocci, resistant Enterobacteriaceae, Pseudomonas aeruginosa, Acinetobacter and Stenotrophomonas maltophilia.

The nosocomial-infection pathogens may be Gram-negative rod-shaped organisms (Klebsiella pneumonia, Klebsiella oxytoca, Escherichia coli, Proteus aeruginosa, Serratia spp.), Gram-negative bacilli (Enterobacter aerogenes, Enterobacter cloacae), aerobic Gram-negative coccobacilli (Acinetobacter baumanii, Stenotrophomonas maltophilia) and Gram-negative aerobic bacillus (Stenotrophomonas maltophilia, previously known as Pseudomonas maltophilia). Among many others Pseudomonas aeruginosa is an extremely important nosocomial Gram-negative aerobic rod pathogen.

“ESKAPE” pathogens may also be of particular interest. As indicated herein, these pathogens include but are not limited to Enterococcus faecium, Staphylococcus aureus, Klebsiella pneumoniae, Acinetobacter baumanii, Pseudomonas aeruginosa, and Enterobacter.

In further embodiments, the bacteria as referred to herein by the invention may include Yersinia enterocolitica, Yersinia pseudotuberculosis, Salmonella typhi, Pseudomonas aeruginosa, Vibrio cholerae, Shigella sonnei, Bordetella Pertussis, Plasmodium falciparum, Chlamydia trachomatis, Bacillus anthracis, Helicobacter pylori and Listeria monocytogens.

In some other specific embodiments, the bacteria referred to herein may be a gram negative. In other specific embodiments, the target cells of interest may be any E. coli strain, specifically, any one of 0157:H7, enteroaggregative (EAEC), enterohemorrhagic (EHEC), enteroinvasive (EIEC), enteropathogenic (EPEC), enterotoxigenic (ETEC) and diffuse adherent (DAEC) E. coli.

In some further embodiments, the MDR bacteria detected by the diagnostic systems, kits and methods of the present disclosure may be least one of Enteropathogenic Escherichia coli (EPEC) and Enterohemorrhagic Escherichia coli (EHEC). Enteropathogenic Escherichia coli and EHEC are the main bacterial agents associated with diarrhea among children under 5 years old, and both pathogens are able to induce the A/E lesion.

In some embodiments, the MDR bacteria may be Enteropathogenic Escherichia coli (EPEC).

In some other embodiments, the MDR bacteria may be C. rodentium.

In more specific embodiments, the MDR bacteria is at least one of Enteropathogenic Escherichia coli (EPEC) and Enterohemorrhagic Escherichia coli (EHEC).

In some further embodiments, the antibody of the systems, kits and methods of the present disclosure recognizes and binds at least one component of the T3SS of at least one MDR bacteria, and therefore provides the diagnosis of an MDR bacteria in a sample, or in a subject. In some further embodiments, the MDR bacteria may be at least one of EPEC and EHEC.

Specifically concerning the EPEC and EHEC bacteria, the hallmark of EPEC and EHEC-induced intestinal pathology is the attaching and effacing (A/E) lesion, whose formation depends on a T3SS encoded within the loci of enterocyte effacement (LEE) and the interplay of many T3SS effectors. Following intimate attachment of the bacteria to the intestinal epithelium, the brush border microvilli are disrupted (effacement), and the bacteria promote formation of actin pedestals that elevate the pathogen above the intestinal epithelium. To attach to the enterocytes, EPEC and EHEC utilize their T3SSs to inject the Translocated Intimin Receptor (Tir) into the host cell, where it inserts into the host cell membrane and binds to the bacterial outer membrane protein intimin. Binding of intimin to Tir induces Tir clustering, initiating a cascade of signaling events that leads to actin polymerization and pedestal formation. This ultimately results in the formation of the A/E lesion. EPEC Tir is tyrosine phosphorylated to recruit the Arp2/3 complex and drive actin polymerization, whereas EHEC Tir is not phosphorylated but, rather, relies on an additional T3SS effector, TccP/EspFU, for Arp2/3 recruitment. Successful pedestal formation requires downregulation of filopodia, which form in response to EPEC/EHEC infection, as well as disruption of the host microtubule network. The T3SS effectors Map (mitochondrion-associated protein), Tir, EspH (153), EspG, and EspG2 mediate these processes. This multifaceted approach allows A/E pathogens to coordinate the formation of A/E lesions and actin pedestals, providing them with a unique niche in the intestine of the infected host.

In some more specific embodiments, the T3SS recognized by the antibody used by the methods of the invention may be an MDR bacteria, in some specific embodiments, such bacteria may be Enteropathogenic Escherichia coli (EPEC).

In yet another embodiment, the bacteria may induce attaching and effacing (A/E) lesion in the subject.

In some further embodiments, the bacteria referred to herein may be C. rodentium.

In some further embodiments, the antibody used in the biosensor system, kits and methods of the invention may recognize the EspB expressed by the bacteria, as specified above.

In some embodiments, the methods of the invention may use, and thus may be applicable for identifying and/or quantifying of a target in a biological sample or an environmental sample.

The terms “sample”, “test sample” and “specimen”, “biological sample” are used interchangeably in the present specification and claims and are used in its broadest sense. They are meant to include both biological and environmental samples and may include an exemplar of synthetic origin. This term refers to any media that may contain the T3SS expressing bacteria and may include body fluids (urine, blood, milk, cerebrospinal fluid, rinse fluid obtained from wash of body cavities, phlegm, pus), samples taken from various body regions (throat, vagina, ear, eye, skin, sores), food products (both solids and fluids) and swabs taken from medicinal instruments, apparatus, materials), as well as substances in which controlled chemical reactions are being carried out. More specifically, according to certain embodiments, the method of the invention uses any appropriate biological sample. The term “biological sample” in the present specification and claims is meant to include samples obtained from any subject or environmental sources, for example, a mammal subject. It should be recognized that in certain embodiments a biological sample may be for example, blood cells, blood, serum, plasma, bone marrow, lymph fluid, urine, sputum, saliva, feces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents.

In certain embodiment, the biological sample suitable for the method of the present disclosure may be any one of serum, whole blood sample, urine, saliva, or any fraction or preparation thereof.

In some embodiments, the sample applicable in the biosensor systems, arrays, methods, as well as the kits and devices derived therefrom, in accordance with the present disclosure may be either as naturally obtained from the tested subject or manipulated and prepared. In some embodiments, the body fluid samples may be concentrated samples. In yet some further embodiments, the serum samples may be diluted and as such, different sera concentrations may be used. In some further embodiments the serum concentration may range between about 0.01% and 100%, More specifically, 0.01%, 0.02%, 0.03%, 0.04%, 0.05%, 0.06%, 0.07%, 0.08%, 0.09%, 0.1%, 0.2%, 0.2%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20%, 25%, 30%, 35%, 40%, 45%, 50%, 55%, 60%, 65%, 70%, 75%, 80%, 85, 90%, 95%, 100% or more. In more specific embodiment, the sample concentration may range between about 1% to about 20%, in yet some further particular embodiments, the sample concentration of the sample may be 5%.

It should be further noted that in some embodiments, the diagnostic biosensor systems, arrays, methods, as well as the kits and devices derived therefrom, in accordance with the present disclosure may be also applicable for environmental samples. Environmental samples include environmental material such as surface matter, earth, soil, water, air and industrial samples, as well as samples obtained from food and dairy processing instruments, apparatus, equipment, utensils, disposable and non-disposable items. These examples are not to be construed as limiting the sample types applicable to the present invention. The sample may be any media, specifically, a liquid media that may contain the T3SS expressing bacteria. Typically, substances and samples or specimens that are a priori not liquid may be contacted with a liquid media which is contacted with the biosensor system of the invention.

More specifically, by the term “food”, it is referred to any substance consumed, usually of plant or animal origin. Some non-limiting examples of animals used for feeding are cows, pigs, poultry, etc. The term food also comprises products derived from animals, such as, but not limited to, milk and food products derived from milk, eggs, meat, etc.

In some specific embodiments, the present invention encompasses samples of a substance, which is used as a drink. A drink or beverage is a liquid which is specifically prepared for human consumption. Non limiting examples of drinks include, but are not limited to water, milk, alcoholic and non-alcoholic beverages, soft drinks, fruit extracts, etc.

In yet some further embodiments, the method of the present disclosure, that detects, identify and/or quantify and/or profile at least one pathogen in a sample, is used for the diagnosis of an infectious condition caused by or associated with at least one T3SS expressing pathogen, in a subject. According to these embodiments, the sample used by the disclosed method is at least one sample of the subject.

The present disclosure therefore provides a powerful diagnostic and therapeutic tool for rapid diagnosis of patients suffering from infectious condition caused by, or associated with, at least one bacteria expressing at least one T3SS, together with profiling the drug resistance of these pathogens.

A further aspect of the present disclosure relates to a diagnostic method. More specifically, provided herein is a method for diagnosing an infectious disease caused by at least one pathogen in a subject. The method comprises the step of detecting, identifying, quantifying and/or catalytically profiling one or more target pathogens in at least one sample of the subject. More specifically, the method comprising the following steps: In step (a), contacting the sample with at least one first electrode arrangement comprising at least one first working electrode, or with any unit or system thereof. The at least one first working electrode is connected directly or indirectly to at least one target binding site and/or moiety for binding and/or capturing one or more target pathogens present in the sample. Step (b) of the disclosed methods involves performing an electrochemical impedance spectroscopy (EIS) analysis of the sample. It should be understood that impedance variations, indicate the presence and/or quantity of the target pathogen in said sample. Step (c) involves applying disruption conditions to a target pathogen bound to, or captured by, the target binding site and/or moiety thereby generating target pathogen lysates. Next, in step (d), contacting the target pathogen lysates with one or more substrate molecules connected directly or indirectly to, or in the vicinity of, one or more second working electrodes of at least one second electrode arrangement, or with any unit or system thereof. It should be noted that the at least one substrate molecule is a substrate of at least one catalytic macromolecule that may be found in the sample. More specifically, the catalytic macromolecule catalyzes the formation of at least one electroactive product using the substrate. In some embodiments, the catalytic macromolecule may catalyze the conversion of the substrate to form at least one electroactive product. Step (e), involves performing an electrochemical voltammetry or amperometry analysis of the sample to detect the production of at least one electroactive product. It should be understood that the detection of the product indicates the presence and/or activity of the catalytic macromolecule in the target pathogen lysates.

The disclosed method thereby provides diagnosis of an infectious disease caused by at least one pathogen in the subject, identification and/or quantification of the pathogen and/or profiling the catalytic activity of the pathogen in the subject.

In some embodiments of the disclosed diagnostic methods, the step of detecting, identifying, quantifying and/or catalytically profiling one or more target pathogens in at least sample of the subject may be performed by a method as defined by the present disclosure herein above.

Still further, in some embodiments, the pathogen causing the infectious disease diagnosed by the disclosed diagnostic methods, may be at least one T3SS expressing pathogen. The disclosed methods further provide profiling the catalytic activity of the pathogen. More specifically, such catalytic activity may comprise and reflect profiling antibiotic resistance of the T3SS pathogen in the subject. Thus, in some specific embodiments, the disclosed diagnostic method may be particularly applicable for the diagnosis of an infectious condition caused by or associated with at least one T3SS expressing pathogen.

The disclosed diagnostic methods provide a powerful therapeutic tool. Thus, in accordance with a further aspect of the present disclosure relates to a method of treating, preventing, ameliorating, reducing or delaying the onset of an infection by at least one bacteria expressing at least one T3SS in a subject in need thereof. The disclosed therapeutic methods involve the diagnostic step as discussed above, together with profiling any pathogen that may exist in the sample, by providing valuable information with respect to the presence and/or activity of catalytic macromolecules that may be expressed by or associated with the pathogen in the sample. This information enables the evaluation of the pathogenicity of the pathogen and provides effective treatment regimen. Thus, in some embodiments, the therapeutic methods provided herein comprise:

First (a), classifying a subject as a subject infected by a bacterial pathogen if the presence of at least one T3SS component is determined in at least one sample of the subject. The second step (b), involves determining the antibiotic resistance profile of the bacterial pathogen in a sample of the subject. The determination of the presence of the at least one T3SS component in the sample, and profiling the antibiotic resistance of the bacteria is performed by the steps of: First (i), contacting at least one sample of the subject with at least one first electrode arrangement comprising at least one first working electrode, or with any unit or system thereof. The at least one first working electrode is connected directly or indirectly to at least one target binding site and/or moiety for binding and/or capturing one or more target pathogens from the sample. Next (ii), performing an EIS analysis of the sample. It should be noted that impedance variations indicate the presence and/or quantity of the target pathogen in the sample. The next step (iii), involves applying disruption conditions to a target pathogen bound to, or captured by, the target binding site and/or moiety thereby generating target pathogen lysates. The resulting target pathogen lysates in the next step (iv), are contacted with one or more substrate molecules connected directly or indirectly to one or more second working electrodes of at least one second electrode arrangement, or with any unit or system thereof. The at least one substrate molecule is a substrate of at least one enzyme providing antibiotic resistance to the pathogen. Still further, the enzyme catalyzes the conversion of the substrate molecule to form at least one electroactive product. The next step (v) involves performing an electrochemical voltammetry or amperometry analysis of the sample to detect the production of at least one electroactive product. The detection of the product indicates the presence and/or activity of the enzyme in the target pathogen lysates, thereby profiling the antibiotic resistance of the target pathogen in the sample. The next step (c), of the therapeutic methods involves administering to a subject classified as an infected subject in step (a), a therapeutically effective amount of at least one anti-bacterial agent, in accordance with the antibiotic resistance profile determined in step (b).

In some embodiments, the determination of the presence of the at least one T3SS component in the sample and profiling of the pathogen is performed by the methods as defined by the present disclosure.

The clinical spectrum of disease caused by T3SS-containing pathogens is remarkably broad. Infection with enteropathogenic and enterohemorrhagic E. coli (EPEC and EHEC, respectively), Shigella, Salmonella, and Yersinia species results in intestinal disease. Yersinia pestis is the causative agent of plague. Salmonella serovar typhi causes enteric fever. Bordetella causes whooping cough, while the opportunistic pathogen Pseudomonas aeruginosa can cause a variety of problems, including pneumonia, urinary tract infection, wound infection, septicemia, and endocarditis. Chlamydia trachomatis is a common sexually transmitted organism, and Chlamydia pneumoniae causes pneumonia and has been implicated in atherosclerotic disease of blood vessels. Burkholderia pseudomallei causes community-acquired bacteremia and pneumonia. Whether by a direct toxic mechanism or through induction of self-damaging host responses, the virulence of all of these bacteria utilizes T3SSs. Clearly, T3SSs are not restricted to a specific pathogen, tissue, host environment, clinical disease spectrum, or patient population.

In some further embodiments, the diagnostic and therapeutic methods of the present disclosure may be applicable for diagnosing any infection associated with at least one of transient enteritis or colitis, cholecystitis, bacteremia, cholangitis, urinary tract infection (UTI), traveler's diarrhea, neonatal meningitis and pneumonia, or any conditions, symptoms or effects associated therewith.

Thus, in some specific embodiments, the biosensor systems, arrays, methods, as well as the kits and devices derived therefrom, in accordance with the present disclosure, may be applicable for transient enteritis. The term “transient enteritis or colitis” relates to an inflammation of the small intestine. It is most commonly caused by food or drink contaminated with pathogenic microbes. Duodenitis, jejunitis and ileitis are subtypes of enteritis which are only localized to a specific part of the small intestine. Inflammation of both the stomach and small intestine is referred to as gastroenteritis. Signs and symptoms of enteritis are highly variable and vary based on the specific cause and other factors such as individual variance and stage of disease. Symptoms may include abdominal pain, cramping, diarrhoea, dehydration, fever, nausea, vomiting and weight loss.

In yet some further embodiments, the biosensor systems, arrays, methods, as well as the kits and devices derived therefrom, in accordance with the present disclosure may be applicable for Cholecystitis. As used herein, Cholecystitis is inflammation of the gallbladder. Symptoms include right upper abdominal pain, nausea, vomiting, and occasionally fever. Often gallbladder attacks (biliary colic) precede acute cholecystitis. Complications of acute cholecystitis include gallstone pancreatitis, common bile duct stones, or inflammation of the common bile duct.

In some further embodiments, the biosensor systems, arrays, methods, as well as the kits and devices derived therefrom, in accordance with the present disclosure may be applicable for Bacteremia. Bacteremia (also bacteraemia) refers to the presence of bacteria in the blood. Bacteria can enter the bloodstream as a severe complication of infections (like pneumonia or meningitis), during surgery (especially when involving mucous membranes such as the gastrointestinal tract), or due to catheters and other foreign bodies entering the arteries or veins (including during intravenous drug abuse). Transient bacteremia can result after dental procedures or brushing of teeth.

Bacteremia can have several important health consequences. The immune response to the bacteria can cause sepsis and septic shock, which has a high mortality rate. Bacteria can also spread via the blood to other parts of the body (which is called hematogenous spread), causing infections away from the original site of infection, such as endocarditis or osteomyelitis.

Still further, in some embodiments, the biosensor systems, arrays, methods, as well as the kits and devices derived therefrom, in accordance with the present disclosure may be applicable for cholangitis. Ascending cholangitis, also known as acute cholangitis or cholangitis, is inflammation of the bile duct, usually caused by bacteria ascending from its junction with the duodenum (first part of the small intestine). It tends to occur if the bile duct is already partially obstructed by gallstones. Characteristic symptoms include yellow discoloration of the skin or whites of the eyes, fever, abdominal pain, and in severe cases, low blood pressure and confusion.

In yet some further embodiments, the biosensor systems, arrays, methods, as well as the kits and devices derived therefrom, in accordance with the present disclosure may be applicable for urinary tract infection. A urinary tract infection (UTI) is an infection that affects part of the urinary tract. When it affects the lower urinary tract, it is known as a bladder infection (cystitis) and when it affects the upper urinary tract it is known as kidney infection (pyelonephritis). Symptoms from a lower urinary tract include pain with urination, frequent urination, and feeling the need to urinate despite having an empty bladder. Symptoms of a kidney infection include fever and flank pain usually in addition to the symptoms of a lower UTI. In some cases, the urine may appear bloody.

In certain embodiments, the biosensor systems, arrays, methods, as well as the kits and devices derived therefrom, in accordance with the present disclosure may be applicable for Traveler's diarrhea. Traveler's diarrhea (TD) is a stomach and intestinal infection. TD is defined as the passage of unformed stool (one or more by some definitions, three or more by others) while traveling. It may be accompanied by abdominal cramps, nausea, fever, and bloating. Occasionally bloody diarrhea may occur. Most travelers recover within four days with little or no treatment. About 10% of people may have symptoms for a week. Bacteria are responsible for more than half of cases. The bacteria enterotoxigenic Escherichia coli (ETEC) are typically the most common except in Southeast Asia, where Campylobacter is more prominent.

In yet some more embodiments, the biosensor systems, arrays, methods, as well as the kits and devices derived therefrom, in accordance with the present disclosure may be applicable for Neonatal meningitis. Neonatal meningitis is a serious medical condition in infants. Meningitis is an inflammation of the meninges (the protective membranes of the central nervous system (CNS)) and is more common in the neonatal period (infants less than 44 days old) than any other time in life and is an important cause of morbidity and mortality globally. Symptoms seen with neonatal meningitis are often unspecific that may point to several conditions, such as sepsis (whole body inflammation). These can include fever, irritability, and dyspnea. The only method to determine if meningitis is the cause of these symptoms is lumbar puncture (LP; an examination of the cerebrospinal fluid). The most common cause of neonatal meningitis is bacterial infection of the blood, known as bacteremia (specifically Group B Streptococci (GBS; Streptococcus agalactiae), Escherichia coli, and Listeria monocytogenes). Delayed treatment of neonatal meningitis may cause include cerebral palsy, blindness, deafness, and learning deficiencies.

In some embodiments, the biosensor systems, arrays, methods, as well as the kits and devices derived therefrom, in accordance with the present disclosure may be applicable for Pneumonia. Pneumonia is an inflammatory condition of the lung affecting primarily the small air sacs known as alveoli. Typically, symptoms include some combination of productive or dry cough, chest pain, fever, and trouble breathing. Bacteria are the most-common cause of community-acquired pneumonia (CAP), with Streptococcus pneumoniae isolated in nearly 50% of cases. Other commonly-isolated bacteria include Haemophilus influenzae in 20%, Chlamydophila pneumoniae in 13%, and Mycoplasma pneumoniae in 3% of cases; Staphylococcus aureus; Moraxella catarrhalis; Legionella pneumophila; and Gram-negative bacilli. A number of drug-resistant versions of the above infections are becoming more common, including drug-resistant Streptococcus pneumoniae (DRSP) and methicillin-resistant Staphylococcus aureus (MRSA).

It should be understood that the diagnostic methods disclosed by the invention may be further used for monitoring subjects treated with any therapeutic compound.

More specifically, the diagnostic methods of the invention may be further used for monitoring the extent of infection (or bacterial load) in the treated subject. For such monitoring purpose, the steps of the methods of the invention may be repeated at least one further time for at least one further sample obtained from the subject. In some embodiments, the sample is obtained in another time point and is therefore considered herein as a temporally separated sample. As indicated above, in accordance with some embodiments of the invention, in order to assess the patient condition, or monitor the disease progression, as well as responsiveness to a certain treatment, at least two “temporally-separated” test samples must be collected from the examined patient and compared thereafter in order to obtain the rate of change in the amount of bacteria between said samples, as reflected by the amount of T3SS component (e.g., the EspB protein) measured and determined by the biosensor systems, arrays, methods, as well as the kits and devices derived therefrom, in accordance with the present disclosure. In practice, to detect a change in at least one of these parameters between said samples, at least two “temporally-separated” test samples and preferably more must be collected from the patient.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

When calculating the rate of change in the amount of the detected T3SS component (e.g., EspB), one may use any two samples collected at different time points from the patient. In some embodiments, at least one of the samples may be obtained before the initiation of an anti-bacterial therapy, and at least one of the samples may be obtained after the initiation of such therapy. To ensure more reliable results and reduce statistical deviations to a minimum, averaging the calculated rates of several sample pairs is preferable. A calculated or average value of a negative rate of change in bacterial load, as reflected by the amount of the T3SS component (e.g., EspB) in the sample, indicates that the subject exhibits a beneficial response to the treatment; thereby monitoring the efficacy of a treatment.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more.

Thus, by providing a diagnostic tool, the present disclosure further provides therapeutic methods involving a diagnostic step. The diagnostic steps therefore provide tailor made methods allowing monitoring the patient for the presence of the T3S pathogen, during the treatment.

In yet some other embodiments, the bacteria may be at least one of EPEC and EHEC.

In other specific embodiments, the methods of the invention are applicable for infectious caused by Enteropathogenic Escherichia coli (EPEC).

In some embodiments, the infections relevant to the method of the invention may be associated with at least one of transient enteritis or colitis, cholecystitis, bacteremia, cholangitis, UTI, traveler's diarrhea, neonatal meningitis and pneumonia, or any condition, symptoms or effects associated therewith, as disclosed herein above.

As indicated herein, the invention provides therapeutic methods involving a diagnostic step using the diagnostic systems, arrays, methods, as well as the kits and devices derived therefrom, in accordance with the present disclosure.

It should be understood that the present disclosure provides systems, arrays, methods as well as any devices and kits derived therefrom, that further provide profiling of the identified pathogen. Specifically profiling the enzymatic activity of the pathogen, thereby providing information and characterizing the pathogenicity and/or virulence of the pathogen. This may be reflected by the ability of the pathogen to express and/or be associated with virulent products.

In some embodiments, the virulence and/or pathogenicity of the pathogen relies on the ability of the pathogen to degrade and/or neutralize anti-bacterial or anti-microbial agents.

Anti-bacterial agent, anti-fungal agent, growth factors, anti-inflammatory agents, vasopressor agents including but not limited to nitric oxide and calcium channel blockers, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid.

The term “antimicrobial agent” as used herein refers to any entity with antimicrobial activity (either bactericidal or bacteriostatic), i.e. the ability to inhibit the growth and/or kill bacteria, for example Gram negative bacteria. An antimicrobial agent may be any agent which results in inhibition of growth or reduction of viability of bacteria by at least about 10%, 20%, 30% or at least about 40%, or at least about 50% or at least about 60% or at least about 70% or more than 70%, for example, 75%, 80%, 85%, 90%, 95%, 100% or any integer between 30% and 70% or more, as compared to in the absence of the antimicrobial agent. Stated another way, an antimicrobial agent is any agent which reduces a population of microbial cells, such as bacteria by at least about 30% or at least about 40%, or at least about 50% or at least about 60% or at least about 70% or more than 70%, or any integer between 30% and 70% as compared to in the absence of the antimicrobial agent. In one embodiment, an antimicrobial agent is an agent which specifically targets a bacteria cell. In another embodiment, an antimicrobial agent modifies (i.e. inhibits or activates or increases) a pathway which is specifically expressed in bacterial cells. An antimicrobial agent can include any chemical, peptide (i.e. an antimicrobial peptide), peptidomimetic, entity or moiety, or analogues of hybrids thereof, including without limitation synthetic and naturally occurring non-proteinaceous entities. In some embodiments, an antimicrobial agent is a small molecule having a chemical moiety. For example, chemical moieties include unsubstituted or substituted alkyl, aromatic or heterocyclyl moieties including macrolides, leptomycins and related natural products or analogues thereof. Antimicrobial agents can be any entity known to have a desired activity and/or property, or can be selected from a library of diverse compounds.

In yet some further embodiments, such antibacterial agents may be antibiotic agents. Still further, in some embodiments such antibiotic agent may be at least one beta-lactam antibiotic agent.

The term “β-lactam” or “β-lactam antibiotics” as used herein refers to any antibiotic agent which contains a b-lactam ring in its molecular structure. β-lactam antibiotics are a broad group of antibiotics that include different classes such as natural and semi-synthetic penicillins, clavulanic acid, carbapenems, penicillin derivatives (penams), cephalosporins (cephems), cephamycins and monobactams, that is, any antibiotic agent that contains a β-lactam ring in its molecular structure. They are the most widely-used group of antibiotics. While not true antibiotics, the β-lactamase inhibitors are often included in this group.

β-lactam antibiotics are analogues of D-alanyl-D-alanine the terminal amino acid residues on the precursor NAM/NAG-peptide subunits of the nascent peptidoglycan layer. The structural similarity between β-lactam antibiotics and D-alanyl-D-alanine prevents the final crosslinking (transpeptidation) of the nascent peptidoglycan layer, disrupting cell wall synthesis. Under normal circumstances peptidoglycan precursors signal a reorganization of the bacterial cell wall and, as a consequence, trigger the activation of autolytic cell wall hydrolases. Inhibition of cross-linkage by β-lactams causes a buildup of peptidoglycan precursors, which triggers the digestion of existing peptidoglycan by autolytic hydrolases without the production of new peptidoglycan. As a result, the bactericidal action of β-lactam antibiotics is further enhanced.

Generally, β-lactams are classified and grouped according to their core ring structures, where each group may be divided to different categories. The term “penam” is used to describe the core skeleton of a member of a penicillin antibiotic. i.e. a β-lactam containing a thiazolidine rings. Penicillins contain a β-lactam ring fused to a 5-membered ring, where one of the atoms in the ring is sulfur and the ring is fully saturated. Penicillins may include narrow spectrum penicillins, such as benzathine penicillin, benzylpenicillin (penicillin G), phenoxymethylpenicillin (penicillin V), procaine penicillin and oxacillin. Narrow spectrum penicillinase-resistant penicillins include methicillin, dicloxacillin and flucloxacillin. The narrow spectrum β-lactamase-resistant penicillins may include temocillin. The moderate spectrum penicillins include for example, amoxicillin and ampicillin. The broad spectrum penicillins include the co-amoxiclav (amoxicillin+clavulanic acid). Finally, the penicillin group also includes the extended spectrum penicillins, for example, azlocillin, carbenicillin, ticarcillin, mezlocillin and piperacillin. Other members of this class include pivampicillin, hetacillin, bacampicillin, metampicillin, talampicillin, epicillin, carbenicillin, carindacillin, tie arcillin, azlocillin, piperacillin, mezlocillin, mecillinam, pivmecillinam, sulbenicillin, clometocillin, procaine benzylpenicillin, azidocillin, penamecillin, propicillin, pheneticillin, cloxacillin and nafcillin.

β-lactams containing pyrrolidine rings are named carbapenams. A carbapenam is a β-lactam compound that is a saturated carbapenem. They exist primarily as biosynthetic intermediates on the way to the carbapenem antibiotics. Carbapenems have a structure that renders them highly resistant to β-lactamases and therefore are considered as the broadest spectrum of β-lactam antibiotics. The carbapenems are structurally very similar to the penicillins, but the sulfur atom in position 1 of the structure has been replaced with a carbon atom, and hence the name of the group, the carbapenems. Carbapenem antibiotics were originally developed from thienamycin, a naturally-derived product of Streptomyces cattleya. The carbapenems group includes: biapenem, doripenem, ertapenem, imipenem, meropenem, panipenem and PZ-601.

β-lactams containing 2,3-dihydrothiazole rings are named penems. Penems are similar in structure to carbapenems. However, where penems have a sulfur, carbapenems have another carbon. There are no naturally occurring penems; all of them are synthetically made. An example for penems is faropenem.

β-lactams containing 3,6-dihydro-2H-1,3-thiazine rings are named cephems. Cephems are a subgroup of b-lactam antibiotics and include cephalosporins and cephamycins. The cephalosporins are broad-spectrum, semisynthetic antibiotics, which share a nucleus of 7-aminocephalosporanic acid. First generation cephalosporins, also considered as the moderate spectrum includes cephalexin, cephalothin and cefazolin. Second generation cephalosporins that are considered as having moderate spectrum with anti-Haemophilus activity may include cefaclor, cefuroxime and cefamandole. Second generation cephamycins that exhibit moderate spectrum with anti-anaerobic activity include cefotetan and cefoxitin. Third generation cephalosporins are considered as having broad spectrum of activity includes cefotaxime and cefpodoxime. Finally, the fourth generation cephalosporins considered as broad spectrum with enhanced activity against Gram positive bacteria and β-lactamase stability include the cefepime and cefpirome. The cephalosporin class may further include: cefadroxil, cefixime, cefprozil, cephalexin, cephalothin, cefuroxime, cefamandole, cefepime and cefpirome. Cephamycins are very similar to cephalosporins and are sometimes classified as cephalosporins. Like cephalosporins, cephamycins are based upon the cephem nucleus. Cephamycins were originally produced by Streptomyces, but synthetic ones have been produced as well. Cephamycins possess a methoxy group at the 7-alpha position and include: cefoxitin, cefotetan, cefmetazole and flomoxef.

β-lactams containing 1,2,3,4-tetrahydropyridine rings are named carbacephems. Carbacephems are synthetically made antibiotics, based on the structure of cephalosporin, a cephem. Carbacephems are similar to cephems but with carbon substituted for the sulfur. An example of carbacephems is loracarbef. Monobactams are b-lactam compounds wherein the β-lactam ring is alone and not fused to another ring (in contrast to most other β-lactams, which have two rings). They work only against Gram negative bacteria. Other examples of monobactams are tigemonam, nocardicin A and tabtoxin.

β-lactams containing 3,6-dihydro-2H-1,3-oxazine rings are named oxacephems or clavams. Oxacephems are molecules similar to cephems, but with oxygen substituting for the sulfur. Thus, they are also known as oxapenams. An example for oxapenams is clavulanic acid. They are synthetically made compounds and have not been discovered in nature. Other examples of oxacephems include moxalactam and flomoxef. Another group of β-lactam antibiotics is the β-lactamase inhibitors, for example, clavulanic acid. Although they exhibit negligible antimicrobial activity, they contain the β-lactam ring. Their sole purpose is to prevent the inactivation of β-lactam antibiotics by binding the β-lactamases, and, as such, they are co-administered with β-lactam antibiotics. β-lactamase inhibitors in clinical use include clavulanic acid and its potassium salt (usually combined with amoxicillin or ticarcillin), sulbactam and tazobactam.

As shown by the present disclosure, in some non-limiting embodiments, the disclosed methods and systems may provide profiling of the bacteria in the sample for nitrocefin, as a beta-lactam antibiotics.

Thus, in some embodiments, the disclosed methods are applicable for profiling the resistance of the pathogens that exist in the sample (and thus in some embodiments, the pathogen caused the infectious disease in the subject) to various antibiotic agents, for example, beta-lactam antibiotics. Antibiotics resistance as used herein refers to the ability of the pathogen to eliminate the antimicrobial agent and/or the ability of bacteria to resist the effects of antibiotics that would normally kill or inhibit their growth. This results in non-effectiveness of the antibiotic agent in killing and/or attenuating the growth of bacteria. In some embodiments, the antibiotics resistance may be a result of acquiring antibiotic resistance genes (e.g., through horizontal gene transfer, and the like). In yet some further embodiments, the disclosed systems, arrays, methods devices and kits of the present disclosure provide means for detecting the activity of various antibiotic resistance genes, and specifically of the enzyme products of such genes (e.g., the enzyme bata-lactamase), thereby profiling the antibiotic resistance of the pathogen identified in the sample or subject.

As indicated above, the present disclosure further encompasses systems, arrays, methods devices and kits that may be applicable for profiling any enzymatic activity that cause and provide the pathogenicity or virulence of the bacteria detected and identified in the sample. Thus, in some embodiments, the disclosed systems, arrays, methods devices and kits may be applicable for profiling and thus providing information that relates to any other virulence factor, in addition or as an alternative to antibiotic resistance. The term “virulent” as used herein means bacteria that can cause a bacterial disease or infection. In some embodiments, virulent bacteria are those that cause a bacterial disease or infection in any subject (e.g., human subject, or any other organism including but not limited to mammal, rodent, bird, fish, reptile, insect or a plant). Typically, virulent bacteria produce “virulence factors”, that include but are not necessarily limited to bacteria proteins that are involved in pathogenic adhesion, colonization, invasion, biofilm formation or immune response inhibitors, or toxins. Examples of virulence genes include, but are not limited to genes encoding toxins (e.g. Shiga toxin and cholera toxin), hemolysins, fimbrial and afimbrial adhesins, proteases, lipases, endonucleases, endotoxins and exotoxins cytotoxic factors, microcins and colicins and also those identified in the art. Additional virulence factors include but are not limited to cytolitic toxins, such as α-hemolysin, β-hemolysin, γ-hemolysin, leukocidin, Panton-Valentine leukocidin (PVL); exotoxins, such as toxic shock syndrome toxin-1 (TSST-1); enterotoxins, such as SEA, SEB, SECn, SED, SEE, SEG, SEH, and SEI, and exfoliative toxins, such as ETA and ETB. More specifically, the term “toxin” as used herein means a substance generated by bacteria, which can be classified as either exotoxin or endotoxin. Exotoxins are generated and actively secreted; endotoxins remain part of the bacteria. Usually, an endotoxin is part of the bacterial outer membrane, and it is not released until the bacterium is killed by the immune system.

It should be appreciated that the present disclosure may further provide a diagnostic-therapeutic kit. Thus, in some embodiments, the biosensor device of the present invention may be provided in a kit together with at least one anti-bacterial agent (e.g. an antibiotic agent) that may provide means for the combined diagnostic and therapeutic method encompassed by the invention. The kit of the present invention may, if desired, be presented in a pack which may contain one or more units of the kit of the present invention.

The terms “treat, treating, treatment” as used herein and in the claims mean ameliorating one or more clinical indicia of disease activity by administering a pharmaceutical composition of the invention in a patient having a pathologic disorder.

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “prevention” as used herein, includes the prevention or postponement of development of the disease, prevention or postponement of development of symptoms and/or a reduction in the severity of such symptoms that will or are expected to develop, preventing the occurrence or reoccurrence of the acute disease attacks. These further include ameliorating existing symptoms, preventing additional symptoms and ameliorating or preventing the underlying metabolic causes of symptoms.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the infectious disease caused by a T3SS expressing MDR bacteria described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention.

More specifically, treatment or prevention includes the prevention or postponement of development of the disease, prevention or postponement of development of symptoms and/or a reduction in the severity of such symptoms that will or are expected to develop. These further include ameliorating existing symptoms, preventing additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

It should be understood that the therapeutic methods of the invention involve any applicable mode of administration. The phrases “systemic administration”, “administered systemically” as used herein mean the administration of a compound, drug or other material other than directly into the central blood system, such that it enters the patient's system and, thus, is subject to metabolism and other like processes. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Systemic administration includes parenteral injection by intravenous bolus injection, by intravenous infusion, by sub-cutaneous, intramuscular, intraperitoneal injections or by suppositories, by patches, or by any other clinically accepted method, including tablets, pills, lozenges, pastilles, capsules, drinkable preparations, ointment, cream, paste, encapsulated gel, patches, boluses, or sprayable aerosol or vapors containing these complexes and combinations thereof, when applied in an acceptable carrier. Alternatively, to any pulmonary delivery as by oral inhalation such as by using liquid nebulizers, aerosol-based metered dose inhalers (MDI's), or dry powder dispersion devices.

By “topical administration” it is meant that the therapeutic methods disclosed herein may be adapted to any mode of topical administration including: epicutaneous, transdermal, oral, bronchoalveolar lavage, ophthalmic administration, enema, nasal administration, administration to the ear, administration by inhalation.

The invention provides methods for treating infectious diseases caused by bacterial infections. As used herein, “disease”, “disorder”, “condition” and the like, as they relate to a subject's health, are used interchangeably and have meanings ascribed to each and all of such terms.

It is understood that the interchangeably used terms “associated” and “related”, when referring to pathologies herein, mean diseases, disorders, conditions, or any pathologies which at least one of: share causalities, co-exist at a higher than coincidental frequency, or where at least one disease, disorder, condition or pathology causes a second disease, disorder, condition or pathology.

By “patient”, “individual” or “subject” it is meant any organism who may be affected by the above-mentioned conditions, and to whom the prognostic methods herein described are desired, including humans. More specifically, in some embodiments, the biosensor systems, arrays, methods, as well as the kits and devices derived therefrom, in accordance with the present disclosure, are applicable for any mammalian subject. By “mammalian subject” is meant any mammal for which the proposed therapy is desired, including human, equine, canine, and feline subjects, most specifically humans.

As used in this specification and the appended claims, the singular forms “a”, “an”, and “the” include plural references unless the context clearly dictates otherwise. Thus, for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric. The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

### EXAMPLES

### Experimental Procedures

### Bacterial Strains

Wild-type (WT) EPEC O127:H6 strain E2348/69 [streptomycin-resistant](WT-SM) and EPEC null mutant (ΔespB) (EPEC-NA), WT EPEC PCX 341 Nled− β lam (Tet resistant) (WT-Tet), EPEC Δespb PCX 341 Nled− βlam (Tet)− red col) (EPEC-Tet) were used in identification and profiling assays.

The bacterial strains were categorized according to their virulence and/or antibiotic resistance, as shown in Table 1:

TABLE 1
Bacterial strains

| | Pathogenic bacteria | Antibiotic resistance |
|---|---|---|
| Bacterial strains | (contain espB) | (β-lactamase) |
| WT-SM | Yes | No |
| EPEC-NA | No | No |
| WT-Tet | Yes | Yes |
| EPEC-Tet | No | Yes |

Briefly, E. coli strains EPEC WT-SM [WT-SM], EPEC null mutant ΔEspB− NA [EPEC-NA], EPEC WT +pNleD-βlam (Tet) [NWT-Tet] and EPEC− ΔEspB +pNleD-βlam (Tet) [EPEC-Tet] cultured in LB medium with antibiotics supplements for overnight at 37° C. in shaker incubator (250 rpm). Subsequent sub-culturing was carried out in pre-incubated 4 mL cell culture medium (DMEM) with antibiotics supplements for 6 hrs at 37° C. and 5% of CO₂. In-between, IPTG was added to all bacterial strains cultures in order to induce expression of β-lactamase. After the incubation, bacterial cells were collected by mild centrifugation at 500 g for 10 min and washed twice with 1×PBS pH 7.2. Then, bacterial cells density quantified by UV-Vis spectrometer by measuring absorbance at 600 nm.

### Electrochemical Biosensor Fabrication

Electrochemical biochips were fabricated and biofunctionalized as previously reported [Porat-Ophir, C.; et al., Ecs Electrochem Lett 2013, 2 (12), G8-G10; Vernick, S.; et al., Gastroenterology 2012, 142 (5), S345-S345; Vernick, S.; et al., Journal of The Electrochemical Society 2011, 158 (1), P1-P4]. More specifically, electrochemical biochips were designed as electrochemical cells with a three-electrode configuration (working electrode counter electrode and a reference electrode) and microfabricated on a p-doped Si/SiO₂ substrate (with 285 nm thermally grown oxide) by a combination of photolithography (to define the electrodes pattern) and sputtering (gold deposition, Ti/Au 10 nm/90 nm). The wafer-scale fabrication yielded 31-32 chips each comprising three gold electrodes (100 nm Au) as well as contact pads. The working electrode diameter was 0.6 mm. On-chip Ag/AgCl reference electrodes (eᵣ) were prepared by electroplating (in an electroplating bath), and the individual chips were finally diced. The generated chips were characterized electrochemically and by scanning electron microscopy, as shown in FIG. 6A.

Electrochemical chips were thoroughly cleaned and activated by immersing 20 min in a solution of 50 mM KOH and 25% of H2O2 followed by thoroughly rinsing with Milli-Q-water.

The mAbs were thiolated by its incubation with Traut's reagent at a molar ratio of 1:15 for 1 hr at room temperature followed by washing with 0.1M phosphate buffer pH 5 to remove the unreacted reagent. Thiolated mAbs were then covalently immobilized onto the gold working electrodes (e_{w}) of the chips by drop-casting. Alternatively, working electrodes (e_{w}) surfaces were modified with ethanolic solution of 5 mM of 11-amnio-undecanothiol by drop-casting and incubated for 18 hrs resulting in the formation of a self-assembled monolayer (SAM) with free amine functional groups. Then, chips were thoroughly rinsed with absolute ethanol followed by Milli-Q-water, and dried under N₂. The EPEC-EspB7 mAb was activated with 0.4M of EDC and 0.1M NHS mixture for 1 hr at RT under mild shaking condition. Then, 3 μl of activated EPEC-EspB7 mAb was loaded on the working electrodes and incubated for 2 hr at RT under dark. Further, mAb-immobilized electrochemical chips (GE-SAM-mAb) were washed thoroughly with 1×PBS and characterized by electrochemical EIS methods.

### Electrochemical Characterization of Chips

The quality of the electroplated Ag/AgCl quasi reference electrode (eᵣ), and of the whole cell were electrochemically characterized. The eᵣ potential demonstrated a linear dependence on the log of the electrolyte (KCl) concentration following the Nernst equation (FIG. 6B).

An example of cyclic voltammetry (CV) for the electrochemical (EC) biochip is presented in FIG. 7A, where four different scan rates were used consecutively. The peak heights increased with increasing scan rates and were linearly proportional to the square root of the scan rates (FIG. 7B), as expected, following the Randles-Sevick equation. In addition, the peak separation was not significantly affected by the scan rate (FIG. 7C).

### Biofunctionalization

Impedimetric immunosensors are based on immobilized antibodies to detect antigens using electrochemical impedance spectroscopy (EIS) on a solid-state electrode. The immobilization strategy of antibodies is of critical significance in the development because it determines the orientation of the antibody on the electrode's surface. The immobilization approach used in the present disclosure is based on the direct covalent attachment of thiolated antibodies to a gold electrode surface. The thiolation reaction was optimized to obtain an average of ˜6 —SH group per antibody by tuning the ratio of reagent to antibody. This fine-tuning enables control of the level of thiolation and ensures that antibody molecules are introduced with a sufficient number of thiols allowing their immobilization. Estimation of introduced sulfhydryl groups was performed by Ellman assay that is used to quantify the number or concentration of thiol groups in a sample. Antibodies were thiolated in order to obtain a firm immobilization via gold-sulfur covalent bond. Traut's reagent (2-Iminothiolane, 2-IT) reacted with antibody primary amines to yield sulfhydryl groups, according to the mechanism shown in FIG. 8A. Estimation of introduced sulfhydryl groups was performed by Ellman assay. Ellman's Reagent (5,5′-dithiobis-(2-nitrobenzoic acid, or DTNB) is used to quantify the number or concentration of thiol groups in a sample. It is very useful as a sulfhydryl assay reagent because of its specificity for —SH groups at neutral pH, high molar extinction coefficient and short reaction time. DTNB reacts with a free sulfhydryl group to yield a mixed disulfide and 2-nitro-5-thiobenzoic acid (TNB). The target of DTNB in this reaction is the conjugate base (R—S⁻) of a free sulfhydryl group. TNB is the “colored” species produced in this reaction and has a high molar extinction coefficient with a value of 14,150M⁻¹cm⁻¹ at 412 nm. The DTNB reduction reaction and its structure are shown in FIG. 8B. Introduced —SH groups were quantified by reference to the extinction coefficient of TNB following:

C=A/bE; where A=absorbance, b=optical path length (cm), E=molar extinction coefficient, and C=concentration (molar). Antibodies incubated with Traut's reagent at a ratio of 1:10 and 1:15, yielded an average —SH groups per antibody of 3.63 and 6.7, respectively.

Gold surfaces can be readily reacted with the sulfur head of thiolated molecules enabling the immobilization of biorecognition molecules. An assessment of the immobilization efficiency was carried out by fluorescence microscopy analysis, using a thiolated fluorescently (Cy3)-labeled antibody compared with non-thiolated antibody. Fluorescence microscopy images shown in FIGS. 9A to 9D, confirm the immobilization of antibodies to the gold electrode. Electrode surface characterization by AFM, as shown in FIG. 9E to 9F, provides further indication for the immobilization of antibodies.

Finally, this direct approach to electrode functionalization is advantageous compared to well-established self-assembled monolayer (SAM) generation methods since it involves a straightforward preparation and avoids complete electrode passivation often attained with SAM. Furthermore, this functionalization procedure can be readily scaled up as it is compatible with microarray printing technology.

### Biosensor Measurements of EspB Binding

Biosensor chips were characterized by volumetric methods (cyclic voltammetry (CV) and electrochemical impedance spectroscopy (EIS) recorded by commercial potentiostat device (BioLogic).

The faradaic current response of a routinely employed redox couple (10 mM K₃Fe(CN)₆) found within the measurement buffer, was monitored both by CV and EIS. The impedance spectra of the freshly cleaned electrodes were obtained prior and post antibody immobilization, with a potential amplitude of 5 mV at a frequency range of 100 kHz to 10 Hz. Charge transfer resistance (Rₑₜ) was determined and analyzed by fitting to a Randles circuit model. The CV was collected within a potential range of −0.2V to +0.5V vs. Ag/AgCl at a scan rate of 100 mV/sec.

### Impedimetric Biosensor for the Detection of EspB Antigen and EspB-Presenting Whole Bacterial Strains

Purified EspB protein solutions (at the concentration of 0, 1, 4, 10, and 250 μg/mL or at the concentration of 0, 0.001, 0.01, 0.1, 1.0, 10.0 and 100 μg/mL in PBS) were incubated for 10 min or 1 h at room temperature on the working electrode. After the incubation, biochips were washed with 1×PBS and EIS spectra were recorded. The specificity of the obtained signals was verified by two control experiments. A negative control that included an unrelated antigen (2 μg/ml of the toxin Microcystin-LR, PubChem CID: 445434) and additionally, a purified EspB antigen (2 μg/mL) without the immobilized mAb (on a nonfunctionalized bare electrode). All Measurements of soluble EspB were repeated 3-6 times for each protein concentration. The charge transfer resistance (R_{ct}) values were obtained by fitting the generated Nyquist plots to a Randles equivalent circuit. The percent change in R_{ct} ratios between the biofunctionalized electrodes and varying EspB concentrations was calculated and averaged from:

$$\sum {R}_{ct} ( EspB ) / {R}_{ct} ( mAb ) - 1.$$

In order to detect whole bacterial cell suspensions, WT-SM, EPEC-NA. WT-Tet and EPEC-Tet bacterial strains were cultured as described herein above, gently centrifuged (500×g, 5 min) and resuspended in PBS to an amount of 5 μL of 10⁶ cells/mL or a maximum of about 5000 cells.

Five microliters of bacteria-containing samples were incubated on the biochip electrode for 10 min, the electrode was then rinsed, and CV and EIS measurements were taken. The percent change in R_{ct} ratios measured was calculated and averaged from 20 repeats (five measurements each containing four samples) for each strain. The mean of the averaged ratios and the standard error of the mean were calculated. Differences between the means were statistically significant as indicated by a t-test using an alpha level of 0.05. In order to compare the means of R_{ct} ratios of both strains, standard errors were combined in quadrature.

All the biochips containing the captured bacteria were collected, and subjected to 30 min sonication in a bath sonicator (under cooling conditions) to break the bacteria cells and release the cellular contents. After sonication, cell lysates were collected and concentrated using 3 kDa Amicon Ultra centrifugal filters (Merck Millipore Ltd. Ireland) by removing excess solvent and impurities by centrifugation at 14,500 rpm for 15 min. The retentates were collected and stored at 4° C. before use. The collected retentates were used for nitrocefin assay to detect and quantify the antibacterial resistant marker β-lactamase.

### Nano Differential Scanning Fluorimetry (NanoDSF)

To assess the thermal stability of mAb-B7, 20 μM mAb-B7 samples were loaded into UV capillaries (NanoTemper Technologies) and analyzed using the nanoDSF Prometheus NT.48. The temperature gradient was set to 1° C./min increase between 15° C. and 95° C. The melting temperatures (Tm) that was derived from protein unfolding was presented by plotting the tryptophan fluorescence at λ=330 nm and λ=350 nm over temperature. The melting temperatures were determined by calculating the maximum of the first derivative and the peak position (at Tₘ) was determined.

### Electrochemical Impedance Spectroscopy (EIS) Measurements

Biosensor measurements were based on Electrochemical Impedance Spectroscopy (EIS) recorded by a commercial potentiostat device (BioLogic, Seyssinet-Pariset, France). EIS was employed to examine the gold electrode before and after modification with the thiolated EspB-specific monoclonal antibody. In a faradaic impedance measurement, a small sinusoidal AC voltage probe is applied while monitoring the current response at different frequencies. The real (resistive) component of the impedance (determined by the in-phase current response) is plotted against the imaginary (capacitive) component (determined by the out-of-phase current response) with respect to frequency. Both are described by

$${Z}^{′} = {R}_{s} + \frac{{R}_{ct}}{1 + {ω}^{2} {R}_{ct}^{2} {C}_{dl}^{2}} and {Z}^{″} = \frac{{R}_{ct}^{2} {C}_{dl} ω}{1 + {ω}^{2} {R}_{ct}^{2} {C}_{dl}^{2}} ,$$

where Rₛ is the solution resistance, Rₜ the charge transfer resistance, C_{dl} is the double-layer capacitance, and ω the angular frequency, which is commonly represented in a Nyquist plot. The impedance results of the thus-obtained Nyquist plot are fitted to an equivalent circuit (Randles circuit) used to interpret the electrochemical system, as shown in the inset of FIG. 10A. An increase in charge transfer resistance (R_{ct}) is attributed to surface adsorption of bound biomolecules.

### The Randles Circuit

To extract the parameter of interest, a generated Nyquist plot is commonly fitted to a model equivalent electronic circuit, Randles circuit. If an analyte affects one of these circuit parameters, then impedance methods can be used for analyte detection. The R_{ct} depicts the opposition experienced to electron movement and it increases in the presence of bound biomolecules. For a one-electron process the R_{ct}, which controls the electron transfer kinetics of Fe(CN)₆^{4-/3-} at the interface of the electrode, can be described by:

$${R}_{ct} = \frac{RT}{{F}^{2} {κ}^{2} C}$$

where R denotes the gas constant, T is temperature, F is Faraday constant, κ⁰ is the electron transfer rate constant and C is the concentration of the electroactive species. The semi-circular region represents a slower charge transfer at higher frequencies whereas the straight line describes a faster mass transfer at lower frequencies. Also, a change in Warburg impedance, Z_{w}, which is dominated by mass transfer can occur when the diffusional transport of electroactive species from the bulk solution to the electrode surface is impeded due to the binding of biomolecules and targets onto the electrode [J. Yeh, B. et al., Sensors Actuators, B Chem. 237 (2016) 329-340]. However, both R_{ct} and Z, depend on the concentration of electroactive species and the applied potential [A. Lasia, Electrochemical Impedance Spectroscopy and its Applications—Andrzej Lasia—Google Books, (2014) https://books.google.com/books?id=lWEgBAAAQBAJ&printsec=frontcover&source=gbs_ge_summary_r&cad=0#v=onepage&q&f=false].

The Nyquist plots arising from EIS measurements were fitted to the following Randles circuit from which the parameter of interest, R_{ct}, values were calculated.

By using an equivalent circuit, the impedance spectra obtained in the measurements were accurately fitted.

### Measurement Platform

A PTFE (Teflon™) measurement platform that enables simultaneous interrogation of multiple chips, provides electrical contacts for the chips and connects to a potentiostat device was designed using Solidworks™ software and machined using CNC milling. The resulted platform contains defined slots for 12 chips with liquid chambers of 300 μl, and also enables hydrodynamic measurements.

Electrochemical detection of Nitrocefin Hydrolysis by β-Lactamase

Nitrocefin is a chromogenic cephalosporin and an analog to β-lactam antibiotics. It is routinely used to detect the presence of β-lactamase enzymes. Nitrocefin assay was used for the detection of β-lactamase from bacterial pathogens to determine the β-lactam antibiotics resistance.

In order to quantify the concentration of 3-lactamase expressed by the bacterial strains, the cell lysates were collected from the bacterial strains and 30-50 μL of cell lysates were incubated with nitrocefin. The reaction mixtures were incubated for 40 min and analyzed by either UV-Vis. Spectrophotometer, Differential pulse voltammetry (DPV), cyclic voltammogram (CV) and/or Square wave voltammetry (SWV).

Similarly, 30-50 μL of the concentrated cells lysates collected from the captured bacteria cells (captured on the biochip in the impedimetric channel) were reacted with nitrocefin for 1 hr. The reaction mixtures were analyzed by either UV-Vis. Spectrophotometer, DPV, CV and/or SWV.

### Electrochemical Detection

Custom fabricated microelectrode chips were used for the electrochemical analysis of nitrocefin assay. The electrode chip contains a gold working electrode, gold counter electrode and Ag/AgCl reference electrode. The electrode chip connected to Palm sense multi-channel potentiostat (MultiEmStat4) (PlamsensBV, Netherland) and accessed through PS Multi-Trace 4.4 software.

### Cyclic Voltammogram (CV)

Nitrocefin substrate 50 μL was loaded on to the electrode surface and cyclic voltammogram (CV) recorded with applied potential from −0.2V to +1.2V with scan rate 0.1V/s. Similarly, nitrocefin hydrolyzed by purified β-Lactamase or by bacterial cell lysates CV were also recorded, and peak currents were analyzed.

### Square Wave Voltammetry (SWV)

Square wave voltammetry (SWV) measurements were carried out with the following parameters: applied potential window: −0.2V to +1.2V; Estep: 0.01V; Amplitude: 0.01V and Frequency: 20 Hz. The nitrocefin specific currents (ΔI) were measured and analyzed.

### Differential Pulse Voltammetry (LPV)

Interestingly, hydrolyzed nitrocefin was found to be electroactive at a potential of ˜+0.92V, where it is electro-oxidized, producing an anodic differential pulse voltammetry (DPV) using a multi-channel potentiostat (Multi EmStat, PlamsensBV, Houten, The Netherland). Hydrolyzed nitrocefin 30 μL were loaded on a cleaned, bare gold electrode, and a differential pulse voltammetry (DPV) analysis was performed at room temperature with the following parameters: Potential scan from −0.2 V to +1.2 V at a scan rate of 0.1 V/s, pulse amplitude of 0.2V, and pulse duration of 20 ms. The peak current signals were recorded and further analyzed,

### Spectrophotometry Detection

7.5 μg nitrocefin were mixed with different concentrations of purified recombinant β-lactamase (0, 1, 2, 3, 4, 5 and 10 ng/mL) and incubated for 40 min in dark at RT. The typical yellow color of nitrocefin changes to cherry red color indicating the hydrolysis of β-lactam ring. The amount of nitrocefin hydrolyzed by β-lactamase was quantified by measuring absorbance maximum at 490 nm using UV-Vis. spectrophotometer.

### Example 1

### Development of the Identification, Quantification, Capturing Unit

The inventors herein present the development of a bioelectronic diagnostic device that combines the ability to detect both the EspB pathogenic protein marker, a component of the T3SS of virulent enteropathogens, either secreted or in the context of a whole bacteria cell, and also β-lactam antibiotic resistance via detection of the enzyme β-lactamase. This dual detection is enabled by integrating an electrochemical microchip that contains two different measurement channels with a highly specific mAb (mAb-EspB-B7) and with the cephalosporin substrate, nitrocefin. The mAb-EspB-B7 demonstrates an exceptional binding capacity to soluble EspB and in the context of whole bacteria, and a high stability under a variety of conditions, allowing its robust integration with an electrochemical chip, and enabling the transduction of EspB binding into a measurable current response, using EIS. Nitrocefin can be integrated with an electrochemical chip since its enzyme-catalyzed product is highly electroactive and used as a marker for the detection of β-lactamase by a voltammetry measurement.

As a proof of concept, the inventors developed the mAb-EspB-B7-based biosensor, as the first unit that provides identification, quantification and capturing the target in a sample (EspB protein and EspB-presenting bacteria).

The potential of the developed mAb-EspB-B7 in diagnostic applications may be exploited by integrating it with an electrochemical biosensor. In particular, impedimetric immunosensors show great promise in rapidly detecting low concentrations of target antigens within a highly simplified testing setup. A general scheme of the disclosed device is shown in FIG. 4.

FIG. 4, and FIG. 1A show an electrochemical chip device providing an electrode arrangement within contact with a sample. The sample is provided by a sample collector and may be pushed into a measurement chamber using a syringe/plunger. The inventors sought to demonstrate this potential by constructing a miniature electrochemical biochip functionalized with mAb-EspB-B7, as illustrated in FIGS. 5A and 10A. The working electrode of the electrode arrangement was pre-treated by biofunctionalization with mAb-EspB-B7. Impedance measurement between the electrodes provides data indicative of agents bound to the binding site. The impedance measurements may be represented by Nyquist plots that were fitted to an equivalent model circuit from which charge transfer resistance (R_{ct}) values were obtained. The EIS was recorded for the electrodes before and after antibody immobilization and these were compared with measurements taken after a 10 min incubation of purified EspB protein at varying concentrations. Using the Nyquist plot (FIGS. 10B and 11A) the effect of mAb-EspB-B7 immobilization on the R_{ct} was observed. In a bare electrode, the resistance to charge transfer is small and impedance is dominated by mass transfer (diffusion of the electroactive species), the so-called Warburg impedance, which is evident in low frequencies [Lasia, A., in Electrochemical Impedance Spectroscopy and its Applications, Springer New York: New York, NY, 2014; pp 85-125]. The Warburg impedance is considerably decreased following self-assembled monolayer (SAM) modification and/or mAb immobilization as mass transfer is no longer a significant factor. Instead, the contribution of R_{ct} to the impedance is now largely dominant, as an insulating layer of biomolecules is attached to the surface. Following a brief incubation of 250 μg/mL purified EspB solution, a significant increase in R_{ct} was observed, which is directly correlated with the bound antigen concentration further adding to the resistive component of the impedance (FIGS. 9B and 11A). It was found that the addition of purified EspB protein affects the R_{ct} in a dose-dependent manner, enabling the distinction between different concentrations (FIGS. 10C and 11B). In addition, incubation of a non-specific antigen (microcystin toxin (MCLR)) showed no effect on the measured R_{ct} (FIG. 9C). Similarly, no effect was observed from purified EspB protein directly incubated on a bare electrode, indicating that the change in R_{ct} reflects the specific binding of EspB to the mAB-EspB-B7 antibody (FIGS. 10C and 11B). Finally, detection sensitivity of this method was optimized and it was found to be 1 ng/mL of EspB. The relative change in R_{ct} exhibits an exponential dependence on concentration, as seen in FIGS. 10D and 11B.

To examine the applicability of mAb-EspB-B7-based electrochemical bio-sensing in detecting whole bacteria harboring T3SS (and present EspB), WT EPEC (also named as WT-SM), and their ΔespB mutant strains were grew under T3SS-inducing conditions and incubated on the biochip. As shown in FIG. 10E, higher R_{ct} values were consistently recorded for EPEC WT (mean R_{ct} change=1.22±0.09) compared to the ΔespB mutant strain (mean R_{ct} change=0.86±0.12). The minimal binding of the ΔespB strain was likely due to nonspecific adsorption. It should be noted that following centrifugation and lack of T3SS-inducing conditions, shedding of the complex is likely to occur. Nevertheless, the obtained signals were shown to be significantly higher (36%±15) in response to EPEC WT compared to the ΔespB strain (p=0.03).

Similarly, as shown in FIG. 12A and in the corresponding histogram of FIG. 12B, higher R_{ct} values were consistently recorded for WT-SM allowed by WT-Tet compared to the ΔEspB mutant strains, EPEC-NA and EPEC-Tet. The minimal binding of the ΔEspB strain is likely due to nonspecific adsorption. The obtained immuno-impedimetric signals, collected from multiple samples, were highly reproducible and showed a significantly (˜2-3 times) higher R_{ct} in response to WT-SM (ΔR_{ct}=˜85%) and WT-Tet (ΔR_{ct}=˜52%) compared to the ΔEspB strains EPEC-NA and EPEC-Tet (ΔR_{ct}=˜20-25%).

Overall, these results demonstrated the potential of the biosensor of the present disclosure, to detect EspB, both in its secreted form as well as an integral component of the assembled T3SS complex in the context of whole bacteria.

### Example 2

### Development of the Profiling Unit

As noted above, and illustrated in FIGS. 4 and 5B, the dual-mode electrochemical platform of the present disclosure comprises a system comprising two units, specifically, two electrochemical biochips, each configured as an individual electrochemical cell array equipped with a multiplicity of microelectrodes. The electrochemical chips, which are enclosed in separate chambers, are connected by a microfluidic channel enabling a sequential measurement of a sample. The platform also contains a miniaturized ultrasonic transducer, a filtering unit, a waste chamber, and a potentiostat circuit (FIG. 4). As a proof of concept, the second unit of the disclosed system was configured for profiling of enzymatic activity of the identified pathogen in the sample, and specifically, antibiotic resistance of the EspB expressing pathogen. More specifically, this platform was applied in the detection of EspB-presenting EPEC that also possess antibiotic resistance by expressing ESBL enzyme (extended spectrum β-lactamase). Thus, the platform provides quantitative determination of the pathogen and characterization thereof, specifically, enzymatic profiling.

For preparation of the second unit that provides enzymatic profiling, for example, antibiotic resistance data, nitrocefin, that is a substrate of β-lactamase enzyme was loaded on the surface of the working electrode, thereby creating a profiling biosensor. Nitrocefin is a chromogenic cephalosporin substrate that contains β-lactam, nitrobenzene and thiazolidine rings. β-lactamase enzyme hydrolyses the amide bond between carbonyl carbon and nitrogen in β-lactam ring producing free carboxylic and amine group (FIG. 5C).

### Example 3

### Standardization of β-Lactamase Detection by a Colorimetric Nitrocefin Assay

The inventors have initially tested the ability to monitor β-lactamase activity by performing the colorimetric nitrocefin assay. Enzymatic hydrolysis of nitrocefin is accompanied by a visible shift in the UV absorbance from yellow (substrate) to cherry red (hydrolyzed product). A standard calibration curve was thus prepared using different β-lactamase concentrations and nitrocefin substrate. Red color intensities were gradually increased while increasing the concentration of β-lactamase from 0 to 100 ng/mL. The UV-visible spectra in FIG. 13A shows the gradual decrease of absorption peak at 380 nm and the appearance of a new peak at 490 nm. The absorbance intensity at 490 nm was gradually increasing with respective enzyme concentrations. The standard β-lactamase calibration curve in FIG. 13B highlights the dependence of the absorbance peak at 490 nm on enzyme concentrations.

Next, a similar colorimetric nitrocefin assay was carried out by using the cell lysates of the four experimental bacteria strains in order to determine their β-lactamase expression. As shown in the UV-vis. spectra in FIG. 14A, the hydrolyzed nitrocefin absorbance peak at 490 nm appears only in the cell lysates of antibiotic-resistant bacteria strains [WT-Tet and EPEC-Tet] whereas the cell lysates of WT-SM and EPEC-NA, which do not express β-lactamase, only present the absorbance peak of nitrocefin substrate at 380 nm. The bar graph in FIG. 14B summarizes the optical absorbance peak intensities recorded at 490 nm for the nitrocefin assay with the different bacteria cell lysates (average measurements of 9 experiments). The peak intensities of hydrolyzed nitrocefin obtained by antibiotic-resistant bacteria cell lysates were 11 times higher than these obtained by the antibiotic sensitive bacteria cell lysates, and 14 times higher compared with a nitrocefin substrate used as control. Finally, β-lactamase concentration in WT-Tet and EPEC-Tet bacteria cell lysate was calculated and it was found to be 15.9 μg/mL and 15.3 μg/mL, respectively.

### Example 4

### Electrochemical Detection of β-Lactamase Using the Biosensor

Nitrocefin contains nitrobenzene and thiazolidine groups, which undergo irreversible oxidation at applied potentials >+0.8V. This electroactivity was utilized in the detection of β-lactamase by employing a voltametric analog of the nitrocefin assay.

Nitrocefin hydrolysis (nitrocefin specific currents (ΔI)) by purified β-Lactamase (FIGS. 15 and 16), were measured and analyzed using cyclic voltammogram (CV) (recorded with applied potential from −0.2V to +1.2V with scan rate 0.1V/s), or by Square wave voltammetry (SWV) measurements (with the following parameters: applied potential window: −0.2V to +1.2V; Estep: 0.01V; Amplitude: 0.01V and Frequency: 20 Hz) (FIGS. 15 and 17) or by differentia DPV analysis (FIGS. 16 and 18).

A CV analysis showed no specific oxidation peak for Nitrocefin. however, following the addition of β-lactamase enzyme to nitrocefin substrate, the β-lactam ring was hydrolyzed by breaking amide bond thus increasing electronegativity. The hydrolyzed nitrocefin was filtered (using 3 kDa centrifuge filter). The filtered hydrolyzed nitrocefin product was then analyzed by CV and SWV methods. FIG. 15A presents CV of hydrolyzed nitrocefin and shows strong irreversible oxidation peak potential at +1.05V with high current intensity (88.15 μA)—almost 15 times higher than nitrocefin substrate. This result confirms that nitrocefin has gained high electroactive property after the hydrolysis of β-lactam ring. This electroactivity is further revealed by a SWV analysis, as seen in FIG. 15B. The SWV of hydrolyzed nitrocefin shows three distinct current peaks and high intensity peak (ΔI=6.5 μA) was observed at +1.009V, which was 5 times higher than the nitrocefin control. Another peak potential at +0.67 with current intensity (ΔI=4.0 μA), which was two times higher than control. A third peak was observed at lower potential at +0.32V with current intensity (ΔI=1.0 μA), which was not found in nitrocefin control. This peak originates from adsorbed specifies of hydrolyzed nitrocefin.

A DPV analysis (FIG. 16) of nitrocefin substrate yielded a residual oxidation peak at +0.8V with a relatively low current of 49 μA. However, after the hydrolysis of the nitrocefin β-lactam ring, catalyzed by β-lactamase, the oxidation current intensity was dramatically increased and peak potential slightly shifted to +0.92V. Increasing β-lactamase concentrations (0 ng-100 ng/mL) in the reaction solution resulted in increasing current signals at +0.92V until reaching a maximum current signal of 478 μA in response to the addition of 100 ng/mL of β-lactamase, as shown in the DPV voltammogram in FIG. 16A. The measured peak current signals at +0.92V were used to prepare an electrochemical standard calibration curve for β-lactamase shown in FIG. 16B.

### Example 5

Electrochemical Detection of β-Lactamase from Bacteria Cell Lysates

The electrochemical detection of β-lactamase activity was carried out by measuring the electro-oxidation of hydrolyzed nitrocefin product in the different bacteria cell lysates using CV, SWV or DPV analysis.

FIG. 17A discloses SWV analysis and shows two current peaks in the cell lysate of WT EPEC pCX341 Nled-β-lam (Tet) with nitrocefin, the first at +1.02V demonstrating high current signal (ΔI=38 μA) and the second at +0.66V (ΔI=2 μA). Similar peaks were found for EPEC ΔespB pCX341 NleD-β-lam (Tet) cell lysate with nitrocefin. On the other hand, very low current signals were obtained by both bacterial cell lysates that don't express NleD-β-lam with nitrocefin and nitrocefin control (ΔI=2.9-4.3 μA at +1.02V). FIG. 17B discloses a bar graph that compares the measured current signals (ΔI) at +1.02V for all bacteria cell lysates with nitrocefin and nitrocefin control. Similar measurements were performed by CV analysis. The peak currents difference in the CV and SWV results of both antibiotic resistant bacteria cell lysates is due the interference of other cellular molecules in diffusion coefficient of nitrocefin. SWV measurement were performed faster than CV analysis, other cellular molecules from EPEC ΔespB pCX341 NleD-β-lam (Tet) cell lysate may hinder the transport of nitrocefin.

These experimental results support that electrochemical detection of β-lactamase enzyme producing antibiotic resistant bacteria from various sources in short period of time (1 hr) with high sensitivity and more specificity.

FIG. 18 shows a DPV analysis of the β-lactamase activity of WT-Tet, EPEC-Tet, WT-SM and EPEC-NA. Hydrolyzed nitrocefin specific oxidation current peak was observed only in the lysates of both antibiotic-resistant bacteria [WT-Tet and EPEC-Tet] but not in the cell lysates of antibiotic sensitive bacteria [WT-SM and EPEC-NA], as shown in FIG. 18A. Very high current signals with an average of ˜480 μA were observed in a DPV analysis of nitrocefin with a lysate of WT-Tet, which were 1.5 times higher than these obtained by EPEC-Tet cell lysate, suggesting a higher β-lactamase activity in WT-Tet strain. Conversely, current signals that are 12.5 to 25 times lower were recorded in the DPV analysis of the antibiotic sensitive bacteria [WT-SM and EPEC-NA]. The average current signals at +0.92V that were measured with the different bacteria cell lysates are summarized in FIG. 18B bar graph (average of 9 experiments). Finally, the β-lactamase calibration curve was used to determine the concentration of β-lactamase in WT-Tet and EPEC-Tet cell lysates, and it was found to be 21.5 μg/mL and 11.42 μg/mL, respectively.

### Example 6

### Dual Mode EC Detection of Antibiotic-Resistant Pathogenic EPEC

After calibrating each channel individually and determining the performance characteristics of both methods, the biosensor was used in a sequential measurement: immuno-impedimetric detection of pathogens using antibody-functionalized microelectrodes (chip 1) followed by a voltametric detection of antibiotic resistance (chip 2), performed on a single sample.

Following exposure of chip 1 to each bacteria strain and measurement of their impedance spectra, as before, the chip-captured bacteria were collected and lysed by sonication. Cell lysates of each captured strain were reacted with nitrocefin and subsequently measured by DPV on chip 2, as shown in the schematic illustration in FIGS. 4 and 5.

Nitrocefin assay reaction mixtures were analyzed by both UV-Vis. Spectrophotometry and DPV. As seen in FIG. 19A spectra, the absorbance maximum at 390 nm, corresponding to nitrocefin substrate, was found in all samples. However, none of the cell lysates have shown any absorbance of hydrolyzed nitrocefin (490 nm). In fact, all four strains exhibited a similar background absorbance at 490 nm, as shown in FIG. 19B bar graph. These findings indicate that the sensitivity of the optical assay is insufficient to detect β-lactamase activity in the cell lysates of the chip-captured bacteria. Indeed, the number of chip-captured bacteria cells is expected to be low (considering that a maximum number of ˜1,000 cells were loaded on the chip).

The same reaction mixtures were used for electrochemical detection of β-lactamase using DPV. As expected, the highest specific peak current (I=˜160 μA at ˜+0.92V) was found only in WT-Tet, the EspE-presenting strain that expresses β-lactamase. The specific peak currents measured from cell lysates of the other chip-captured strains (WT-SM, EPEC-NA and EPEC-Tet) were significantly lower (˜80%), as shown in FIG. 19C voltammogram. The DPV results clearly indicate that the activity of β-lactamase in WT-Tet can be easily measured by monitoring the oxidation current of its hydrolyzed nitrocefin product whereas no current signals is measured in other strains that do not express the enzyme. The DPV results were averaged and presented in the bar graph in FIG. 19D. As shown, the current signal measured from WT-Tet was higher by nearly an order of magnitude than that recorded for nitrocefin control, ˜7 times higher than EPEC-NA and EPEC-Tet, and ˜3 times higher than WT-SM. Finally, the amount of β-lactamase found in the WT-Tet cell lysate was determined and it was found to be ˜25 ng/mL.

β-Lactamase producing bacterial strains WT-Tet and their controls WT-SM, EPEC-NA and EPEC-Tet. Error bar represents SEM, n=9.

It should be noted that WT-SM is an EspB-presenting bacteria, therefore, successfully captured by chip 1 (GE-SAM-mAb chip), likely affecting a non-specific signal in chip 2. Conversely, EPEC-NA and EPEC-Tet strains do not produce EspB antigen, therefore they were not successfully captured by chip 1 and produced very low current signals.

Finally, WT-Tet bacterial stain expresses both EspB antigen and β-lactamase enzyme, thus possessing both pathogenicity and antibiotic resistance. Therefore, it was successfully captured by GE-SAM-mAb biochip and its cells lysate contains β-lactamase which hydrolyzes nitrocefin producing high peak current signals in DPV.

This developed and integrated electrochemical based-impedimetric and DPV biosensor has ability to detect pathogenic bacterial strain using virulence factor EspB and their β-lactam antibiotic degrading enzyme β-lactamase with high specificity and in short period of time.

1-50. (canceled)

51. A system comprising:
(a) a pathogen identification and/or quantification unit comprising a first electrode arrangement comprising at least one first working electrode located within at least one first chamber; and
(b) a profiling unit comprising a second electrode arrangement comprising at least one second working electrode located within at least one second chamber, said second chamber is connected to said pathogen identification unit to allow selective fluid transmission from said at least one first chamber to said at least one second chamber;
wherein said at least one first working electrode is contacted directly or indirectly to at least one target binding site and/or moiety specific for binding one or more target pathogens; and
wherein said at least one second working electrode is in the vicinity of, or is connected directly or indirectly to at least one substrate molecule/s, wherein interaction of said at least one substrate molecule/s with at least one catalytic macromolecule, catalyzes the production of at least electroactive product.

52. The system of claim 51, wherein one of:
(a) the system further comprising a disruptor associated with said pathogen identification unit and adapted to selectively apply disruption conditions onto one or more target pathogens bound to, or captured by, said target binding site and/or moiety in said first chamber, thereby generating pathogen lysates; or
(b) system further comprising a disruptor associated with said pathogen identification unit and adapted to selectively apply disruption conditions onto one or more target pathogens bound to, or captured by, said target binding site and/or moiety in said first chamber, thereby generating pathogen lysates, wherein said disruptor is an ultrasonic transducer configured for generating and directing an ultrasonic signal onto said at least one first chamber thereby lysing one or more target pathogens bound to, or captured by, said target binding site and/or moiety.

53. The system of claim 51, wherein one of:
(a) said first electrode arrangement is connectable to a detection circuit adapted for electrical detection of at least one target pathogen bound to, or captured by, said at least one target binding site and/or moiety; or
(b) said first electrode arrangement is connectable to a detection circuit adapted for electrical detection of at least one target pathogen bound to, or captured by, said at least one target binding site and/or moiety, wherein said detection circuit is adapted for electrochemical impedance spectroscopy (EIS) for detecting the presence and/or quantity of said at least one target pathogen bound to, or captured by said at least one target binding site and/or moiety of said at least one first working electrode.

54. The system of claim 51, wherein at least one of:
(a) said target pathogen is at least one pathogen expressing, or associated with, at least one component of the Type III Secretion System (T3SS);
(b) said at least one target binding site and/or moiety is comprised within at least one antibody that recognizes and binds at least one component of the T3SS, or any combination or complex thereof, said antibody or any functional fragments thereof is immobilized to said at least one first working electrode;
(c) said component of said T3SS is at least one of the EPEC secreted protein B (EspB), Enteropathogenic Escherichia coli (EPEC) secreted protein A (EspA), and EPEC secreted protein D (EspD), or any fragments or peptides thereof, and any combination or complex thereof; and
(d) said at least one antibody recognizes and binds the EspB protein.

55. The system of claim 54, wherein said at least one antibody recognizes and binds the EspB protein, said antibody comprises a heavy chain complementarity determining region (CDRH) 1 comprising the amino acid sequence GFTFSHYA, as denoted by SEQ ID NO. 6, CDRH2 comprising the amino acid sequence INSNGDST, as denoted by SEQ ID NO. 7, CDRH3 comprising the amino acid sequence ARDRRAGYFDYW, as denoted by SEQ ID NO. 8, and a light chain complementarity determining region (CDRL) 1 comprising the amino acid sequence RDNIGKNY as denoted by SEQ ID NO. 9, a CDRL2 comprising the amino acid sequence RNN as denoted by SEQ ID NO. 10, and a CDRL3 comprising the amino acid sequence SAWDTSLNA as denoted by SEQ ID NO. 11, or any derivative, variant and biosimilar thereof.

56. The system of claim 51, wherein at least one of:
(a) said second electrode arrangement is connected to, or is in the vicinity of, said one or more substrate molecules such that each of the one or more second working electrode/s is in the vicinity of, or is connected directly or indirectly to a single type of substrate molecule, said electrode arrangement is comprised within one or more second chambers;
(b) said profiling unit comprises one or more second chambers, each comprising a respective second electrode arrangement comprising one or more second working electrodes in the vicinity of, or connected directly or indirectly to one or more substrate molecules respectively, wherein each of said one or more second chambers comprises a single type of substrate molecules, and wherein said substrate molecules differ between said one or more second chambers; and
(c) wherein interaction of said substrate molecule/s with at least one catalytic macromolecule, leads to conversion of said substrate into at least one electroactive product, and wherein said second electrode arrangement is connectable to a voltametric circuit adapted to provide voltametric measurement data indicative of production of said electroactive product, thereby indicating the presence of said at least one catalytic macromolecule in said pathogen lysates.

57. The system of claim 51, wherein said catalytic macromolecule is at least one enzyme, wherein said substrate molecule is a specific substrate of said enzyme, and wherein said enzyme catalyzes the conversion of said substrate molecule to at least one electroactive product.

58. The system of claim 57, wherein at least one of:
(a) the presence and/or association of said enzyme in the target pathogen is indicative of the pathogenicity of said target pathogen;
(b) said pathogenicity comprises antibiotic resistance and wherein said enzyme provides directly or indirectly antibiotic resistance to said target pathogen;
(c) said enzyme is at least one of: at least one hydrolase, at least one transferase, and at least one oxidoreductase;
(d) said at least one hydrolase comprises at least one β-Lactamase, at least one macrolide esterase and at least one epoxide hydrolase;
(e) said enzyme is β-Lactamase and wherein said substrate is β-lactam antibiotics or any substrate hydrolyzed by β-Lactamase to produce at least one electroactive product; and
(f) said substrate is Nitorcefin, and wherein hydrolysis of Nitorcefin produces an electroactive product, thereby indicating the presence of β-Lactamase in said target pathogen or pathogen lysates.

59. The system of claim 51, wherein said pathogen identification unit comprises at least one of:
an input port for inserting input sample;
a waste port for rinsing out remaining of said input sample, thereby enabling separation of a pathogen bound to said target binding site and/or moiety;
a filtering unit; and
an output port for selectively allowing fluid transmission to said profiling unit.

60. The system of claim 51, further comprising a controller connectable to one or more valves and said first and second electrode arrangement; said controller is configured and operable to perform at least one of:
(a) be responsive to data indicating input of a sample to said pathogen identification unit; in response to said data, the controller operates said first electrode arrangement for detecting and/or quantifying one or more target pathogens bound to, or captured by, said target binding site and/or moiety;
(b) operation of a waste valve for rinsing out remains of said sample;
(c) operation of said disruptor to apply disruption conditions thereby generating lysates of said target pathogen;
(d) operation of a transmission valve to transmit said pathogen lysates to said profiling unit; and
(e) operation of said second electrode arrangement for detecting at least one electroactive product of said one or more substrate molecules, and generation of output data indicative of catalytic activity profile of at least one catalytic macromolecule of said one or more target pathogens, in accordance with the appearance of at least one electroactive product of said substrate molecules.

61. The system claim 51, comprising an arrangement of one or more identification units, each comprising a first chamber and first electrode arrangement, wherein working electrodes of said first electrode arrangement are connected directly or indirectly to respective one or more target binding moieties, different for each of said one or more identification units; and respective one or more profiling units, each configured to receive pathogen lysates from a respective identification unit, to thereby determine catalytic activity profile of one or more target pathogens, in accordance with respective interactions of catalytic macromolecules in the pathogen lysates with one or more substrate molecules of said at least one profiling unit.

62. An array comprising plurality of systems, each of said systems is as defined by claim 51, and comprises:
(a) a pathogen identification and/or quantification unit comprising a first electrode arrangement comprising at least one first working electrode located within at least one first chamber; and
(b) a profiling unit comprising a second electrode arrangement comprising at least one second working electrode located within at least one second chamber, said second chamber is connected to said pathogen identification unit to allow selective fluid transmission from said at least one first chamber to said at least one second chamber;
wherein said at least one first working electrode is contacted directly or indirectly to at least one target binding site and/or moiety specific for binding one or more target pathogens; and
wherein said at least one second working electrode is in the vicinity of, or is connected directly or indirectly to at least one substrate molecule/s, wherein interaction of said at least one substrate molecule/s with at least one catalytic macromolecule, catalyzes the production of at least electroactive product.

63. A method for identifying, quantifying and/or catalytically profiling one or more target pathogens in at least sample, the method comprising:
(a) contacting said sample with at least one first electrode arrangement comprising at least one first working electrode, or with any unit or system thereof, said at least one first working electrode is connected directly or indirectly to at least one target binding site and/or moiety for binding and/or capturing one or more target pathogens present in said sample;
(b) performing an electrochemical impedance spectroscopy (EIS) analysis of said sample; wherein impedance variations indicate the presence and/or quantity of said target pathogen in said sample;
(c) applying disruption conditions to a target pathogen bound to, or captured by, the target binding site and/or moiety thereby generating target pathogen lysates;
(d) contacting said target pathogen lysates with one or more substrate molecules connected directly or indirectly to or in the vicinity of, one or more second working electrodes of at least one second electrode arrangement, or with any unit or system thereof, wherein said at least one substrate molecule is a substrate of at least one catalytic macromolecule, and wherein said catalytic macromolecule catalyzes the formation of at least one electroactive product using said substrate molecule;
(e) performing an electrochemical voltammetry or amperometry analysis of said sample to detect the production of at least one electroactive product; and
wherein the detection of said product indicates the presence and/or activity of said catalytic macromolecule in said target pathogen lysates, thereby profiling the catalytic activity of said target pathogen in said sample.

64. The method of claim 63, wherein at least one of:
(a) performing an EIS analysis of said sample comprises at least one of faradic EIS and non-faradic EIS;
(b) performing an EIS analysis of said sample, comprising:
(i) contacting said sample with at least one first working electrode, at least one first reference electrode, and at least one first counter electrode, said at least one first working electrode is connected directly or indirectly to at least one target binding site and/or moiety;
(ii) measuring electrical currents between said at least one first working electrode and said at least one first reference electrode in response to alternating electric voltages at different frequencies applied between said at least one first working electrode and said at least one first counter electrode;
(iii) determining electrical impedances based on the measured electrical current and the electric voltages applied at the different frequencies;
(iv) determining a charge transfer electrical resistance based on the determined impedances; and determining presence and/or quantity of said at least one target pathogen captured to said at least one target binding site and/or moiety in accordance with said charge transfer electrical resistance; and
(c) performing an electrochemical voltammetry or amperometry analysis of said sample comprises:
(i) applying voltage signal between said at least one second working electrode and at least one reference electrode and determining electrical current through said at least one second working electrode in response to varying voltage signal; and
(ii) determining peak current value, said peak current value is inversely indicative of presence and/or quantity of said at least one electroactive product.

65. The method of claim 63, wherein at least one of:
(a) said target pathogen is at least one pathogen expressing, or associated with, at least one component of the Type III Secretion System (T3SS);
(b) said target pathogen is at least one pathogen expressing, or associated with, at least one component of the Type III Secretion System (T3SS), wherein said at least one target binding site and/or moiety is comprised within at least one antibody that recognizes and binds at least one component of the T3SS, or any combination or complex thereof, said antibody or any functional fragments thereof is immobilized to said at least one first working electrode;
(c) wherein said component of said T3SS is at least one of the EspB, EspA, and EspD, or any fragments or peptides thereof, and any combination or complex thereof; and
(d) wherein said at least one antibody recognizes and binds the EspB protein.

66. The method of claim 65, wherein said at least one antibody recognizes and binds the EspB protein, said antibody comprises a CDRH1 comprising the amino acid sequence GFTFSHYA, as denoted by SEQ ID NO. 6, CDRH2 comprising the amino acid sequence INSNGDST, as denoted by SEQ ID NO. 7, CDRH3 comprising the amino acid sequence ARDRRAGYFDYW, as denoted by SEQ ID NO. 8, and a CDRL1 comprising the amino acid sequence RDNIGKNY as denoted by SEQ ID NO. 9, a CDRL2 comprising the amino acid sequence RNN as denoted by SEQ ID NO. 10, and a CDRL3 comprising the amino acid sequence SAWDTSLNA as denoted by SEQ ID NO. 11, or any derivative, variant and biosimilar thereof.

67. The method of claim 63, wherein at least one of:
(a) said pathogen is a bacterial pathogen, said bacteria is at least one Multiple Drug Resistant (MDR) bacteria;
(b) said pathogen is a bacterial pathogen, said bacteria is at least one Multiple Drug Resistant (MDR) bacteria, said MDR bacteria is at least one of Enteropathogenic Escherichia coli (EPEC) and Enterohemorrhagic Escherichia coli (EHEC);
(c) said sample is biological sample or an environmental sample;
(d) said catalytic macromolecule is at least one enzyme, wherein said substrate molecule is a specific substrate of said enzyme, and wherein said enzyme catalyzes the conversion of said substrate molecule to at least one electroactive product;
(e) said catalytic macromolecule is at least one enzyme, wherein said substrate molecule is a specific substrate of said enzyme, and wherein said enzyme catalyzes the conversion of said substrate molecule to at least one electroactive product, wherein at least one of:
(i) the presence and/or association of said enzyme in the target pathogen is indicative of the pathogenicity of said target pathogen;
(ii) said pathogenicity comprises antibiotic resistance and wherein said enzyme provides directly or indirectly antibiotic resistance to said target pathogen;
(iii) said enzyme is at least one of: at least one hydrolase, at least one transferase, and at least one oxidoreductase;
(iv) said at least one hydrolase comprises at least one β-Lactamase, at least one macrolide esterase and at least one epoxide hydrolase;
(v) said enzyme is β-Lacatamase and wherein said substrate is β-lactam antibiotics or any substrate hydrolyzed by β-Lactamase to produce at least one electroactive product;
(vi) said substrate is Nitorcefin, and wherein hydrolysis of Nitorcefin produces an electroactive product, thereby indicating the presence of β-Lactarnase in said target pathogen or pathogen lysates; and
(f) wherein said method is for the diagnosis of an infectious condition caused by or associated with at least one T3SS expressing pathogen, in a subject, and for profiling antibiotic resistance of said pathogen in said subject.

68. A method for diagnosing an infectious disease caused by at least one pathogen in a subject, the method comprising the step of detecting, identifying, quantifying and/or catalytically profiling one or more target pathogens in at least sample of said subject, the method comprising:
(a) contacting said sample with at least one first electrode arrangement comprising at least one first working electrode, or with any unit or system thereof, said at least one first working electrode is connected directly or indirectly to at least one target binding site and/or moiety for binding and/or capturing one or more target pathogens present in said sample;
(b) performing an electrochemical impedance spectroscopy (EIS) analysis of said sample; wherein impedance variations, indicate the presence and/or quantity of said target pathogen in said sample;
(c) applying disruption conditions to the target pathogen/s bound to, or captured by, the target binding site and/or moiety thereby generating target pathogen lysates;
(d) contacting said target pathogen lysates with one or more substrate molecules connected directly or indirectly to, or in the vicinity of, one or more second working electrodes of at least one second electrode arrangement, or with any unit or system thereof, wherein said at least one substrate molecule is a substrate of at least one catalytic macromolecule, and wherein said catalytic macromolecule catalyzes the formation of at least one electroactive product using said substrate/s; and
(e) performing an electrochemical voltammetry or amperometry analysis of said sample to detect the production of at least one electroactive product; wherein the detection of said product indicates the presence and/or activity of said catalytic macromolecule in said target pathogen lysates;
thereby diagnosing an infectious disease caused by at least one pathogen in the subject, and identifying and/or quantifying the pathogen and/or profiling the catalytic activity of said pathogen in the subject.

69. The diagnostic method according to claim 68, wherein at least one of:
(I) the step of detecting, identifying, quantifying and/or catalytically profiling one or more target pathogens in at least sample of said subject is performed by a method comprising: performing an EIS analysis of said sample, comprising:
(i) contacting said sample with at least one first working electrode, at least one first reference electrode, and at least one first counter electrode, said at least one first working electrode is connected directly or indirectly to at least one target binding site and/or moiety;
(ii) measuring electrical currents between said at least one first working electrode and said at least one first reference electrode in response to alternating electric voltages at different frequencies applied between said at least one first working electrode and said at least one first counter electrode;
(iii) determining electrical impedances based on the measured electrical current and the electric voltages applied at the different frequencies;
(iv) determining a charge transfer electrical resistance based on the determined impedances; and determining presence and/or quantity of said at least one target pathogen captured to said at least one target binding site and/or moiety in accordance with said charge transfer electrical resistance; and
(II) wherein said pathogen is at least one T3SS expressing pathogen, wherein profiling the catalytic activity of said pathogen comprises profiling antibiotic resistance of the T3SS pathogen in the subject, and wherein said method is for the diagnosis of an infectious condition caused by or associated with at least one T3SS expressing pathogen.

70. A method of treating, preventing, ameliorating, reducing or delaying the onset of an infection by at least one bacteria expressing at least one T3SS in a subject in need thereof, the method comprising:
(a) classifying a subject as infected by said bacteria if the presence of at least one T3SS component is determined in at least one sample of said subject;
(b) determining the antibiotic resistance profile of said bacteria in a sample of said subject;
wherein determination of the presence of said at least one T3SS component in said sample, and profiling the antibiotic resistance of said bacteria is performed by the steps of:
(i) contacting at least one sample of said subject with at least one first electrode arrangement comprising at least one first working electrode, or with any unit or system thereof, said at least one first working electrode is connected directly or indirectly to at least one target binding site and/or moiety for binding and/or capturing one or more target pathogens from said sample;
(ii) performing an EIS analysis of said sample; wherein impedance variations indicate the presence and/or quantity of said target pathogen in said sample;
(iii) applying disruption conditions to a target pathogen bound to, or captured by, the target binding site and/or moiety thereby generating target pathogen lysates;
(iv) contacting said target pathogen lysates with one or more substrate molecules connected directly or indirectly to one or more second working electrodes of at least one second electrode arrangement, or with any unit or system thereof, wherein said at least one substrate molecule is a substrate of at least one enzyme providing antibiotic resistance to said pathogen, and wherein said enzyme catalyzes the conversion of said substrate molecule to form at least one electroactive product;
(v) performing an electrochemical voltammetry or amperometry analysis of said sample to detect the production of at least one electroactive product; and
wherein the detection of said product indicates the presence and/or activity of said enzyme in said target pathogen lysates, thereby profiling the antibiotic resistance of said target pathogen in said sample; and
(c) administering to a subject classified as an infected subject in step (a), a therapeutically effective amount of at least one anti-bacterial agent, in accordance with the antibiotic resistance profile determined in step (b), wherein the determination of the presence of said at least one T3SS component in said sample is performed by the method as defined by claim 63.
